(19) United States
(12) Patent Application Publication
(10) Pub. No.: US 2024/0238297 A1
(43) Pub. Date: Jul. 18, 2024

(54) USES AND METHODS FOR RECURRENT PRIMARY CNS NEOPLASMS

(71) Applicant: CHIMERIX, INC., Durham, NC (US)

(72) Inventors: Joshua Edward Allen, Durham, NC (US); Jill Christine CHAPPELL, Durham, NC (US); Andrew Kang-Kang LEE, Durham, NC (US); Odin Johann NADERER, Chapel Hill, NC (US); Varun Vijay PRABHU, Philadelphia, PA (US); Phiroze Behram SETHNA, Durham, NC (US)

(21) Appl. No.: 18/559,992

(22) PCT Filed: May 13, 2022
(86) PCT No.: PCT/US2022/072304

§ 371 Date: Nov. 9, 2023

Related U.S. Application Data
(60) Provisional application No. 63/188,133, filed on May 13, 2021.

(51) Int. Cl. A61K 31/519; A61P 35/00

(52) U.S. Cl. CPC A61K 31/519; A61P 35/00

(57) ABSTRACT

This disclosure relates, at least in part, to a method of treatment. In one embodiment, the method of treatment comprises administering to a subject in need of such treatment at least a first therapeutic agent including ONC-206, 7-benzyl-4-(2,4-difluorobenzyl)-2,4,6,7,8,9-hexahydroimidazo[1,2-a]pyrido[3,4-e]pyrimidin-5(1H)-one, for the treatment of one or more CNS neoplasms.

### CROSS-REFERENCE TO RELATED APPLICATIONS

The present application is a PCT International Application claiming priority to U.S. Provisional Application No. 63/188,133 filed May 13, 2021, herein incorporated by reference in its entirety.

### BACKGROUND OF THE INVENTION

The majority of recurrent tumor types, such as CNS, neuroendocrine, and endometrial tumors, lack effective systemic therapy options following surgical resection and adjuvant radiotherapy. ONC201, which may be referred to as Compound 1 or NSC 350625, herein, is a founding member of the imipridone class of small molecules, and has induced durable tumor regressions in patients with diffuse midline glioma, H3 K27M-mutant (DMG H3K27M).

ONC206, which may be referred to as Compound 2 herein, is the second imipridone to enter clinical development, is a DRD2 antagonist and CIpP agonist that exhibits differentiated receptor pharmacology and gene expression profiles in tumors relative to ONC201.

The structures for each of compounds is as follows:

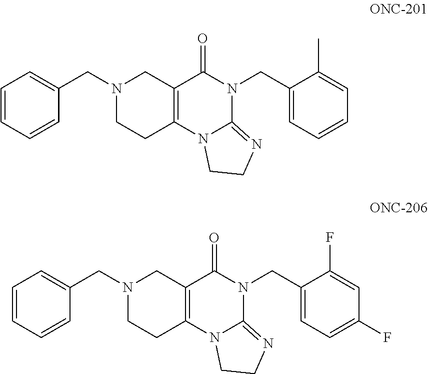 [SMILES: Cc1ccccc1CN1C(=O)C2=C(CCN(Cc3ccccc3)C2)N2CCN=C12 ; O=C1C2=C(CCN(Cc3ccccc3)C2)N2CCN=C2N1Cc1ccc(F)cc1F]

ONC-201 may also be referred to as 7-benzyl-4-(2-methylbenzyl)-2,4,6,7,8,9-hexahydroimidazo[1,2-a]pyrido[3,4-e]pyrimidin-5(1H)-one, and in one embodiment is provided as the dihydrochloride salt

ONC-206 may also be referred to as 7-benzyl-4-(2,4-difluorobenzyl)-2,4,6,7,8,9-hexahydroimidazo[1,2-a]pyrido[3,4-e]pyrimidin-5(1H)-one, and in one embodiment, is provided as the dihydrochloride salt.

### BRIEF SUMMARY OF THE INVENTION

One embodiment of the present disclosure includes a method of treating one or more cancer, including one or more CNS neoplasm, comprising administering ONC-206, 7-benzyl-4-(2,4-difluorobenzyl)-2,4,6,7,8,9-hexahydroimidazo[1,2-a]pyrido[3,4-e]pyrimidin-5(1H)-one, or a salt thereof.

In one aspect, the CNS neoplasm is one or more tumor represented in the 2021 WHO Classification of Tumors of the Central Nervous System, Neuro-Oncology, Volume 23, Issue 8, August 2021, Pages 1231 to 1251, https://doi.org/101093/neuonc/noab106, Published 29 Jun. 2021, and herein incorporated by reference with regard to such classification.

In one aspect, the CNS neoplasm is one or more of Gliomas, glioneuronal tumors, and neuronal tumors, Adult-type diffuse gliomas, Astrocytoma, IDH-mutant, Oligodendroglioma, IDH-mutant, and 1p/19q-codeleted, Glioblastoma, IDH-wildtype, Pediatric-type diffuse low-grade gliomas, Diffuse astrocytoma, MYB- or MYBL1-altered, Angiocentric glioma, Polymorphous low-grade neuroepithelial tumor of the young, Diffuse low-grade glioma, MAPK pathway-altered, Pediatric-type diffuse high-grade gliomas, Diffuse midline glioma, H3 K27-altered, Diffuse hemispheric glioma, H3 G34-mutant, Diffuse pediatric-type high-grade glioma, H3-wildtype and IDH-wildtype, Infant-type hemispheric glioma, Circumscribed astrocytic glioma, Pilocytic astrocytoma, High-grade astrocytoma with piloid features, Pleomorphic xanthoastrocytoma, Subependymal giant cell astrocytoma, Chordoid glioma, Astroblastoma, MN1-altered, Glioneuronal and neuronal tumors, Ganglioglioma, Desmoplastic infantile ganglioglioma/desmoplastic infantile astrocytoma, Dysembryoplastic neuroepithelial tumor, Diffuse glioneuronal tumor with oligodendroglioma-like features and nuclear clusters, Papillary glioneuronal tumor, Rosette-forming glioneuronal tumor, Myxoid glioneuronal tumor, Diffuse leptomeningeal glioneuronal tumor, Gangliocytoma, Multinodular and vacuolating neuronal tumor, Dysplastic cerebellar gangliocytoma (Lhermitte-Duclos disease), Central neurocytoma, Extraventricular neurocytoma, Cerebellar liponeurocytoma, Ependymal tumors, Supratentorial ependymoma, Supratentorial ependymoma, ZFTA fusion-positive, Supratentorial ependymoma, YAP1 fusion-positive, Posterior fossa ependymoma, Posterior fossa ependymoma, group PFA, Posterior fossa ependymoma, group PFB, Spinal ependymoma, Spinal ependymoma, MYCN-amplified, Myxopapillary ependymoma, Subependymoma, Choroid plexus tumors, Choroid plexus papilloma, Atypical choroid plexus papilloma, Choroid plexus carcinoma, Embryonal tumors, Medulloblastoma, Medulloblastomas, molecularly defined, Medulloblastoma, WNT-activated, Medulloblastoma, SHH-activated and TP53-wildtype, Medulloblastoma, SHH-activated and TP53-mutant, Medulloblastoma, non-WNT/non-SHH, Medulloblastomas, histologically defined, Other CNS embryonal tumors, Atypical teratoid/rhabdoid tumor, Cribriform neuroepithelial tumor, Embryonal tumor with multilayered rosettes, CNS neuroblastoma, FOXR2-activated, CNS tumor with BCOR internal tandem duplication, CNS embryonal tumor, Pineal tumors, Pineocytoma, Pineal parenchymal tumor of intermediate differentiation, Pineoblastoma, Papillary tumor of the pineal region, Desmoplastic myxoid tumor of the pineal region, SMARCB1-mutant, Cranial and paraspinal nerve tumors, Schwannoma, Neurofibroma, Perineurioma, Hybrid nerve sheath tumor, Malignant melanotic nerve sheath tumor, Malignant peripheral nerve sheath tumor, Paraganglioma, Meningiomas, Meningioma, Mesenchymal, non-meningothelial tumors, Soft tissue tumors, Fibroblastic and myofibroblastic tumors, Solitary fibrous tumor, Vascular tumors, Hemangiomas and vascular malformations, Hemangioblastoma, Skeletal muscle tumors, Rhabdomyosarcoma, Uncertain differentiation, Intracranial mesenchymal tumor, FET-CREB fusion-positive, CIC-rearranged sarcoma, Primary intracranial sarcoma, DICER1-mutant, Ewing sarcoma, Chondro-osseous tumors, Chondrogenic tumors, Mesenchymal chondrosarcoma, Chondrosarcoma, Notochordal tumors, Chordoma (including poorly differentiated chordoma), Melanocytic tumors, Diffuse meningeal melanocytic neoplasms, Meningeal melanocytosis and meningeal melanomatosis, Circumscribed meningeal melanocytic neoplasms, Meningeal melanocytoma and meningeal melanoma, Hematolymphoid tumors, Lymphomas, CNS lymphomas, Primary diffuse large B-cell lymphoma of the CNS, Immunodeficiency-associated CNS lymphoma, Lymphomatoid granulomatosis, Intravascular large B-cell lymphoma, Miscellaneous rare lymphomas in the CNS, MALT lymphoma of the dura, Other low-grade B-cell lymphomas of the CNS, Anaplastic large cell lymphoma (ALK+/ALK−), T-cell and NK/T-cell lymphomas, Histiocytic tumors, Erdheim-Chester disease, Rosai-Dorfman disease, Juvenile xanthogranuloma, Langerhans cell histiocytosis, Histiocytic sarcoma, Germ cell tumors, Mature teratoma, Immature teratoma, Teratoma with somatic-type malignancy, Germinoma, Embryonal carcinoma, Yolk sac tumor, Choriocarcinoma, Mixed germ cell tumor, Tumors of the sellar region, Adamantinomatous craniopharyngioma, Papillary craniopharyngioma, Pituicytoma, granular cell tumor of the sellar region, and spindle cell oncocytoma, Pituitary adenoma/PitNET, Pituitary blastoma, Metastases to the CNS, Metastases to the brain and spinal cord parenchyma, and Metastases to the meninges.

In one aspect, the CNS neoplasm is one or more of Pilocytic astrocytomas, Diffuse astrocytomas, Anaplastic astrocytomas, Glioblastomas, Oligodendroglial tumors, Ependymal tumor, Medulloblastomas, Pineal tumors, Meningeal tumors, and Germ cell tumors. In one aspect, the administration reduces one or more symptom of the CNS neoplasm. In one aspect, the administration reduces tumor growth. In one aspect, the administration provides one or more of (i) reduction in tumor size, (ii) progression-free survival, (iii) overall survival, (iv) patient-reported outcomes, (v) disease-free survival, (vi) objective response, (vii) complete response, (viii) increased time to progression, (ix) reduced corticosteroid use, (x) reduced supportive medication, (xi) reduced incidence of seizures, (xii) reduced use of anti seizure medication, (xiii) increased qualify of life, (xiv) reduced neurological deficits, and (xv) other objective response. In one aspect, the administration is made in combination with one or more additional therapy or therapeutic agent. In one aspect, the dose of ONC-206 or a salt thereof is from about 25 mg to about 75 mg, based on free base form. In one aspect, an upper dose may be about 2 to 3 grams. In one aspect, the dose is about 50 mg. In one aspect, the dose of ONC-206 or a salt thereof is from about 5 mg/kg to about 100 mg/kg. In one aspect, the dose is about 50 mg/kg. In one aspect, the dose of ONC-206 or a salt thereof at a volume of 10 ml/kg is from about 0.6 mg/l to about 10 mg/ml. In one aspect, the dose is about 5 mg/ml. In one aspect, the dose of ONC-206 or a salt thereof is from about 12.5 mg/kg/day to about 25 mg/kg/day. In one aspect, the dose is about 12.5 mg/kg/day. In one aspect, the dose of ONC-206 or a salt thereof is from about 8 mg/kg to about 20 mg/kg. In one aspect, the dose of ONC-206 or a salt thereof is from about 5 mg/kg/day to about 50 mg/kg/day. In one aspect, the dose is less than about 50 mg/kg/day. In one aspect, the dose of ONC-206 or a salt thereof is from about 1.7 mg/kg/day to about 16.7 mg/kg/day. In one aspect, the dose is greater than about 16.7 mg/kg/day. In one aspect, the dose provided is dose provided is daily, twice a week, three times a week, four times a week, five times a week, six times a week, weekly, bi-weekly, or monthly. In one aspect, the dose is three times a day, twice daily, daily, every other day, every third day, every fourth day, every fifth day, every sixth day, or weekly. In one aspect, the Cmax is from about 4 μM to about 20 μM. In one aspect, the terminal half-life is about 6 hours. In one aspect, a target tissue distribution relative to plasma ONC-206 concentration is at least one or more of 2 fold, 3 fold, 4 fold, 5 fold, 6 fold, 7 fold, 8 fold, 9 fold, or 10 fold higher. In one aspect, the ONC-206 is the di-HCl salt. In one aspect, the treatment relates to a recurrent neoplasm. In one aspect, the treatment is other than a first line treatment. In one aspect, the treatment is to an advanced cancer. In one aspect, the treatment is administered at least 30 days post-radiation. In one aspect, the treatment is administered at least 60 days post-radiation. In one aspect, the treatment is administered at least 90 days post-radiation. In one aspect, the treatment is administered after surgical resection. In one aspect, the CNS neoplasm is one or more of recurrent glioblastoma, WHO Grade 2 and 3 infiltrating glialneoplasms, DMG H3K27M, DMG H3 K27-altered, DMG H3 K27me-loss (H3K27me3), ependymoma, medulloblastoma, malignantmeningiomas, and other rare primary CNS neoplasms. In one aspect, the administration is monitored with one or more of DRD2, DRD2 dimer, CIpP, CIpP substrates such as SDHA, SDHB, and markers of oxidative phosphorylation, DRD5, c-myc, and n-myc expression. In one aspect, an objective response rate is measured by one or more of RANO criteria, RECIST criteria overall survival, progression-free survival, and disease control rate.

One embodiment of the present disclosure includes use of ONC-206, 7-benzyl-4-(2,4-difluorobenzyl)-2,4,6,7,8,9-hexahydroimidazo[1,2-a]pyrido[3,4-e]pyrimidin-5(1H)-one, or a salt thereof, in the preparation of a medicament for treating one or more CNS neoplasm.

In one aspect, the CNS neoplasm is one or more of Pilocytic astrocytomas, Diffuse astrocytomas, Anaplastic astrocytomas, Glioblastomas, Oligodendroglial tumors, Ependymal tumor, Medulloblastomas, Pineal tumors, Meningeal tumors, and Germ cell tumors. In one aspect, the administration reduces one or more symptom of the CNS neoplasm. In one aspect, the administration reduces tumor growth. In one aspect, the administration provides one or more of (i) reduction in tumor size, (ii) progression-free survival, (iii) overall survival, (iv) patient-reported outcomes, (v) disease-free survival, (vi) objective response, (vii) complete response, and (viii) increased time to progression. In one aspect, the administration is made in combination with one or more additional therapy or therapeutic agent. In one aspect, the dose of ONC-206 or a salt thereof is from about 25 mg to about 75 mg, based on free base form. In one aspect, wherein the dose is about 50 mg. In one aspect, the dose of ONC-206 or a salt thereof is from about 5 mg/kg to about 100 mg/kg. In one aspect, the dose is about 50 mg/kg. In one aspect, the dose of ONC-206 or a salt thereof at a volume of 10 ml/kg is from about 0.6 mg/l to about 10 mg/ml. In one aspect, the dose is about 5 mg/ml. In one aspect, the dose of ONC-206 or a salt thereof is from about 12.5 mg/kg/day to about 25 mg/kg/day. In one aspect, the dose is about 12.5 mg/kg/day. In one aspect, the dose of ONC-206 or a salt thereof is from about 8 mg/kg to about 20 mg/kg. In one aspect, the dose of ONC-206 or a salt thereof is from about 5 mg/kg/day to about 50 mg/kg/day. In one aspect, the dose is less than about 50 mg/kg/day. In one aspect, the dose of ONC-206 or a salt thereof is from about 1.7 mg/kg/day to about 16.7 mg/kg/day. In one aspect, the dose is greater than about 16.7 mg/kg/day. In one aspect, dose provided is daily, twice a week, three times a week, four times a week, five times a week, six times a week, weekly, bi-weekly, or monthly. In one aspect, the dose is three times a day, twice daily, daily, every other day, every third day, every fourth day, every fifth day, every sixth day, or weekly. In one aspect, the Cmax is from about 4 μM to about 20 μM. In one aspect, the terminal half-life is about 6 hours. In one aspect, a target tissue distribution relative to plasma ONC-206 concentration is at least one or more of 2 fold, 3 fold, 4 fold, 5 fold, 6 fold, 7 fold, 8 fold, 9 fold, or 10 fold higher. In one aspect, the ONC-206 is the di-HCl salt. In one aspect, the treatment relates to a recurrent neoplasm. In one aspect, the treatment is other than a first line treatment. In one aspect, the treatment is to an advanced cancer. In one aspect, the treatment is administered at least 30 days post-radiation. In one aspect, the treatment is administered at least 60 days post-radiation. In one aspect, the treatment is administered at least 90 days post-radiation. In one aspect, the treatment is administered after surgical resection. In one aspect, the CNS neoplasm is one or more of recurrent glioblastoma, WHO Grade 2 and 3 infiltrating glialneoplasms, DMG H3K27M, ependymoma, medulloblastoma, malignantmeningiomas, and other rare primary CNS neoplasms. In one aspect, the administration is monitored with one or more of DRD2, DRD2 dimer, CIpP, DRD5, c-myc, and n-myc expression. In one aspect, an objective response rate is measured by one or more of RANO criteria, overall survical, progression-free survival, and disease control rate.

One embodiment of the present disclosure includes a compound ONC-206, 7-benzyl-4-(2,4-difluorobenzyl)-2,4,6,7,8,9-hexahydroimidazo[1,2-a]pyrido[3,4-e]pyrimidin-5(1H)-one, or a salt thereof, for use in the preparation of a medicament for treating one or more CNS neoplasm.

In one aspect, the CNS neoplasm is one or more of Pilocytic astrocytomas, Diffuse astrocytomas, Anaplastic astrocytomas, Glioblastomas, Oligodendroglial tumors, Ependymal tumor, Medulloblastomas, Pineal tumors, Meningeal tumors, and Germ cell tumors. In one aspect, the administration reduces one or more symptom of the CNS neoplasm. In one aspect, the administration reduces tumor growth. In one aspect, the administration provides one or more of (i) reduction in tumor size, (ii) progression-free survival, (iii) overall survival, (iv) patient-reported outcomes, (v) disease-free survival, (vi) objective response, (vii) complete response, and (viii) increased time to progression. In one aspect, the administration is made in combination with one or more additional therapy or therapeutic agent. In one aspect, the dose of ONC-206 or a salt thereof is from about 25 mg to about 75 mg, based on free base form. In one aspect, wherein the dose is about 50 mg. In one aspect, the dose of ONC-206 or a salt thereof is from about 5 mg/kg to about 100 mg/kg. In one aspect, the dose is about 50 mg/kg. In one aspect, the dose of ONC-206 or a salt thereof at a volume of 10 ml/kg is from about 0.6 mg/l to about 10 mg/ml. In one aspect, the dose is about 5 mg/ml. In one aspect, the dose of ONC-206 or a salt thereof is from about 12.5 mg/kg/day to about 25 mg/kg/day. In one aspect, the dose is about 12.5 mg/kg/day. In one aspect, the dose of ONC-206 or a salt thereof is from about 8 mg/kg to about 20 mg/kg. In one aspect, the dose of ONC-206 or a salt thereof is from about 5 mg/kg/day to about 50 mg/kg/day. In one aspect, the dose is less than about 50 mg/kg/day. In one aspect, the dose of ONC-206 or a salt thereof is from about 1.7 mg/kg/day to about 16.7 mg/kg/day. In one aspect, the dose is greater than about 16.7 mg/kg/day. In one aspect, the dose provided is dose provided is daily, twice a week, three times a week, four times a week, five times a week, six times a week, weekly, bi-weekly, or monthly. In one aspect, the dose is three times a day, twice daily, daily, every other day, every third day, every fourth day, every fifth day, every sixth day, or weekly. In one aspect, the Cmax is from about 4 μM to about 20 μM. In one aspect, the terminal half-life is about 6 hours. In one aspect, a target tissue distribution relative to plasma ONC-206 concentration is at least one or more of 2 fold, 3 fold, 4 fold, 5 fold, 6 fold, 7 fold, 8 fold, 9 fold, or 10 fold higher. In one aspect, the ONC-206 is the di-HCl salt. In one aspect, the treatment relates to a recurrent neoplasm. In one aspect, the treatment is other than a first line treatment. In one aspect, the treatment is to an advanced cancer. In one aspect, the treatment is administered at least 30 days post-radiation. In one aspect, the treatment is administered at least 60 days post-radiation. In one aspect, the treatment is administered at least 90 days post-radiation. In one aspect, the treatment is administered after surgical resection. In one aspect, the CNS neoplasm is one or more of recurrent glioblastoma, WHO Grade 2 and 3 infiltrating glialneoplasms, DMG H3K27M, ependymoma, medulloblastoma, malignantmeningiomas, and other rare primary CNS neoplasms. In one aspect, the administration is monitored with one or more of DRD2, DRD2 dimer, CIpP, DRD5, c-myc, and n-myc expression. In one aspect, an objective response rate is measured by one or more of RANO criteria, overall survical, progression-free survival, and disease control rate.

One embodiment of the present disclosure includes treating one or more cancer in a subject in need thereof comprising administering ONC-206: 7-benzyl-4-(2,4-difluorobenzyl)-2,4,6,7,8,9-hexahydroimidazo[1,2-a]pyrido[3,4-e]pyrimidin-5(1H)-one, or a salt thereof, at a dose of about 50 mg twice daily for three consecutive days.

In one aspect, the administering for three consecutive days is followed by four days without dosing ONC-206, which may be referred to as a drug holiday. In one aspect, the cancer is a CNS neoplasm selected from the group consisting of one or more of Pilocytic astrocytomas, Diffuse astrocytomas, Anaplastic astrocytomas, Glioblastomas, Oligodendroglial tumors, Ependymal tumor, Medulloblastomas, Pineal tumors, Meningeal tumors, and Germ cell tumors. In one aspect, the administration reduces one or more symptom of the cancer. In one aspect, the administration reduces tumor growth. In one aspect, the administration provides one or more of (i) reduction in tumor size, (ii) progression-free survival, (iii) overall survival, (iv) patient-reported outcomes, (v) disease-free survival, (vi) objective response, (vii) complete response, and (viii) increased time to progression. In one aspect, total weekly dose of ONC-206 is about 300 mg. In one aspect, the total weekly AUClast is lower than about 5270 hr*ng/mL.

One embodiment of the present disclosure includes a dosing regimen comprising: administering a dose of ONC-206: 7-benzyl-4-(2,4-difluorobenzyl)-2,4,6,7,8,9-hexahydroimidazo[1,2-a]pyrido[3,4-e]pyrimidin-5(1H)-one, or a salt thereof, twice daily for at least one day followed by a drug holiday of at least one day.

In one aspect, ONC-206 is administered twice daily for two or more consecutive days followed by a drug holiday of at least one day. In one aspect, ONC-206 is administered twice daily for two or more consecutive days followed by a drug holiday of at least two consecutive days. In one aspect, ONC-206 is administered twice daily for three or more consecutive days followed by a drug holiday of at least two consecutive days. In one aspect, ONC-206 is administered twice daily for three or more consecutive days followed by a drug holiday of at least three consecutive days. In one aspect, ONC-206 is administered twice daily for three or more consecutive days followed by a drug holiday of at least four consecutive days. In one aspect, the dose of ONC-206 is from about 5 mg to about 150 mg. In one aspect, the dose of ONC-206 is from about 25 mg to about 100 mg. In one aspect, the dose of ONC-206 is one of 25 mg, 50 mg, 75 mg, or 100 mg. In one aspect, the dose of ONC-206 is 50 mg.

The preceding is a summary to provide an introduction and understanding of some embodiments of the present disclosure. This summary is neither an extensive nor exhaustive presentation of the present disclosure and its various embodiments. As will be appreciated, other embodiments of the present disclosure are possible utilizing, alone or in combination, one or more of the features, embodiments, and aspects set forth above or described in detail below. Each of those combinations of features, embodiments, and aspects should be considered as disclosed embodiments themselves.

### BRIEF DESCRIPTION OF THE DRAWINGS

This brief description, as well as the following detailed description of embodiments of the present disclosure, will be better understood when read in conjunction with the appended drawings of an exemplary embodiment. It should be understood, however, that the disclosure is not limited to the precise arrangements and instrumentalities shown.

FIG. 1 illustrates an exemplary pharmacokinetic profile of ONC206 in Sprague Dawley rats following a single oral gavage dose (PO) of 50 and 125 mg/kg. 10000 ng/ml represents ˜20 μM.

FIG. 2 illustrates graphical results of an exemplary rat biodistribution study of ONC206 with 50 mg/kg PO. Plasma and tissues concentrations depicted over time after ONC206 administration.

FIG. 3 illustrates a mechanism of action for ONC-206. As shown, ONC206 antagonizes DRD2 at the cell surface, resulting in activation of ISR involving ATF4/CHOP induction and upregulation of DR5 and TRAIL gene expression to induce apoptotic tumor cell death.

FIGS. 4 and 5 illustrate in vitro efficacy of ONC206 in human cancer cell lines.

FIG. 4 is a graphical illustration of In vitro sensitivity of >1000 Genomics of Drug Sensitivity in Cancer (GDSC) human cancer cell lines to ONC206 (72 h) averaged and organized by tumor type. The results are shown as completeness of ONC206 response quantified as the average area under the curve (AUC) in the dose-response cell viability curve among all cell lines in each tumor type. Error bars represent standard error of mean.

FIG. 5 illustrates average GI50 with 72 hour ONC206 (0.078-20 μM) treatment in a panel of Ewings sarcoma, neuroblastoma and medulloblastoma cell lines in the GDSC screen.

FIGS. 6A and 6B illustrate n vivo antitumor efficacy of ONC206 at 50 mg/kg once a week without body weight loss. (A) shows Tumor volume of HuCCT1 xenografts in athymic nude mice and (B) shows associated body weight following continuous treatment with ONC206 50 mg/kg PO and vehicle once a week (n=6). * p<0.05

FIG. 7 is a graphical illustration of data presented as mean plasma ONC206 concentration profiles detected by LC-MS-MS on Cycle 1 Day 1, except for 200 mg data which is presented as the profile of the single patient receiving the 200 mg dose. Error bars represent standard deviation. Nominal time is relative to administration of the first dose at 0 hr. Patients received ONC206 oral capsules.

FIG. 8 is a graphical illustration of H4 glioma cells were treated with ONC206 at indicated concentrations ranging from 5.1 nM to 11 μM. At indicated time points, cells were washed with PBS and media was replaced to washout ONC206. Cell viability was determined on day 7 post initial treatment by Celltiter-Glo. At 72 h, IC50=337 nM and IC90=715 nM. Error bars represent standard error of mean (n=4).

FIG. 9 is a graphical illustration of mean ONC206 plasma concentration projections following BID dosing regimens for three consecutive days in adult patients.

### DETAILED DESCRIPTION OF THE INVENTION

As noted hereinabove, the majority of recurrent CNS tumors lack effective systemic therapy options following surgical resection and adjuvant radiotherapy. ONC201, which may be referred to as Compound 1 or NSC 350625, herein, is the founding member of the imipridone class of small molecules, and has induced durable tumor regressions in patients with diffuse midline glioma, H3 K27M-mutant (DMG H3K27M). ONC206, which may be referred to as Compound 2 herein, is the second imipridone to enter clinical development, is a DRD2 antagonist and CIpP agonist that exhibits differentiated receptor pharmacology and gene expression profiles in tumors relative to ONC201.

### I. PHARMACEUTICAL COMPOSITIONS

One embodiment of the present disclosure includes a pharmaceutical composition, comprising ONC-206.

In one embodiment, the pharmaceutical composition comprises ONC-206 or a pharmaceutically acceptable mono-salt thereof. In one embodiment, the pharmaceutical composition comprises ONC-206 or a pharmaceutically acceptable di-salt thereof. In one embodiment, the pharmaceutical composition comprises ONC-206 or a pharmaceutically acceptable mono- or multi-salt (e.g., di-salt or tri-salt, where it is understood that throughout this disclosure a di-salt encompasses a tri-salt or other multi-salt) thereof selected from the group consisting of hydrochloride, hydrobromide, hydrogensulphate, sulfates, phosphates, fumarates, succinates, oxalates and lactates, bisulfates, hydroxyl, tartrate, nitrate, citrate, bitartrate, carbonate, malate, maleate, fumarate sulfonate, methylsulfonate, formate, and carboxylate. In one embodiment, the pharmaceutical composition comprises ONC-206 or a pharmaceutically acceptable salt thereof selected from the group consisting of p-toluene-sulfonate, benzenesulfonate, methanesulfonate, oxalate, succinate, tartrate, citrate, fumarate and maleate. In one embodiment, the pharmaceutical composition comprises ONC-206 or a pharmaceutically acceptable salt selected from the group consisting of ammonium, sodium, potassium, calcium, magnesium, zinc, lithium, and/or with counter-ions such as methylamino, dimethylamino, diethylamino and triethylamino counter-ions. In one embodiment, the pharmaceutical composition comprises ONC-206, a hydrochloride di-salt thereof (e.g., di-hydrochloride salt) or a hydrobromide di-salt thereof (e.g., di-hydrobromide salt).

In one embodiment, a pharmaceutical composition in accordance with the present disclosure includes a di-salt (e.g., a di-hydrochloride salt) of ONC-206. Salts (e.g., di-salts, tri-salts, or mutli-salts) of ONC-206 can be prepared from ONC-206, which can be obtained commercially or synthesized using standard chemical synthetic methodology known to one of ordinary skill in the art.

Dihydrochloride salts of compounds within the class of impiridones of which ONC-206 is a member, achieve unexpected technical effects. As one example, a comparison between the a dihydrochloride salt of ONC-201 and the corresponding free base demonstrates that the solubility of the dihydrochloride salt in water is greater than 50 mg/mL, while it is less than 1 mg/mL for the free base. In addition, after two months at 25° C. and at 40° C., the percentage of impurities in the dihydrochloride salt is not detected and 3%, respectively; while for the free base the corresponding the percentage of impurities is 20% and 24%, respectively. This technical effect appears to extend to the class of impiridones.

In one embodiment, the pharmaceutical composition in accordance with the present disclosure includes at least one pharmaceutically acceptable carrier. Suitable pharmaceutically acceptable carriers, included, but are not limited to, those found in Handbook of Pharmaceutical Excipients, 7th Edition, edited by Raymond C. Rowe et al., American Pharmaceutical Association, Washington, USA and Pharmaceutical Press, London; and earlier editions.

Exemplary pharmaceutically acceptable carriers, methods for making pharmaceutical compositions and various dosage forms, as well as modes of administration are well-known in the art, for example as detailed in Pharmaceutical Dosage Forms: Tablets, edited by Larry L. Augsburger and Stephen W. Hoag., London: Informa Healthcare, 2008; and in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

The pharmaceutical compositions of the present disclosure may be administered to a subject via any suitable route of administration. In one embodiment, the pharmaceutical composition is administered to a subject orally, parenterally, transdermally or transmucosally. In one embodiment, the pharmaceutical composition is administered to a subject in a parenteral dosage form. In one embodiment, the pharmaceutical composition is administered to a subject as a parenterally. In one embodiment, the pharmaceutical composition is administered to a subject via a parenteral route of administration selected from one or more of the group consisting of intravenous (IV), subcutaneous (SC), intramuscular (IM), and intrathecal. In one embodiment, the pharmaceutical composition is administered to a subject via a route of administration selected from rectal (PR) and transdermal. In one embodiment, the pharmaceutical composition is administered to a subject in a dosage form selected from the group consisting of sterile solutions, suspensions, suppositories, tablets and capsules. In one embodiment, the pharmaceutical composition is administered to a subject in an oral dosage form selected from the group consisting of a tablet, caplet, capsule, lozenge, syrup, liquid, suspension and elixir. In one embodiment, the pharmaceutical composition is administered to a subject in an oral dosage form selected from the group consisting of tablets, hard shell capsules, soft gelatin capsules, beads, granules, aggregates, powders, gels, solids and semi-solids.

In some embodiments, the pharmaceutical composition is in administered to a subject as a dosage form selected from the group consisting of sustained release forms, controlled release forms, delayed release forms and response release forms.

In some embodiments, the pharmaceutical composition of the present disclosure is formulated for ocular administration. In some embodiments, pharmaceutical compositions of the present disclosure are formulated for topical ocular administration. In some embodiments, the pharmaceutical compositions are formulated as ointments, drops, or liquids. In some embodiments, the pharmaceutical composition of the present disclosure can include conventional pharmaceutical carriers such as aqueous, powdery or oily bases, thickeners or the like.

In some embodiments, the pharmaceutical composition of the present disclosure is formulated as intravenous formulation. In one embodiment, the intravenous formulation comprises ONC-206 or a pharmaceutically acceptable salt of ONC-206 dissolved in a solvent. In one embodiment, the solvent comprises water. In one such embodiment, the intravenous formulation comprises ONC-206 or a pharmaceutically acceptable salt of ONC-206 dissolved in water at a concentration of 25 mg/ml. In some embodiments, the intravenous formulation includes a higher or a lower concentration of ONC-206 or a pharmaceutically acceptable salt thereof. In one embodiment, the intravenous formulation includes ONC-206 or a pharmaceutically acceptable salt thereof in a concentration of from about 5 mg/ml to about 100 mg/ml. In one embodiment, the intravenous formulation includes ONC-206 or a pharmaceutically acceptable salt thereof in a concentration of about 50 mg/ml. In one embodiment, the intravenous formulation includes ONC-206 or a pharmaceutically acceptable salt thereof in a concentration of about 5 mg/ml. In one embodiment, the intravenous formulation includes from about 0.5% to about 10% of ONC-206 or a pharmaceutically acceptable salt thereof. In one embodiment, the intravenous formulation includes from about 5% of ONC-206 or a pharmaceutically acceptable salt thereof.

In some embodiments, the intravenous formulation has pH of about 3. In one embodiment, pH of the intravenous formulation is adjusted to pH 3 with a phosphate buffer. In some embodiments, the intravenous formulation includes dextrose or sodium chloride. In one embodiment, the intravenous formulation including ONC-206 or or a pharmaceutically acceptable salt thereof in a concentration of about 5 mg/ml and pH 3 forms a stable solution. In one embodiment, the intravenous formulation includes ONC-206 or a pharmaceutically acceptable salt thereof in a concentration of about 5 mg/ml and pH<5 and forms a stable solution. In one embodiment, the intravenous formulation includes ONC-206 or a pharmaceutically acceptable salt thereof and one or more antioxidants. In one embodiment, the intravenous formulation includes a mixture of mono- and di-hydrochloride salt of ONC-206. In one embodiment, the intravenous formulation includes ONC-206 or a pharmaceutically acceptable salt thereof as a 1% solution having ONC-206 or tor a pharmaceutically acceptable salt thereof in a concentration of about 10 mg/ml. In one such embodiment, the intravenous formulation is a solution having a pH of about 3.3. In one embodiment, the pH is less than 4.0.

In one embodiment, a pharmaceutical composition according to the disclosure comprises about 0.1-99% of a salt of ONC-206 or a pharmaceutically acceptable salt thereof. In one such embodiment, the pharmaceutical composition further includes a pharmaceutically acceptable carrier. In one embodiment, a suitable pharmaceutically acceptable carrier includes an oil. In one embodiment, a suitable pharmaceutically acceptable carrier includes a sterile water. In one embodiment, a suitable pharmaceutically acceptable carrier includes an aqueous carrier.

In some embodiments, the intravenous formulation includes dextrose and/or sodium.

In one embodiment, the intravenous formulation comprises ONC-206 or a di-hydrochloride salt of ONC-206 dissolved in water at 25 mg/ml. In one such embodiment, the intravenous formulation is adjusted to pH 3 with phosphate buffer. In one such embodiment, the intravenous formulation includes dextrose or sodium chloride. In one such embodiment, the intravenous formulation includes a higher or a lower increase or decrease the concentration of the di-hydrochloride salt of ONC-206. In one embodiment, the intravenous formulation includes ONC-206 or a di-hydrochloride salt of ONC-206 in a concentration of about 5 mg/ml. In one embodiment, the intravenous formulation including ONC-206 or a di-hydrochloride salt of ONC-206 in a concentration of about 5 mg/ml and pH 3 forms a stable solution. In one embodiment, the intravenous formulation includes ONC-206 or a di-hydrochloride salt of ONC-206 in a concentration of about 5 mg/ml and pH<5 and forms a stable solution. In one embodiment, the intravenous formulation includes ONC-206 or a di-hydrochloride salt of ONC-206 and one or more antioxidants. In one embodiment, the intravenous formulation includes a mixture of mono- and di-hydrochloride salt of ONC-206. In one embodiment, the intravenous formulation includes ONC-206 or a di-hydrochloride salt of ONC-206 as a 1% solution having ONC-206 or the di-hydrochloride salt of ONC-206 in a concentration of about 10 mg/ml. In one such embodiment, the intravenous formulation is a solution having a pH of about 3.33. In one embodiment, the pH is less than 4.0.

In one embodiment, the intravenous formulation includes from about 0.5% to about 10% (or from about 5 mg/ml to about 100 mg/ml) of ONC-206 or a di-salt of ONC-206. In one embodiment, the intravenous formulation includes from about 5% (or about 50 mg/ml) of ONC-206 or a di-salt of ONC-206. In one embodiment, the intravenous infusion rate may be slowed to decrease side effects of ONC-206 or a di-salt of ONC-206.

In one embodiment, a pharmaceutical composition according to the disclosure comprises about 0.1-99% of a salt of ONC-206; and a pharmaceutically acceptable carrier, e.g., an oil or a sterile water or other aqueous carriers. In one embodiment, a pharmaceutical composition according to the disclosure comprises a mono or di-salt of ONC-206 in a range of from about 5% to about 50% for oral dosage forms.

In some embodiments, a pharmaceutical composition of the present disclosure includes an antioxidant. Suitable antioxidants include: ascorbic acid derivatives such as ascorbic acid, erythorbic acid, sodium ascorbate, thiol derivatives such as thioglycerol, cysteine, acetylcysteine, cystine, dithioerythreitol, dithiothreitol, glutathione, tocopherols, butylated hydroxyanisole (BHA), butylated hydroxytoluene (BHT), sulfurous acid salts such as sodium sulfate, sodium bisulfite, acetone sodium bisulfite, sodium metabisulfite, sodium sulfite, sodium formaldehyde sulfoxylate, and sodium thiosulfate, nordihydroguaiaretic acid. It should be noted that antioxidants used for aqueous formulations typically include: sodium sulphite, sodium metabisulphite, sodium formaldehyde sulphoxylate and ascorbic acid and combinations thereof, whereas antioxidants used in oil-based solutions, organic solvents, include butylated hydroxytoluene (BHT), butylated hydroxyanisole (BHA) and propyl gallate and combinations thereof. In yet other embodiments, an antioxidant can be one or more of a flavanoid, an isoflavone, monothioglycerol, L-cysteine, thioglycolic acid, α-tocopherol, ascorbic acid 6-palmitate, dihydrolipoic acid, butylated hydroxytoluene (BHT), butylated hydroxyanisole (BHA), vitamin E, propyl gallate, β-carotene, ascorbic acid. Antioxidants can typically be used in about 0.1% to 1.0% by weight, more typically about 0.2%.

In one embodiment, the pharmaceutical composition includes ONC-206 or a pharmaceutically acceptable salt thereof and at least one other therapeutic agent.

As used throughout the present disclosure, an additional, other, or second therapeutic agent may include a therapy as well, such as radiation or surgery, such as curative, preventative, diagnostic, staging, debulking, palliative, supporting, or restorative surgical procedures.

In one such embodiment, the at least one other therapeutic agent is selected from the group consisting of hormone analogues and antihormones, aromatase inhibitors, LHRH agonists and antagonists, inhibitors of growth factors, growth factor antibodies, growth factor receptor antibodies, tyrosine kinase inhibitors; antimetabolites; antitumour antibiotics; platinum derivatives; alkylation agents; antimitotic agents; tubuline inhibitors; PARP inhibitors, topoisomerase inhibitors, serine/threonine kinase inhibitors, tyrosine kinase inhibitors, protein protein interaction inhibitors, RAF inhibitors, MEK inhibitors, ERK inhibitors, IGF-1R inhibitors, ErbB receptor inhibitors, rapamycin analogs, BTK inhibitors, CRM1 inhibitors (e.g., KPT185), P53 modulators (e.g., Nutlins), antiangiogenics (e.g., axitinib, aflibercept, sorafenib, and regorafenib), amifostin, anagrelid, clodronat, filgrastin, interferon, interferon alpha, leucovorin, rituximab, procarbazine, levamisole, mesna, mitotane, pamidronate and porfimer, 2-chlorodesoxyadenosine, 2-fluorodesoxy-cytidine, 2-methoxyoestradiol, 2C4,3-alethine, 131-1-TM-601, 3CPA, 7-ethyl-10-hydroxycamptothecin, 16-aza-epothilone B, A 105972, A 204197, abiraterone, aldesleukin, alitretinoin, allovectin-7, altretamine, alvocidib, amonafide, anthrapyrazole, AG-2037, AP-5280, apaziquone, apomine, aranose, arglabin, arzoxifene, atamestane, atrasentan, auristatin PE, AVLB, AZ10992, ABX-EGF, AMG-479 (ganitumab), ARRY 162, ARRY 438162, ARRY-300, ARRY-142886/AZD-6244 (selumetinib), ARRY-704/AZD-8330, AR-12, AR-42, AS-703988, AXL-1717, AZD-8055, AZD-5363, AZD-6244, ARQ-736, ARQ 680, AS-703026 (primasertib), avastin, AZD-2014, azacytidine, azaepothilone B, azonafide, BAY-43-9006, BAY 80-6946, BBR-3464, BBR-3576, bevacizumab, BEZ-235, biricodar dicitrate, BCX-1777, BKM-120, bleocin, BLP-25, BMS-184476, BMS-247550, BMS-188797, BMS-275291, BMS-663513, BMS-754807, BNP-1350, BNP-7787, BIBW 2992 (afatinib, tomtovok), BIBF 1120 (vargatef), BI 836845, BI 2536, BI 6727, BI 836845, BI 847325, BI 853520, BUB-022, bleomycinic acid, bleomycin A, bleomycin B, brivanib, bryostatin-1, bortezomib, brostallicin, busulphan, BYL-719, CA-4 prodrug, CA-4, CapCell, calcitriol, canertinib, canfosfamide, capecitabine, carboxyphthalatoplatin, CCI-779, CC-115, CC-223, CEP-701, CEP-751, CBT-1 cefixime, ceflatonin, ceftriaxone, celecoxib, celmoleukin, cemadotin, CH4987655/RO-4987655, chlorotrianisene, cilengitide, ciclosporin, CDA-II, CDC-394, CKD-602, CKI-27, clofarabin, colchicin, combretastatin A4, COT inhibitors, CHS-828, CH-5132799, CLL-Thera, CMT-3 cryptophycin 52, CTP-37, CTLA-4 monoclonal antibodies, CP-461, CV-247, cyanomorpholinodoxorubicin, cytarabine, D 24851, decitabine, deoxorubicin, deoxyrubicin, deoxycoformycin, depsipeptide, desoxyepothilone B, dexamethasone, dexrazoxanet, diethylstilbestrol, diflomotecan, didox, DMDC, dolastatin 10, doranidazole, DS-7423, E7010, E-6201, edatrexat, edotreotide, efaproxiral, eflornithine, EGFR inhibitors, EKB-569, EKB-509, enzastaurin, enzalutamide, elsamitrucin, epothilone B, epratuzumab, ER-86526, erlotinib, ET-18-OCH3, ethynylcytidine, ethynyloestradiol, exatecan, exatecan mesylate, exemestane, exisulind, fenretinide, figitumumab, floxuridine, folic acid, FOLFOX, FOLFOX4, FOLFIRI, formestane, fotemustine, galarubicin, gallium maltolate, gefinitib, gemtuzumab, gimatecan, glufosfamide, GCS-100, GDC-0623, GDC-0941 (pictrelisib), GDC-0980, GDC-0032, GDC-0068, GDC-0349, GDC-0879, G17DT immunogen, GMK, GPX-100, gp100-peptide vaccines, GSK-5126766, GSK-690693, GSK-1120212 (trametinib), GSK-2118436 (dabrafenib), GSK-2126458, GSK-2132231A, GSK-2334470, GSK-2110183, GSK-2141795, GW2016, granisetron, herceptine, hexamethylmelamine, histamine, homoharringtonine, hyaluronic acid, hydroxyurea, hydroxyprogesterone caproate, ibandronate, ibrutinib, ibritumomab, idatrexate, idenestrol, IDN-5109, IGF-1R inhibitors, IMC-1C11, IMC-A12 (cixutumumab), immunol, indisulam, interferon alpha-2a, interferon alpha-2b, pegylated interferon alpha-2b, interleukin-2, INK-1117, INK-128, INSM-18, ionafarnib, ipilimumab, iproplatin, irofulven, isohomohalichondrin-B, isoflavone, isotretinoin, ixabepilone, JRX-2, JSF-154, J-107088, conjugated oestrogens, kahalid F, ketoconazole, KW-2170, KW-2450, lobaplatin, leflunomide, lenograstim, leuprolide, leuporelin, lexidronam, LGD-1550, linezolid, lutetium texaphyrin, lometrexol, losoxantrone, LU 223651, lurtotecan, LY-S6AKT1, LY-2780301, mafosfamide, marimastat, mechloroethamine, MEK inhibitors, MEK-162, methyltestosteron, methylprednisolone, MEDI-573, MEN-10755, MDX-H210, MDX-447, MDX-1379, MGV, midostaurin, minodronic acid, mitomycin, mivobulin, MK-2206, MK-0646 (dalotuzumab), MLN518, motexaf in gadolinium, MS-209, MS-275, MX6, neridronate, neratinib, Nexavar, neovastat, nilotinib, nimesulide, nitroglycerin, nolatrexed, norelin, N-acetylcysteine, 06-benzylguanine, oblimersen, omeprazole, oncophage, oncoVEXGM-CSF, ormiplatin, ortataxel, OX44 antibodies, OSI-027, OSI-906 (linsitinib), 4-1BB antibodies, oxantrazole, oestrogen, panitumumab, patupilone, PI3Ki, inhibitors, paxilisib, pegfilgrastim, PCK-3145, pegfilgrastim, PBI-1402, PBI-05204, PD0325901, PD-1 antibodies, PEG-paclitaxel, albumin-stabilized paclitaxel, PEP-005, PF-05197281, PF-05212384, PF-04691502, PHT-427, P-04, PKC412, P54, PI-88, pelitinib, pemetrexed, pentrix, perifosine, perillylalcohol, pertuzumab, PI3K inhibitors, PI3K/mTOR inhibitors, PG-TXL, PG2, PLX-4032/RO-5185426 (vemurafenib), PLX-3603/RO-5212054, PT-100, PWT-33597, PX-866, picoplatin, pivaloyloxymethylbutyrate, pixantrone, phenoxodiol O, PKI166, plevitrexed, plicamycin, polyprenic acid, porfiromycin, prednisone, prednisolone, quinamed, quinupristin, R115777, RAF-265, ramosetron, ranpirnase, RDEA-119/BAY 869766, RDEA-436, rebeccamycin analogues, receptor tyrosine kinase (RTK) inhibitors, regorafenib, revimid, RG-7167, RG-7304, RG-7421, RG-7321, RG 7440, rhizoxin, rhu-MAb, rinfabate, risedronate, rituximab, robatumumab, rofecoxib, RO-31-7453, RO-5126766, RO-5068760, RPR 109881A, rubidazone, rubitecan, R-flurbiprofen, RX-0201, S-9788, sabarubicin, SAHA, sargramostim, satraplatin, SB 408075, Se-015/Ve-015, SU5416, SU6668, SDX-101, semustin, seocalcitol, SM-11355, SN-38, SN-4071, SR-27897, SR-31747, SR-13668, SRL-172, sorafenib, spiroplatin, squalamine, suberanilohydroxamic acid, sutent, T 900607, T 138067, TAK-733, TAS-103, tacedinaline, talaporf in, Tarceva, tariquitar, tasisulam, taxotere, taxoprexin, tazarotene, tegafur, temozolamide, tesmilifene, testosterone, testosterone propionate, tesmilifene, tetraplatin, tetrodotoxin, tezacitabine, thalidomide, theralux, therarubicin, thymalfasin, thymectacin, tiazofurin, tipifarnib, tirapazamine, tocladesine, tomudex, toremofin, trabectedin, TransMID-107, transretinic acid, traszutumab, tremelimumab, tretinoin, triacetyluridine, triapine, triciribine, trimetrexate, TLK-286TXD 258, tykerb/tyverb, urocidin, valrubicin, vatalanib, vincristine, vinflunine, virulizin, WX-UK1, WX-554, vectibix, xeloda, XELOX, XL-147, XL-228, XL-281, XL-518/R-7420/GDC-0973, XL-765, YM-511, YM-598, ZD-4190, ZD-6474, ZD-4054, ZD-0473, ZD-6126, ZD-9331, ZD1839, ZSTK-474, zoledronat, zosuquidar, and combinations thereof.

In one embodiment, the at least one other therapeutic agent comprises one or more hormone analogues and/or antihormones are selected from the group consisting of tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxy-progesterone, octreotide, and combinations thereof. In one embodiment, the at least one other therapeutic agent comprises one or more LHRH agonists and/or antagonists selected from the group consisting of goserelin acetate, luprolide acetate, triptorelin pamoate and combinations thereof and wherein the LHRH antagonists are selected from the group consisting of Degarelix, Cetrorelix, Abarelix, Ozarelix, Degarelix combinations thereof. In one embodiment, the at least one other therapeutic agent comprises one or more growth factor inhibitors selected from the group consisting of inhibitors of: platelet derived growth factor (PDGF), fibroblast growth factor (FGF), vascular endothelial growth factor (VEGF), epidermal growth factor (EGF), insuline-like growth factors (IGF), human epidermal growth factor (HER) and hepatocyte growth factor (HGF). In one embodiment, the at least one other therapeutic agent comprises one or more inhibitors of the human epidermal growth factor selected from the group consisting of HER2, HER3, and HER4. In one embodiment, the at least one other therapeutic agent comprises one or more tyrosine kinase inhibitors selected from the group consisting of cetuximab, gefitinib, imatinib, lapatinib and trastuzumab, and combinations thereof. In one embodiment, the at least one other therapeutic agent comprises one or more aromatase inhibitors selected from the group consisting of anastrozole, letrozole, liarozole, vorozole, exemestane, atamestane, and combinations thereof. In one embodiment, the at least one other therapeutic agent comprises one or more antimetabolites which are antifolates selected from the group consisting of methotrexate, raltitrexed, and pyrimidine analogues. In one embodiment, the at least one other therapeutic agent comprises one or more antimetabolites which are pyrimidine analogues selected from the group consisting of 5-fluorouracil, capecitabin and gemcitabin. In one embodiment, the at least one other therapeutic agent comprises one or more antimetabolites which are purine and/or adenosine analogues selected from the group consisting of mercaptopurine, thioguanine, cladribine and pentostatin, cytarabine, fludarabine, and combinations thereof. In one embodiment, the at least one other therapeutic agent comprises one or more antitumour antibiotics selected from the group consisting of anthracyclins, doxorubicin, daunorubicin, epirubicin and idarubicin, mitomycin-C, bleomycin, dactinomycin, plicamycin, streptozocin and combinations thereof. In one embodiment, the at least one other therapeutic agent comprises one or more platinum derivatives selected from the group consisting of cisplatin, oxaliplatin, carboplatin and combinations thereof. In one embodiment, the at least one other therapeutic agent comprises one or more alkylation agents selected from the group consisting of estramustin, meclorethamine, melphalan, chlorambucil, busulphan, dacarbazin, cyclophosphamide, ifosfamide, temozolomide, nitrosoureas, and combinations thereof. In one embodiment, the at least one other therapeutic agent comprises nitrosoureas selected from the group consisting of carmustin, lomustin, thiotepa, and combinations thereof. In one embodiment, the at least one other therapeutic agent comprises antimitotic agents selected from the group consisting of Vinca alkaloids and taxanes. In one embodiment, the at least one other therapeutic agent comprises one or more taxanes selected from the group consisting of paclitaxel, docetaxel, and combinations thereof. In one embodiment, the at least one other therapeutic agent comprises one or more Vinca alkaloids selected from the group consisting of vinblastine, vindesin, vinorelbin, vincristine, and combinations thereof. In one embodiment, the at least one other therapeutic agent comprises one or more topoisomerase inhibitors which are epipodophyllotoxins. In one embodiment, the at least one other therapeutic agent comprises one or more epipodophyllotoxins selected from the group consisting of etoposide and etopophos, teniposide, amsacrin, topotecan, irinotecan, mitoxantron, and combinations thereof. In one embodiment, the at least one other therapeutic agent comprises one or more serine/threonine kinase inhibitors selected from the group consisting of PDK 1 inhibitors, B-Raf inhibitors, mTOR inhibitors, mTORC1 inhibitors, PI3K inhibitors, dual mTOR/PI3K inhibitors, STK 33 inhibitors, AKT inhibitors, PLK 1 inhibitors, inhibitors of CDKs, Aurora kinase inhibitors, and combinations thereof. In one embodiment, the at least one other therapeutic agent comprises one or more tyrosine kinase inhibitors which are PTK2/FAK inhibitors. In one embodiment, the at least one other therapeutic agent comprises one or more protein protein interaction inhibitors selected from the group consisting of IAP, Mcl-1, MDM2/MDMX and combinations thereof. In one embodiment, the at least one other therapeutic agent comprises one or more rapamycin analogs selected from the group consisting of everolimus, temsirolimus, ridaforolimus, sirolimus, and combinations thereof. In one embodiment, the at least one other therapeutic agent comprises one or more therapeutic agents selected from the group consisting of amifostin, anagrelid, clodronat, filgrastin, interferon, interferon alpha, leucovorin, rituximab, procarbazine, levamisole, mesna, mitotane, pamidronate and porfimer, and combinations thereof. In one embodiment, the at least one other therapeutic agent comprises one or more therapeutic agents selected from the group consisting of 2-chlorodesoxyadenosine, 2-fluorodesoxy-cytidine, 2-methoxyoestradiol, 2C4,3-alethine, 131-1-TM-601, 3CPA, 7-ethyl-10-hydroxycamptothecin, 16-aza-epothilone B, A 105972, A 204197, abiraterone, aldesleukin, alitretinoin, allovectin-7, altretamine, alvocidib, amonafide, anthrapyrazole, AG-2037, AP-5280, apaziquone, apomine, aranose, arglabin, arzoxifene, atamestane, atrasentan, auristatin PE, AVLB, AZ10992, ABX-EGF, AMG-479 (ganitumab), ARRY 162, ARRY 438162, ARRY-300, ARRY-142886/AZD-6244 (selumetinib), ARRY-704/AZD-8330, AR-12, AR-42, AS-703988, AXL-1717, AZD-8055, AZD-5363, AZD-6244, ARQ-736, ARQ 680, AS-703026 (primasertib), avastin, AZD-2014, azacytidine, azaepothilone B, azonafide, BAY-43-9006, BAY 80-6946, BBR-3464, BBR-3576, bevacizumab, BEZ-235, biricodar dicitrate, BCX-1777, BKM-120, bleocin, BLP-25, BMS-184476, BMS-247550, BMS-188797, BMS-275291, BMS-663513, BMS-754807, BNP-1350, BNP-7787, BIBW 2992 (afatinib, tomtovok), BIBF 1120 (vargatef), BI 836845, BI 2536, BI 6727, BI 836845, BI 847325, BI 853520, BUB-022, bleomycinic acid, bleomycin A, bleomycin B, brivanib, bryostatin-1, bortezomib, brostallicin, busulphan, BYL-719, CA-4 prodrug, CA-4, CapCell, calcitriol, canertinib, canfosfamide, capecitabine, carboxyphthalatoplatin, CCI-779, CC-115, CC-223, CEP-701, CEP-751, CBT-1 cefixime, ceflatonin, ceftriaxone, celecoxib, celmoleukin, cemadotin, CH4987655/RO-4987655, chlorotrianisene, cilengitide, ciclosporin, CDA-II, CDC-394, CKD-602, CKI-27, clofarabin, colchicin, combretastatin A4, COT inhibitors, CHS-828, CH-5132799, CLL-Thera, CMT-3 cryptophycin 52, CTP-37, CTLA-4 monoclonal antibodies, CP-461, CV-247, cyanomorpholinodoxorubicin, cytarabine, D 24851, decitabine, deoxorubicin, deoxyrubicin, deoxycoformycin, depsipeptide, desoxyepothilone B, dexamethasone, dexrazoxanet, diethylstilbestrol, diflomotecan, didox, DMDC, dolastatin 10, doranidazole, DS-7423, E7010, E-6201, edatrexat, edotreotide, efaproxiral, eflornithine, EGFR inhibitors, EKB-569, EKB-509, enzastaurin, enzalutamide, elsamitrucin, epothilone B, epratuzumab, ER-86526, erlotinib, ET-18-OCH3, ethynylcytidine, ethynyloestradiol, exatecan, exatecan mesylate, exemestane, exisulind, fenretinide, figitumumab, floxuridine, folic acid, FOLFOX, FOLFOX4, FOLFIRI, formestane, fotemustine, galarubicin, gallium maltolate, gefinitib, gemtuzumab, gimatecan, glufosfamide, GCS-100, GDC-0623, GDC-0941 (pictrelisib), GDC-0980, GDC-0032, GDC-0068, GDC-0349, GDC-0879, G17DT immunogen, GMK, GPX-100, gp100-peptide vaccines, GSK-5126766, GSK-690693, GSK-1120212 (trametinib), GSK-2118436 (dabrafenib), GSK-2126458, GSK-2132231A, GSK-2334470, GSK-2110183, GSK-2141795, GW2016, granisetron, herceptine, hexamethylmelamine, histamine, homoharringtonine, hyaluronic acid, hydroxyurea, hydroxyprogesterone caproate, ibandronate, ibrutinib, ibritumomab, idatrexate, idenestrol, IDN-5109, IGF-1R inhibitors, IMC-1C11, IMC-A12 (cixutumumab), immunol, indisulam, interferon alpha-2a, interferon alpha-2b, pegylated interferon alpha-2b, interleukin-2, INK-1117, INK-128, INSM-18, ionafarnib, ipilimumab, iproplatin, irofulven, isohomohalichondrin-B, isoflavone, isotretinoin, ixabepilone, JRX-2, JSF-154, J-107088, conjugated oestrogens, kahalid F, ketoconazole, KW-2170, KW-2450, lobaplatin, leflunomide, lenograstim, leuprolide, leuporelin, lexidronam, LGD-1550, linezolid, lutetium texaphyrin, lometrexol, losoxantrone, LU 223651, lurtotecan, LY-S6AKT1, LY-2780301, mafosfamide, marimastat, mechloroethamine, MEK inhibitors, MEK-162, methyltestosteron, methylprednisolone, MEDI-573, MEN-10755, MDX-H210, MDX-447, MDX-1379, MGV, midostaurin, minodronic acid, mitomycin, mivobulin, MK-2206, MK-0646 (dalotuzumab), MLN518, motexaf in gadolinium, MS-209, MS-275, MX6, neridronate, neratinib, Nexavar, neovastat, nilotinib, nimesulide, nitroglycerin, nolatrexed, norelin, N-acetylcysteine, 06-benzylguanine, oblimersen, omeprazole, oncophage, oncoVEXGM-CSF, ormiplatin, ortataxel, OX44 antibodies, OSI-027, OSI-906 (linsitinib), 4-1BB antibodies, oxantrazole, oestrogen, panitumumab, patupilone, PI3Ki, inhibitors, paxilisib, pegfilgrastim, PCK-3145, pegfilgrastim, PBI-1402, PBI-05204, PD0325901, PD-1 antibodies, PEG-paclitaxel, albumin-stabilized paclitaxel, PEP-005, PF-05197281, PF-05212384, PF-04691502, PHT-427, P-04, PKC412, P54, PI-88, pelitinib, pemetrexed, pentrix, perifosine, perillylalcohol, pertuzumab, PI3K inhibitors, PI3K/mTOR inhibitors, PG-TXL, PG2, PLX-4032/RO-5185426 (vemurafenib), PLX-3603/RO-5212054, PT-100, PWT-33597, PX-866, picoplatin, pivaloyloxymethylbutyrate, pixantrone, phenoxodiol O, PKI166, plevitrexed, plicamycin, polyprenic acid, porfiromycin, prednisone, prednisolone, quinamed, quinupristin, R115777, RAF-265, ramosetron, ranpirnase, RDEA-119/BAY 869766, RDEA-436, rebeccamycin analogues, receptor tyrosine kinase (RTK) inhibitors, revimid, RG-7167, RG-7304, RG-7421, RG-7321, RG 7440, rhizoxin, rhu-MAb, rinfabate, risedronate, rituximab, robatumumab, rofecoxib, RO-31-7453, RO-5126766, RO-5068760, RPR 109881A, rubidazone, rubitecan, R-flurbiprofen, RX-0201, S-9788, sabarubicin, SAHA, sargramostim, satraplatin, SB 408075, Se-015/Ve-015, SU5416, SU6668, SDX-101, semustin, seocalcitol, SM-11355, SN-38, SN-4071, SR-27897, SR-31747, SR-13668, SRL-172, sorafenib, spiroplatin, squalamine, suberanilohydroxamic acid, sutent, T 900607, T 138067, TAK-733, TAS-103, tacedinaline, talaporf in, Tarceva, tariquitar, tasisulam, taxotere, taxoprexin, tazarotene, tegafur, temozolamide, tesmilifene, testosterone, testosterone propionate, tesmilifene, tetraplatin, tetrodotoxin, tezacitabine, thalidomide, theralux, therarubicin, thymalfasin, thymectacin, tiazofurin, tipifarnib, tirapazamine, tocladesine, tomudex, toremofin, trabectedin, TransMID-107, transretinic acid, traszutumab, tremelimumab, tretinoin, triacetyluridine, triapine, triciribine, trimetrexate, TLK-286TXD 258, tykerb/tyverb, urocidin, valrubicin, vatalanib, vincristine, vinflunine, virulizin, WX-UK1, WX-554, vectibix, xeloda, XELOX, XL-147, XL-228, XL-281, XL-518/R-7420/GDC-0973, XL-765, YM-511, YM-598, ZD-4190, ZD-6474, ZD-4054, ZD-0473, ZD-6126, ZD-9331, ZD1839, ZSTK-474, zoledronat, zosuquidar, and combinations thereof.

In some embodiments, the at least one other therapeutic agent comprises a steroid. Steroids include, but are not limited to, dexamethasone, prednisolone, methyl prednisolone, prednisone, hydrocortisone, triamcinolone, betamethasone, and cortivazol. In some embodiments, the at least one other therapeutic agent comprises an anti-emetic. Anti-emetics include, but are not limited to, 5-HT3 receptor agonists (such as dolasetron, granisetron, ondansetron, tropisetron, palonosetron, and mirtazapine), dopamine agonists (such as domperidone, olanzapine, droperidol, haloperidol, chlorpromazine, prochlorperazine, alizapride, prochlorperazine, and metoclopramide), NK1 receptor antagonists (such as aprepitant and casopitant), antihistamines (such as cyclizine, diphenhydramine, dimenhydrinate, doxylamine, meclizine, promethazine, hydroxyzine), cannabinoids (such as cannabis, dronabinol, nabilone, and sativex), benzodiazepines (such as midazolam and lorazepam), anticholinergics (such as hyoscine), trimethobenzamide, ginger, emetrol, propofol, peppermint, muscimol, and ajwain.

In some embodiments, the at least one other therapeutic agent comprises anti-cancer agent which includes a mitotic inhibitor. In one embodiment, the mitotic inhibitor includes a taxane. In one embodiment, the mitotic inhibitor includes a taxane selected from the group consisting of paclitaxel and docetaxel.

In one embodiment, the pharmaceutical composition includes ONC-206 or a pharmaceutically acceptable salt thereof and at least one anti-cancer agent, wherein the anti-cancer agent includes, without limitation, one or more of acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bevacizumab, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, dromostanolone, duazomycin, edatrexate, eflomithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, fulvestrant, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine hydrochlride, megestrol, melengestrol acetate, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalampycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, verteporfin, vinblastine, vincristine sulfate, vindesine, vinepidine, vinglycinate, vinleurosine, vinorelbine, vinrosidine, vinzolidine, vorozole, zeniplatin, zinostatin, zoledronate, zorubicin and combinations thereof.

In one embodiment, the at least one additional therapeutic agent provides immunotherapy. In one embodiment, the at least one addtitional therapeutic agent is one or more checkdpoint inhibitor. In one embodiment, the at least one additional therapeutic agent is an adaptive cellular therapy.

In one embodiment, the at least one additional therapeutic agent is a device, such as a device that uses electric fields to disrupt cancer cell division, including technology referred to as tumor treating fields, also referred to a TTField, such as that provided by Novocure.

Examples of suitable anti-cancer agents as an additional therapeutic agent include, but are not limited to, one or more of Afinitor (Everolimus), Afinitor Disperz (Everolimus), Avastin (Bevacizumab), Bevacizumab, BiCNU (Carmustine), Carmustine, Carmustine Implant, Danyelza (Naxitamab-gqgk), Everolimus, Gliadel Wafer (Carmustine Implant), Lomustine, Mvasi (Bevacizumab), Naxitamab-gqgk, Temodar (Temozolomide), Temozolomide, and Zirabev (Bevacizumab).

In one embodiment, an additional therapeutic agent is two or more additional agents. As one example the additional therapeutic agent may be PCV, which is a combination of procarbazine hydrochloride, lomustine (gleostine), and vincristine sulfate.

Examples of suitable anti-cancer agents include, but are not limited to, those described Goodman and Gilman's The Pharmacological Basis of Therapeutics, 12th Ed., edited by Laurence Brunton, Bruce Chabner, Bjorn Knollman, McGraw Hill Professional, 2010.

In some exemplary embodiments, the pharmaceutical composition includes a salt (e.g., a mono- or di-salt) of ONC-206 and at least one other therapeutic agent, wherein the at least one other therapeutic agent comprises an anti-angiogenic agent. In one such embodiment, the anti-angiogenic agent is bevacizumab. In one embodiment, the anti-angiogenic agent is selected from the group consisting of aflibercept, axitinib, angiostatin, endostatin, 16 kDa prolactin fragment, laminin peptides, fibronectin peptides, tissue metalloproteinase inhibitors (TIMP 1, 2, 3, 4), plasminogen activator, inhibitors (PAI-1, -2), tumor necrosis factor α, (high dose, invitro), TGF-β1, interferons (IFN-α, -β, γ), ELR-CXC Chemokines: IL-12; SDF-1; MIG; platelet factor 4 (PF-4); IP-10, thrombospondin (TSP), SPARC, 2-methoxyoestradiol, proliferin-related protein, suramin, sorafenib, regorafenib, thalidomide, cortisone, linomide, fumagillin (AGM-1470; TNP-470), tamoxifen, retinoids, CM101, dexamethasone, leukemia inhibitoryfactor (LIF), hedgehog inhibitor and combinations thereof.

The pharmaceutical combination in accordance with the present disclosure can include the first and second therapeutic agents in any desired proportions provided that the synergistic or cooperative effect still occurs. The synergistic pharmaceutical combination in accordance with the present disclosure preferably contains the first and second therapeutic agents in a ratio of from about 1:9 to about 9:1. In one embodiment, the synergistic pharmaceutical combination pontains the first and second therapeutic agents in a ratio of from about 1:8 to about 8:1. In one embodiment, the synergistic pharmaceutical combination pontains the first and second therapeutic agents in a ratio of from about 1:7 to about 7:1. In one embodiment, the synergistic pharmaceutical combination pontains the first and second therapeutic agents in a ratio of from about 1:6 to about 6:1. In one embodiment, the synergistic pharmaceutical combination pontains the first and second therapeutic agents in a ratio of from about 1:5 to about 5:1. In one embodiment, the synergistic pharmaceutical combination pontains the first and second therapeutic agents in a ratio of from about 1:4 to about 4:1. In one embodiment, the synergistic pharmaceutical combination pontains the first and second therapeutic agents in a ratio of from about 1:3 to about 3:1. In one embodiment, the synergistic pharmaceutical combination pontains the first and second therapeutic agents in a ratio of from about 1:2 to about 2:1. In one embodiment, the synergistic pharmaceutical combination pontains the first and second therapeutic agents in a ratio of approximately 1:1.

In some prefered embodiments, the second therapeutic agent is selected from the group consisting of Allopurinol, Arsenic Trioxide, Azacitidine, Bortezomib, Bevacizumab, Capecitabine, Carboplatin, Celecoxib, Chlorambucil, Clofarabine, Cytarabine, Dacarbazine, Daunorubicin HCl, Docetaxel, Doxorubicin HCl, Floxuridine, Gemcitabine HCl, Hydroxyurea, Ifosfamide, Imatinib Mesylate, Ixabepilone, Lenalidomide, Megestrol acetate, Methotrexate, Mitotane, Mitoxantrone HCl, Oxaliplatin, Paclitaxel, Pralatrexate, Romidepsin, Sorafenib, Streptozocin, Tamoxifen Citrate, Topotecan HCl, Tretinoin, Vandetanib, Vismodegib, Vorinostat, and combinations thereof.

In some prefered embodiments, the second therapeutic agent comprises a small molecule multi-kinase inhibitor. In one embodiment, the small molecule multi-kinase inhibitor comprises sorafenib or regorafenib. In some prefered embodiments, the second therapeutic agent comprises a Hedgehog Pathway Inhibitor. In one prefered embodiment, the Hedgehog Pathway Inhibitor comprises vismodegib.

In some prefered embodiments, the second therapeutic agent include members of the classes drugs listed in the following Table A.

TABLE A
Second Therapeutic Agents

| Classes of | |
|---|---|
| drugs | Examples |
| Purine analogs | Examples include, but are not limited to, allopurinol, |
| | oxypurinol, clofarabine, and tisopurine |
| Pyrimidine | Examples include, but are not limited to, 5- |
| analogs | fluorouracil, Floxuridine (FUDR), capecitabine, |
| | cytarabine, 6-azauracil (6-AU), and gemcitabine |
| | (Gemzar). |
| Proteasome | Examples include, but are not limited to, bortezomib, |
| inhibitors | carfilzomib, cediranib, disulfiram, epigallocatechin-3- |
| | gallate, salinosporamide A, ONCX 0912, CEP-18770, |
| | MLN9708, epoxomicin, and MG132. |
| Anti- | Examples include, but are not limited to, bevacizumab, |
| angiogenic | aflibercept, sunitinib, sorafenib, pazopanib, |
| | vandetanib, cabozantinib, axitinib, ponatinib, |
| | regorafenib, ranibizumab, lapatinib, and vandetanib. |
| Platinum-based | Examples include, but are not limited to, cisplatin, |
| anti-neoplastic | carboplatin, oxaliplatin, satraplatin, picoplatin, |
| drugs | nedaplatin, and triplatin. |
| COX-2 | Examples include, but are not limited to, celecoxib, |
| inhibitors | valdecoxib (Bextra), parecoxib (Dynastat), lumiracoxib, |
| | etoricoxib, and rofecoxib. |
| Nitrogen | Examples include, but are not limited to, |
| mustards | cyclophosphamide, chlorambucil, uramustine, |
| | ifosfamide, melphalan, bendamustine, and mustine. |
| Alkylating | Examples include, but are not limited to, |
| agents | cyclophosphamide, mechlorethamine or mustine (HN2) |
| | (trade name Mustardgen), uramustine or uracil mustard, |
| | melphalan, chlorambucil, ifosfamide, bendamustine, |
| | carmustine, lomustine, streptozocin, and busulfan. |
| Anthracyclines | Examples include, but are not limited to, Daunorubicin |
| | (Daunomycin), Daunorubicin (liposomal), Doxorubicin |
| | (Adriamycin), Doxorubicin (liposomal), Epirubicin, |
| | Idarubicin, Valrubicin, and Mitoxantrone. |
| Taxanes | Examples include, but are not limited to, Paclitaxel |
| | (Taxol), Docetaxel (Taxotere), and albumin-bound |
| | paclitaxel (Abraxane). |
| Nucleotide | Examples include, but are not limited to, methotrexate, |
| synthesis | pralatrexate, hydroxyurea, and 5-fluorodeoxyuridine, 3,4- |
| inhibitor | dihydroxybenzylamine. |
| Bcr-abl | Examples include, but are not limited to, imatinib, |
| inhibitors | nilotinib, dasatinib, bosutinib and ponatinib. |
| Other | Examples include, but are not limited to, arsenic trioxide, |
| | thalidomide, revlimid, and mitotane. |
| Topoisomerase | Examples include, but are not limited to, amsacrine, |
| inhibitor | etoposide, etoposide phosphate, teniposide, doxorubicin, |
| | Topotecan (Hycamtin), Irinotecan (CPT-11, Camptosar), |
| | Exatecan, Lurtotecan, ST 1481, CKD 602, ICRF-193, |
| | and genistein. |
| HDAC | Examples include, but are not limited to, Vorinostat |
| inhibitors | (SAHA), Romidepsin (Istodax), Panobinostat (LBH589), |
| | Valproic acid (as Mg valproate), Belinostat (PXD101), |
| | Mocetinostat (MGCD0103), Abexinostat (PCI-24781), |
| | Entinostat (MS-275), SB939, Resminostat (4SC-201), |
| | Givinostat, Quisinostat (JNJ-26481585), CUDC-101, |
| | AR-42, CHR-2845, CHR-3996, 4SC-202, CG200745, |
| | ACY-1215, ME-344, sulforaphane, Kevetrin, and |
| | ATRA. |
| Multi-kinase | Examples include, but are not limited to, sorafenib, |
| inhibitors | regorafenib, and vandetanib. |
| Hormone | Examples include, but are not limited to, tamoxifen, |
| therapies | toremifene, Arimidex (anastrozole), Aromasin |
| | (exemestane), Femara (letrozole), and Fulvestrant |
| | (Faslodex). |
| Hedgehog | Examples include, but are not limited to, vismodegib, |
| signaling | |
| Inhibitors | BMS-833923, IPI-926, LDE-225, PF-04449913, LEQ |
| | 506, and TAK-441. |

In some embodiments, the second therapeutic agent includes drugs that target tumor necrosis factor-related apoptosis-inducing ligand (TRAIL) receptors. In one embodiment, the second therapeutic agent includes a recombinant TRAIL or an agonistic antibody that activates one or more TRAIL receptors. In one embodiment, the second therapeutic agent includes one or more antibodies or recombinant TRAIL that activate signaling by DR4 and/or DR5. In one embodiment, the second therapeutic agent includes one or more of mapatumumab, lexatumumab, Apomab, AMG-655, LBY-135 and rhApo2L/TRAIL. In one embodiment, the second therapeutic agent includes an active agent selected from the group consisting of Camptothecin, 5-FU, capecitabine, cisplatin, doxorubicin, irinotecan, paclitaxel, cisplatin, bortezomib, BH3I-2, rituximab, radiation, triterpenoids, sorafenib, gemcitabine, HDAC inhibitors, carboplatin, T-101 (a gossypol derivate), ABT-263, ABT-737, and GX-15-070 (obatoclax), vorinostat, cetuximab, panitumumab, bevacizumab, ganitumab, interferon gamma, sorafenib, XIAP antagonists, Bcl-2 antagonists, and Smac mimetics.

### II. DOSE

In one embodiment, a pharmaceutical composition according to the disclosure comprises ONC-206 or a pharmaceutically acceptable salt thereof in a dose ranging from about 10 mg to about 2000 mg, where the weight can, in certain embodiments be based on ONC-206 in its free base form. In one embodiment, a patient is an adult and the dose is calculated accordingly. In one embodiment, the patient is pediatric and the dose is calculated accordingly. In one embodiment, a pharmaceutical composition according to the disclosure comprises ONC-206 or a pharmaceutically acceptable salt thereof in a dose ranging from about 25 mg to about 2000 mg, where the weight can, in certain embodiments be based on ONC-206 in its free base form. In one embodiment, a pharmaceutical composition according to the disclosure comprises ONC-206 or a pharmaceutically acceptable salt thereof in a dose ranging from about 50 mg to about 2000 mg, where the weight can, in certain embodiments be based on ONC-206 in its free base form. In one embodiment, a pharmaceutical composition according to the disclosure comprises ONC-206 or a pharmaceutically acceptable salt thereof in a dose ranging from about 60 mg to about 2000 mg, where the weight can, in certain embodiments be based on ONC-206 in its free base form. In one embodiment, a pharmaceutical composition according to the disclosure comprises ONC-206 or a pharmaceutically acceptable salt thereof in a dose level selected for oral dosing from the group consisting of from about 10 mg to about 200 mg, from about 10 mg to about 300 mg, from about 10 mg to about 400 mg, from about 10 mg to about 500 mg, from about 10 mg to about 600 mg, from about 10 mg to about 700 mg, from about 10 mg to about 800 mg, from about 10 mg to about 900 mg, from about 10 mg to about 1000 mg, from about 10 mg to about 1100 mg, from about 10 mg to about 1200 mg, from about 10 mg to about 1300 mg, from about 10 mg to about 1400 mg, from about 10 mg to about 1500 mg, from about 10 mg to about 1600 mg, from about 10 mg to about 1700 mg, from about 10 mg to about 1800 mg, and from about 10 mg to about 1900 mg, and from about 10 mg to about 2000 mg. In one embodiment, a pharmaceutical composition according to the disclosure comprises ONC-206 or a pharmaceutically acceptable salt thereof in a dose level selected from the group consisting of from about 15 mg to about 200 mg, from about 15 mg to about 300 mg, from about 15 mg to about 400 mg, from about 15 mg to about 500 mg, from about 15 mg to about 600 mg, from about 15 mg to about 700 mg, from about 15 mg to about 800 mg, from about 15 mg to about 900 mg, from about 15 mg to about 1000 mg, from about 15 mg to about 1100 mg, from about 15 mg to about 1200 mg, from about 15 mg to about 1300 mg, from about 15 mg to about 1400 mg, from about 15 mg to about 1500 mg, from about 15 mg to about 1600 mg, from about 15 mg to about 1700 mg, from about 15 mg to about 1800 mg, and from about 15 mg to about 1900 mg, and from about 15 mg to about 2000 mg. In one embodiment, a pharmaceutical composition according to the disclosure comprises ONC-206 or a pharmaceutically acceptable salt thereof in a dose level selected from the group consisting of from about 20 mg to about 200 mg, from about 20 mg to about 300 mg, from about 20 mg to about 400 mg, from about 20 mg to about 500 mg, from about 20 mg to about 600 mg, from about 20 mg to about 700 mg, from about 20 mg to about 800 mg, from about 20 mg to about 900 mg, from about 20 mg to about 1000 mg, from about 20 mg to about 1100 mg, from about 20 mg to about 1200 mg, from about 20 mg to about 1300 mg, from about 20 mg to about 1400 mg, from about 20 mg to about 1500 mg, from about 20 mg to about 1600 mg, from about 20 mg to about 1700 mg, from about 20 mg to about 1800 mg, and from about 20 mg to about 1900 mg, and from about 20 mg to about 2000 mg. In one embodiment, a pharmaceutical composition according to the disclosure comprises ONC-206 or a pharmaceutically acceptable salt thereof in a dose level selected from the group consisting of from about 25 mg to about 200 mg, from about 25 mg to about 300 mg, from about 25 mg to about 400 mg, from about 25 mg to about 500 mg, from about 25 mg to about 600 mg, from about 25 mg to about 700 mg, from about 25 mg to about 800 mg, from about 25 mg to about 900 mg, from about 25 mg to about 1000 mg, from about 25 mg to about 1100 mg, from about 25 mg to about 1200 mg, from about 25 mg to about 1300 mg, from about 25 mg to about 1400 mg, from about 25 mg to about 1500 mg, from about 25 mg to about 1600 mg, from about 25 mg to about 1700 mg, from about 25 mg to about 1800 mg, from about 25 mg to about 1900 mg, and from about 25 mg to 2000 mg. In one embodiment, a pharmaceutical composition according to the disclosure comprises ONC-206 or a pharmaceutically acceptable salt thereof in a dose level selected from the group consisting of from about 30 mg to about 200 mg, from about 30 mg to about 300 mg, from about 30 mg to about 400 mg, from about 30 mg to about 500 mg, from about 30 mg to about 600 mg, from about 30 mg to about 700 mg, from about 30 mg to about 800 mg, from about 30 mg to about 900 mg, from about 30 mg to about 1000 mg, from about 30 mg to about 1100 mg, from about 30 mg to about 1200 mg, from about 30 mg to about 1300 mg, from about 30 mg to about 1400 mg, from about 30 mg to about 30 mg, from about 30 mg to about 1600 mg, from about 30 mg to about 1700 mg, from about 30 mg to about 1800 mg, and from about 30 mg to about 1900 mg based on ONC-206 in its free base form. In one embodiment, a pharmaceutical composition according to the disclosure comprises ONC-206 or a pharmaceutically acceptable salt thereof in a dose level selected from the group consisting of from about 35 mg to about 200 mg, from about 35 mg to about 300 mg, from about 35 mg to about 400 mg, from about 35 mg to about 500 mg, from about 35 mg to about 600 mg, from about 35 mg to about 700 mg, from about 35 mg to about 800 mg, from about 35 mg to about 900 mg, from about 35 mg to about 1000 mg, from about 35 mg to about 1100 mg, from about 35 mg to about 1200 mg, from about 35 mg to about 1300 mg, from about 35 mg to about 1400 mg, from about 35 mg to about 1500 mg, from about 35 mg to about 1600 mg, from about 35 mg to about 1700 mg, from about 35 mg to about 1800 mg, and from about 35 mg to about 1900 mg, and from about 35 mg to about 2000 mg. All values are based on ONC-206 in its free base form. In one embodiment, a pharmaceutical composition according to the disclosure comprises ONC-206 or a pharmaceutically acceptable salt thereof in a dose level selected from the group consisting of from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, and from about 95 mg to about 100 mg.

In one embodiment, a pharmaceutical composition according to the disclosure comprises ONC-206 or a pharmaceutically acceptable salt thereof in a dose ranging from about 0.10 mg/kg to about 40 mg/kg. In one embodiment, a pharmaceutical composition according to the disclosure comprises ONC-206 or a pharmaceutically acceptable salt thereof in a dose level selected from the group consisting of from about 0.10 mg/Kg to about 40 mg/Kg, about 0.2 mg/Kg to about 40 mg/Kg, about 0.3 mg/Kg to about 40 mg/Kg, about 0.4 mg/Kg to about 40 mg/Kg, about 0.5 mg/Kg to about 40 mg/Kg, about 0.6 mg/Kg to about 40 mg/Kg, about 0.7 mg/Kg to about 40 mg/Kg, about 0.8 mg/Kg to about 40 mg/Kg, about 0.9 mg/Kg to about 40 mg/Kg, about 1 mg/Kg to about 40 mg/Kg, from about 2 mg/Kg to about 40 mg/Kg, from about 3 mg/Kg to about 40 mg/Kg, from about 4 mg/Kg to about 40 mg/Kg, from about 5 mg/Kg to about 40 mg/Kg, from about 6 mg/Kg to about 40 mg/Kg, from about 7 mg/Kg to about 40 mg/Kg, from about 8 mg/Kg to about 40 mg/Kg, from about 9 mg/Kg to about 40 mg/Kg, from about 10 mg/Kg to about 40 mg/Kg, from about 11 mg/Kg to about 40 mg/Kg, from about 12 mg/Kg to about 40 mg/Kg, from about 13 mg/Kg to about 40 mg/Kg, from about 14 mg/Kg to about 40 mg/Kg, from about 15 mg/Kg to about 40 mg/Kg, from about 16 mg/Kg to about 40 mg/Kg, from about 17 mg/Kg to about 40 mg/Kg, from about 18 mg/Kg to about 40 mg/Kg, from about 19 mg/Kg to about 40 mg/Kg, from about 20 mg/Kg to about 40 mg/Kg, from about 21 mg/Kg to about 40 mg/Kg, from about 22 mg/Kg to about 40 mg/Kg, from about 23 mg/Kg to about 40 mg/Kg, from about 24 mg/Kg to about 40 mg/Kg, from about 25 mg/Kg to about 40 mg/Kg, from about 26 mg/Kg to about 40 mg/Kg, from about 27 mg/Kg to about 40 mg/Kg, from about 28 mg/Kg to about 40 mg/Kg, from about 29 mg/Kg to about 40 mg/Kg, from about 30 mg/Kg to about 40 mg/Kg, from about 31 mg/Kg to about 40 mg/Kg, from about 32 mg/Kg to about 40 mg/Kg, from about 33 mg/Kg to about 40 mg/Kg, from about 34 mg/Kg to about 40 mg/Kg, from about 35 mg/Kg to about 40 mg/Kg, from about 36 mg/Kg to about 40 mg/Kg, from about 37 mg/Kg to about 40 mg/Kg, from about 38 mg/Kg to about 40 mg/Kg, and from about 39 mg/Kg to about 40 mg/Kg.

In one embodiment, a pharmaceutical composition according to the disclosure comprises ONC-206 or a pharmaceutically acceptable salt thereof in a dose level selected from the group consisting of from about 1 mg/Kg to about 30 mg/Kg, from about 2 mg/Kg to about 30 mg/Kg, from about 3 mg/Kg to about 30 mg/Kg, from about 4 mg/Kg to about 30 mg/Kg, from about 5 mg/Kg to about 30 mg/Kg, from about 6 mg/Kg to about 30 mg/Kg, from about 7 mg/Kg to about 30 mg/Kg, from about 8 mg/Kg to about 30 mg/Kg, from about 9 mg/Kg to about 30 mg/Kg, from about 10 mg/Kg to about 30 mg/Kg, from about 11 mg/Kg to about 30 mg/Kg, from about 12 mg/Kg to about 30 mg/Kg, from about 13 mg/Kg to about 30 mg/Kg, from about 14 mg/Kg to about 30 mg/Kg, from about 15 mg/Kg to about 30 mg/Kg, from about 16 mg/Kg to about 30 mg/Kg, from about 17 mg/Kg to about 30 mg/Kg, from about 18 mg/Kg to about 30 mg/Kg, from about 19 mg/Kg to about 30 mg/Kg, from about 20 mg/Kg to about 30 mg/Kg, from about 21 mg/Kg to about 30 mg/Kg, from about 22 mg/Kg to about 30 mg/Kg, from about 23 mg/Kg to about 30 mg/Kg, from about 24 mg/Kg to about 30 mg/Kg, from about 25 mg/Kg to about 30 mg/Kg, from about 26 mg/Kg to about 30 mg/Kg, from about 27 mg/Kg to about 30 mg/Kg, from about 28 mg/Kg to about 30 mg/Kg, and from about 29 mg/Kg to about 30 mg/Kg.

In one embodiment, a pharmaceutical composition according to the disclosure comprises ONC-206 or a pharmaceutically acceptable salt thereof in a dose level selected from the group consisting of from about 1 mg/Kg to about 20 mg/Kg, from about 2 mg/Kg to about 20 mg/Kg, from about 3 mg/Kg to about 20 mg/Kg, from about 4 mg/Kg to about 20 mg/Kg, from about 5 mg/Kg to about 20 mg/Kg, from about 6 mg/Kg to about 20 mg/Kg, from about 7 mg/Kg to about 20 mg/Kg, from about 8 mg/Kg to about 20 mg/Kg, from about 9 mg/Kg to about 20 mg/Kg, from about 10 mg/Kg to about 20 mg/Kg, from about 11 mg/Kg to about 20 mg/Kg, from about 12 mg/Kg to about 20 mg/Kg, from about 13 mg/Kg to about 20 mg/Kg, from about 14 mg/Kg to about 20 mg/Kg, from about 15 mg/Kg to about 20 mg/Kg, from about 16 mg/Kg to about 20 mg/Kg, from about 17 mg/Kg to about 20 mg/Kg, from about 18 mg/Kg to about 20 mg/Kg, and from about 19 mg/Kg to about 20 mg/Kg.

In one embodiment, a pharmaceutical composition according to the disclosure comprises ONC-206 or a pharmaceutically acceptable salt thereof in a dose level selected from the group consisting of from about 1 mg/Kg to about 10 mg/Kg, from about 2 mg/Kg to about 10 mg/Kg, from about 3 mg/Kg to about 10 mg/Kg, from about 4 mg/Kg to about 10 mg/Kg, from about 5 mg/Kg to about 10 mg/Kg, from about 6 mg/Kg to about 10 mg/Kg, from about 7 mg/Kg to about 10 mg/Kg, from about 8 mg/Kg to about 10 mg/Kg, and from about 9 mg/Kg to about 10 mg/Kg.

In one embodiment, a pharmaceutical composition according to the disclosure comprises ONC-206 or a pharmaceutically acceptable salt thereof in a dose level ranging from about 12.5 mg/m² to about 1500 mg/m². In one embodiment, a pharmaceutical composition according to the disclosure comprises ONC-206 or a pharmaceutically acceptable salt thereof in a dose level selected from the group consisting of from about 15 mg/m² to about 1500 mg/m², from about 20 mg/m² to about 1500 mg/m², from about 25 mg/m² to about 1500 mg/m², from about 30 mg/m² to about 1500 mg/m², from about 35 mg/m² to about 1500 mg/m², from about 40 mg/m² to about 1500 mg/m², from about 45 mg/m² to about 1500 mg/m², from about 50 mg/m² to about 1500 mg/m², from about 55 mg/m² to about 1500 mg/m², from about 60 mg/m² to about 1500 mg/m², from about 65 mg/m² to about 1500 mg/m², from about 70 mg/m² to about 1500 mg/m², from about 75 mg/m² to about 1500 mg/m², from about 80 mg/m² to about 1500 mg/m², from about 85 mg/m² to about 1500 mg/m², from about 90 mg/m² to about 1500 mg/m², from about 95 mg/m² to about 1500 mg/m², from about 100 mg/m² to about 1500 mg/m², from about 105 mg/m² to about 1500 mg/m², from about 110 mg/m² to about 1500 mg/m², from about 115 mg/m² to about 1500 mg/m², from about 120 mg/m² to about 1500 mg/m², from about 125 mg/m² to about 1500 mg/m², from about 130 mg/m² to about 1500 mg/m², from about 135 mg/m² to about 1500 mg/m², from about 140 mg/m² to about 1500 mg/m², from about 145 mg/m² to about 1500 mg/m², from about 150 mg/m² to about 1500 mg/m², from about 155 mg/m² to about 1500 mg/m², from about 160 mg/m² to about 1500 mg/m², from about 165 mg/m² to about 1500 mg/m², from about 170 mg/m² to about 1500 mg/m², from about 175 mg/m² to about 1500 mg/m², from about 180 mg/m² to about 1500 mg/m², from about 185 mg/m² to about 1500 mg/m², from about 190 mg/m² to about 1500 mg/m², from about 195 mg/m² to about 1500 mg/m², from about 200 mg/m² to about 1500 mg/m², from about 205 mg/m² to about 1500 mg/m², from about 210 mg/m² to about 1500 mg/m², from about 215 mg/m² to about 1500 mg/m², from about 220 mg/m² to about 1500 mg/m², from about 225 mg/m² to about 1500 mg/m², from about 230 mg/m² to about 1500 mg/m², from about 235 mg/m² to about 1500 mg/m², from about 240 mg/m² to about 1500 mg/m², from about 245 mg/m² to about 1500 mg/m², from about 250 mg/m² to about 1500 mg/m², from about 255 mg/m² to about 1500 mg/m², from about 260 mg/m² to about 1500 mg/m², from about 265 mg/m² to about 1500 mg/m², from about 270 mg/m² to about 1500 mg/m², from about 275 mg/m² to about 1500 mg/m², from about 280 mg/m² to about 1500 mg/m², from about 285 mg/m² to about 1500 mg/m², from about 290 mg/m² to about 1500 mg/m², from about 295 mg/m² to about 1500 mg/m², from about 300 mg/m² to about 1500 mg/m², from about 305 mg/m² to about 1500 mg/m², from about 310 mg/m² to about 1500 mg/m², from about 315 mg/m² to about 1500 mg/m², from about 320 mg/m² to about 1500 mg/m², from about 325 mg/m² to about 1500 mg/m², from about 330 mg/m² to about 1500 mg/m², from about 335 mg/m² to about 1500 mg/m², from about 340 mg/m² to about 1500 mg/m², from about 345 mg/m² to about 1500 mg/m², from about 350 mg/m² to about 1500 mg/m², from about 355 mg/m² to about 1500 mg/m², from about 360 mg/m² to about 1500 mg/m², from about 365 mg/m² to about 1500 mg/m², from about 370 mg/m² to about 1500 mg/m², from about 375 mg/m² to about 1500 mg/m², from about 380 mg/m² to about 1500 mg/m², from about 385 mg/m² to about 1500 mg/m², from about 390 mg/m² to about 1500 mg/m², from about 395 mg/m² to about 1500 mg/m², from about 400 mg/m² to about 1500 mg/m², from about 405 mg/m² to about 1500 mg/m², from about 410 mg/m² to about 1500 mg/m², from about 415 mg/m² to about 1500 mg/m², from about 420 mg/m² to about 1500 mg/m², from about 425 mg/m² to about 1500 mg/m², from about 430 mg/m² to about 1500 mg/m², from about 435 mg/m² to about 1500 mg/m², from about 440 mg/m² to about 1500 mg/m², from about 445 mg/m² to about 1500 mg/m², from about 450 mg/m² to about 1500 mg/m², from about 455 mg/m² to about 1500 mg/m², from about 460 mg/m² to about 1500 mg/m², from about 465 mg/m² to about 1500 mg/m², from about 470 mg/m² to about 1500 mg/m², from about 475 mg/m² to about 1500 mg/m², from about 480 mg/m² to about 1500 mg/m², from about 485 mg/m² to about 1500 mg/m², from about 490 mg/m² to about 1500 mg/m², from about 495 mg/m² to about 1500 mg/m², from about 500 mg/m² to about 1500 mg/m², from about 505 mg/m² to about 1500 mg/m², from about 510 mg/m² to about 1500 mg/m², from about 515 mg/m² to about 1500 mg/m², from about 520 mg/m² to about 1500 mg/m², from about 525 mg/m² to about 1500 mg/m², from about 530 mg/m² to about 1500 mg/m², from about 535 mg/m² to about 1500 mg/m², from about 540 mg/m² to about 1500 mg/m², from about 545 mg/m² to about 1500 mg/m², from about 550 mg/m² to about 1500 mg/m², from about 555 mg/m² to about 1500 mg/m², from about 560 mg/m² to about 1500 mg/m², from about 565 mg/m² to about 1500 mg/m², from about 570 mg/m² to about 1500 mg/m², from about 575 mg/m² to about 1500 mg/m², from about 580 mg/m² to about 1500 mg/m², from about 585 mg/m² to about 1500 mg/m², from about 590 mg/m² to about 1500 mg/m², from about 595 mg/m² to about 1500 mg/m², from about 600 mg/m² to about 1500 mg/m², from about 605 mg/m² to about 1500 mg/m², from about 610 mg/m² to about 1500 mg/m², from about 615 mg/m² to about 1500 mg/m², from about 620 mg/m² to about 1500 mg/m², from about 625 mg/m² to about 1500 mg/m², from about 630 mg/m² to about 1500 mg/m², from about 635 mg/m² to about 1500 mg/m², from about 640 mg/m² to about 1500 mg/m², from about 645 mg/m² to about 1500 mg/m², from about 650 mg/m² to about 1500 mg/m², from about 655 mg/m² to about 1500 mg/m², from about 660 mg/m² to about 1500 mg/m², from about 665 mg/m² to about 1500 mg/m², from about 670 mg/m² to about 1500 mg/m², from about 675 mg/m² to about 1500 mg/m², from about 680 mg/m² to about 1500 mg/m², from about 685 mg/m² to about 1500 mg/m², from about 690 mg/m² to about 1500 mg/m², from about 695 mg/m² to about 1500 mg/m², from about 700 mg/m² to about 1500 mg/m², from about 705 mg/m² to about 1500 mg/m², from about 710 mg/m² to about 1500 mg/m², from about 715 mg/m² to about 1500 mg/m², from about 720 mg/m² to about 1500 mg/m², from about 725 mg/m² to about 1500 mg/m², from about 730 mg/m² to about 1500 mg/m², from about 735 mg/m² to about 1500 mg/m², from about 740 mg/m² to about 1500 mg/m², from about 745 mg/m² to about 1500 mg/m², from about 750 mg/m² to about 1500 mg/m², from about 755 mg/m² to about 1500 mg/m², from about 760 mg/m² to about 1500 mg/m², from about 765 mg/m² to about 1500 mg/m², from about 770 mg/m² to about 1500 mg/m², from about 775 mg/m² to about 1500 mg/m², from about 780 mg/m² to about 1500 mg/m², from about 785 mg/m² to about 1500 mg/m², from about 790 mg/m² to about 1500 mg/m², from about 795 mg/m² to about 1500 mg/m², from about 800 mg/m² to about 1500 mg/m², from about 805 mg/m² to about 1500 mg/m², from about 810 mg/m² to about 1500 mg/m², from about 815 mg/m² to about 1500 mg/m², from about 820 mg/m² to about 1500 mg/m², from about 825 mg/m² to about 1500 mg/m², from about 830 mg/m² to about 1500 mg/m², from about 835 mg/m² to about 1500 mg/m², from about 840 mg/m² to about 1500 mg/m², from about 845 mg/m² to about 1500 mg/m², from about 850 mg/m² to about 1500 mg/m², from about 855 mg/m² to about 1500 mg/m², from about 860 mg/m² to about 1500 mg/m², from about 865 mg/m² to about 1500 mg/m², from about 870 mg/m² to about 1500 mg/m², from about 875 mg/m² to about 1500 mg/m², from about 880 mg/m² to about 1500 mg/m², from about 885 mg/m² to about 1500 mg/m², from about 890 mg/m² to about 1500 mg/m², from about 895 mg/m² to about 1500 mg/m², from about 900 mg/m² to about 1500 mg/m², from about 905 mg/m² to about 1500 mg/m², from about 910 mg/m² to about 1500 mg/m², from about 915 mg/m² to about 1500 mg/m², from about 920 mg/m² to about 1500 mg/m², from about 925 mg/m² to about 1500 mg/m², from about 930 mg/m² to about 1500 mg/m², from about 935 mg/m² to about 1500 mg/m², from about 940 mg/m² to about 1500 mg/m², from about 945 mg/m² to about 1500 mg/m², from about 950 mg/m² to about 1500 mg/m², from about 955 mg/m² to about 1500 mg/m², from about 960 mg/m² to about 1500 mg/m², from about 965 mg/m² to about 1500 mg/m², from about 970 mg/m² to about 1500 mg/m², from about 975 mg/m² to about 1500 mg/m², from about 980 mg/m² to about 1500 mg/m², from about 985 mg/m² to about 1500 mg/m², from about 990 mg/m² to about 1500 mg/m², from about 995 mg/m² to about 1500 mg/m², from about 1000 mg/m² to about 1500 mg/m², from about 1005 mg/m² to about 1500 mg/m², from about 1010 mg/m² to about 1500 mg/m², from about 1015 mg/m² to about 1500 mg/m², from about 1020 mg/m² to about 1500 mg/m², from about 1025 mg/m² to about 1500 mg/m², from about 1030 mg/m² to about 1500 mg/m², from about 1035 mg/m² to about 1500 mg/m², from about 1040 mg/m² to about 1500 mg/m², from about 1045 mg/m² to about 1500 mg/m², from about 1050 mg/m² to about 1500 mg/m², from about 1055 mg/m² to about 1500 mg/m², from about 1060 mg/m² to about 1500 mg/m², from about 1065 mg/m² to about 1500 mg/m², from about 1070 mg/m² to about 1500 mg/m², from about 1075 mg/m² to about 1500 mg/m², from about 1080 mg/m² to about 1500 mg/m², from about 1085 mg/m² to about 1500 mg/m², from about 1090 mg/m² to about 1500 mg/m², from about 1095 mg/m² to about 1500 mg/m², from about 1100 mg/m² to about 1500 mg/m², from about 1105 mg/m² to about 1500 mg/m², from about 1110 mg/m² to about 1500 mg/m², from about 1115 mg/m² to about 1500 mg/m², from about 1120 mg/m² to about 1500 mg/m², from about 1125 mg/m² to about 1500 mg/m², from about 1130 mg/m² to about 1500 mg/m², from about 1135 mg/m² to about 1500 mg/m², from about 1140 mg/m² to about 1500 mg/m², from about 1145 mg/m² to about 1500 mg/m², from about 1150 mg/m² to about 1500 mg/m², from about 1155 mg/m² to about 1500 mg/m², from about 1160 mg/m² to about 1500 mg/m², from about 1165 mg/m² to about 1500 mg/m², from about 1170 mg/m² to about 1500 mg/m², from about 1175 mg/m² to about 1500 mg/m², from about 1180 mg/m² to about 1500 mg/m², from about 1185 mg/m² to about 1500 mg/m², from about 1190 mg/m² to about 1500 mg/m², from about 1195 mg/m² to about 1500 mg/m², from about 1200 mg/m² to about 1500 mg/m², from about 1205 mg/m² to about 1500 mg/m², from about 1210 mg/m² to about 1500 mg/m², from about 1215 mg/m² to about 1500 mg/m², from about 1220 mg/m² to about 1500 mg/m², from about 1225 mg/m² to about 1500 mg/m², from about 1230 mg/m² to about 1500 mg/m², from about 1235 mg/m² to about 1500 mg/m², from about 1240 mg/m² to about 1500 mg/m², from about 1245 mg/m² to about 1500 mg/m², from about 1250 mg/m² to about 1500 mg/m², from about 1255 mg/m² to about 1500 mg/m², from about 1260 mg/m² to about 1500 mg/m², from about 1265 mg/m² to about 1500 mg/m², from about 1270 mg/m² to about 1500 mg/m², from about 1275 mg/m² to about 1500 mg/m², from about 1280 mg/m² to about 1500 mg/m², from about 1285 mg/m² to about 1500 mg/m², from about 1290 mg/m² to about 1500 mg/m², from about 1295 mg/m² to about 1500 mg/m², from about 1300 mg/m² to about 1500 mg/m², from about 1305 mg/m² to about 1500 mg/m², from about 1310 mg/m² to about 1500 mg/m², from about 1315 mg/m² to about 1500 mg/m², from about 1320 mg/m² to about 1500 mg/m², from about 1325 mg/m² to about 1500 mg/m², from about 1330 mg/m² to about 1500 mg/m², from about 1335 mg/m² to about 1500 mg/m², from about 1340 mg/m² to about 1500 mg/m², from about 1345 mg/m² to about 1500 mg/m², from about 1350 mg/m² to about 1500 mg/m², from about 1355 mg/m² to about 1500 mg/m², from about 1360 mg/m² to about 1500 mg/m², from about 1365 mg/m² to about 1500 mg/m², from about 1370 mg/m² to about 1500 mg/m², from about 1375 mg/m² to about 1500 mg/m², from about 1380 mg/m² to about 1500 mg/m², from about 1385 mg/m² to about 1500 mg/m², from about 1390 mg/m² to about 1500 mg/m², from about 1395 mg/m² to about 1500 mg/m², from about 1400 mg/m² to about 1500 mg/m², from about 1405 mg/m² to about 1500 mg/m², from about 1410 mg/m² to about 1500 mg/m², from about 1415 mg/m² to about 1500 mg/m², from about 1420 mg/m² to about 1500 mg/m², from about 1425 mg/m² to about 1500 mg/m², from about 1430 mg/m² to about 1500 mg/m², from about 1435 mg/m² to about 1500 mg/m², from about 1440 mg/m² to about 1500 mg/m², from about 1445 mg/m² to about 1500 mg/m², from about 1450 mg/m² to about 1500 mg/m², from about 1455 mg/m² to about 1500 mg/m², from about 1460 mg/m² to about 1500 mg/m², from about 1465 mg/m² to about 1500 mg/m², from about 1470 mg/m² to about 1500 mg/m², from about 1475 mg/m² to about 1500 mg/m², from about 1480 mg/m² to about 1500 mg/m², from about 1485 mg/m² to about 1500 mg/m², from about 1490 mg/m² to about 1500 mg/m², and from about 1495 mg/m² to about 1500 mg/m².

### III. DOSAGE FORMS

Suitable pharmaceutical compositions for use with the methods of the present disclosure can be formulated into any dosage form that can be administered to a patient. In one embodiment, the pharmaceutical composition is in the form of an oral dosage unit or parenteral dosage unit. In one embodiment, the pharmaceutical composition is in the form of an oral dosage unit. In some embodiments, an oral dosage unit is fractionated into several, smaller doses, which are administered to a subject over a predetermined period of time in order to reduce toxicity of the therapeutic agent being administered. In some embodiments, an oral dosage unit is administered by a tablet or capsule comprising a controlled release formulation that can include a plurality of particles, granules, pellets, minitablets or tablets. In one embodiment, the pharmaceutical composition is in the form of a parenteral dosage unit. In one embodiment, the pharmaceutical composition is in the form of a parenteral dosage unit, wherein the parenteral dosage unit is selected from the group consisting of intravenous (IV), subcutaneous (SC), and intramuscular (M), rectal (PR) and transdermal dosage units. In one embodiment, the pharmaceutical composition is in a dosage form selected from the group consisting of sterile solutions, suspensions, suppositories, tablets and capsules. In one embodiment, the composition is an oral dosage form selected from the group consisting of a tablet, caplet, capsule, lozenge, syrup, liquid, suspension, and elixir, each of which includes a packaging configuration which allows reconstitution. In one embodiment, the composition is in an oral dosage form selected from the group consisting of tablets, hard shell capsules, soft gelatin capsules, beads, granules, aggregates, powders, gels, solids and semi-solids. In one embodiment, the composition is an oral dosage form comprising the compound of the present disclosure suspended in a liquid, such as water or a sports' drink, such as Gatorade®. The compound of the present disclosure, or a salt thereof, may be provided in a powder form for mixing with a liquid prior to administration to a patient in need thereof.

In some embodiments, suitable forms of pharmaceutical compositions for use in the methods of the present disclosure include dermatological compositions adapted for cutaneous topical administration. In some such embodiments, dermatological compositions include a cosmetically or pharmaceutically acceptable medium. In some embodiments, the dermatological compositions for topical administration can include ointments, lotions, creams, gels, drops, suppositories, sprays, liquids and powders. In some embodiments, conventional pharmaceutical carriers, aqueous, powder or oily bases, thickeners, skin enhancers and the like can be necessary or desirable and therefore can be used. Examples of suitable enhancers include, but are not limited to, ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin (U.S. Pat. No. 4,783,450); alcohols such as ethanol, propanol, octanol, benzyl alcohol, and the like; sugar alcohols or polyols such as mannitol, erythritol, lactitol, maltitol, sorbitol, xylitol, and the like; polyethylene glycol and esters thereof such as polyethylene glycol monolaurate; amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly citric acid and succinic acid. Azone® and sulfoxides such as DMSO and Ci0MSO may also be used, but are less preferred.

In some embodiments, the pharmaceutical composition of the present disclosure is in a dosage form selected from the group consisting of sustained release forms, controlled release forms, delayed release forms and response release forms.

### IV. METHODS OF USE

The compositions and methods of the present disclosure have utility in treating many disease conditions, including cancer (e.g., colorectal, brain, and glioblastoma). In one embodiment, the compositions and methods of the present disclosure are used to treat diseases such as ocular melanoma, desmoplastic round cell tumor, chondrosarcoma, leptomengial disease, diffuse large B-cell lymphoma, Acute Lymphoblastic Leukemia, Acute Myeloid Leukemia, Adrenocortical Carcinoma, AIDS-Related Cancers, AIDS-Related Lymphoma, Anal or Rectal Cancer, Appendix Cancer, Astrocytomas, and Atypical Teratoid/Rhabdoid Tumor. In one embodiment, the compositions and methods of the present disclosure are used to treat diseases such as Basal Cell Carcinoma, Basal Cell Nevus Syndrome, Gorlin-Nevus Syndrome, Bile Duct Cancer, Bladder Cancer, Bone Cancer, Osteosarcoma and Malignant Fibrous Histiocytoma, Brain Tumor, Breast Cance, Bronchial Tumors, Burkitt Lymphoma, and Spinal Cord Tumors. In one embodiment, the compositions and methods of the present disclosure are used to treat cdiseases such as Carcinoid Tumor, Carcinoma of Unknown Primary, Central Nervous System Atypical Teratoid/Rhabdoid Tumor, Leptomeningeal Disease, Central Nervous System Embryonal Tumors, Central Nervous System Lymphoma, Cervical Cancer, Chordoma, Chronic Lymphocytic Leukemia, Chronic Myelogenous Leukemia, Chronic Myeloproliferative Disorders, Colon Cancer, Colorectal Cancer, Craniopharyngioma, and Cutaneous T-Cell Lymphoma (including, but not limited to, Sezary syndrome and mycosis fungoides (MF)). In one embodiment, the compositions and methods of the present disclosure are used to treat cdiseases such as Embryonal Tumors of Central Nervous System, Endometrial Cancer, Ependymoblastoma, Ependymoma, Esophageal Cancer, Ewing Sarcoma Family of Tumors, Extracranial Germ Cell Tumor, Extragonadal Germ Cell Tumor, Extrahepatic Bile Duct Cancer, and Eye Cancer. In one embodiment, the compositions and methods of the present disclosure are used to treat cdiseases such as Gallbladder Cancer, Gastric (Stomach) Cancer, Gastrointestinal Carcinoid Tumor, Gastrointestinal Stromal Tumor (GIST), Germ Cell Tumor, Gestational Trophoblastic Tumor, and Glioma. In one embodiment, the compositions and methods of the present disclosure are used to treat cancer selected from the group consisting of Hairy Cell Leukemia, Head and Neck Cancer, Hepatocellular (Liver) Cancer, Histiocytosis, Hodgkin Lymphoma, and Hypopharyngeal Cancer. In one embodiment, the compositions and methods of the present disclosure are used to treat cdiseases such as Kaposi Sarcoma, and Kidney (Renal Cell) Cancer. In one embodiment, the compositions and methods of the present disclosure are used to treat diseases such as Langerhans Cell Histiocytosis, Laryngeal Cancer, Lip and Oral Cavity Cancer, Liver Cancer, Lung Cancer, Non-Hodgkin Lymphoma, and Primary Central Nervous System Lymphoma. In one embodiment, the compositions and methods of the present disclosure are used to treat diseases such as Waldenström's macroglobulinemia (lymphoplasmacytic lymphoma), Malignant Fibrous Histiocytoma of Bone and Osteosarcoma, Medulloblastoma, Medulloepithelioma, Melanoma, Merkel Cell Carcinoma, Mesothelioma, Metastatic Squamous Neck Cancer with Occult Primary, Multiple Endocrine Neoplasia Syndrome, Mouth Cancer, Multiple Myeloma/Plasma Cell Neoplasm, Mycosis Fungoides, Myelodysplastic Syndromes, Myelodysplastic/Myeloproliferative Neoplasms, Multiple Myeloma, and Myeloproliferative Disorders. In one embodiment, the compositions and methods of the present disclosure are used to treat cancer. In one embodiment, the compositions and methods of the present disclosure are used to treat diseases such as Nasal Cavity and Paranasal Sinus Cancer, Nasopharyngeal Cancer, and Neuroblastoma. In one embodiment, the compositions and methods of the present disclosure are used to treat diseases such as Oral Cancer, Lip and Oral Cavity Cancer, Oropharyngeal Cancer, Osteosarcoma and Malignant Fibrous Histiocytoma of Bone, Ovarian Cancer, Ovarian Germ Cell Tumor, Ovarian Epithelial Cancer, and Ovarian Low Malignant Potential Tumor. In one embodiment, the compositions and methods of the present disclosure are used to treat diseases such as Pancreatic Cancer, Papillomatosis, Paranasal Sinus and Nasal Cavity Cancer, Parathyroid Cancer, Penile Cancer, Pharyngeal Cancer, Pineal Parenchymal Tumors of Intermediate Differentiation, Pineoblastoma and Supratentorial Primitive Neuroectodermal Tumors, Pituitary Tumor, Pleuropulmonary Blastoma, Pregnancy and Breast Cancer, Primary Central Nervous System Lymphoma, and Prostate Cancer. In one embodiment, the compositions and methods of the present disclosure are used to treat cancer selected from the group consisting of Rectal Cancer, Renal Cell (Kidney) Cancer, Renal Pelvis and Ureter, Respiratory Tract Carcinoma Involving the NUT Gene on Chromosome 15, Retinoblastoma, and Rhabdomyosarcoma. In one embodiment, the compositions and methods of the present disclosure are used to treat high grade prostate cancer. In one embodiment, the compositions and methods of the present disclosure are used to treat medium grade prostate cancer. In one embodiment, the compositions and methods of the present disclosure are used to treat low grade prostate cancer. In one embodiment, the compositions and methods of the present disclosure are used to treat castration-resistant prostate cancer.

In one embodiment, the present use or method relates to the treatment of one or more adult central nervous system (CNS) tumors. An adult central nervous system tumor is a disease in which abnormal cells form in the tissues of the brain and/or spinal cord.

In one embodiment, the present use or method relates to the treatment of one or more pediatric central nervous system (CNS) tumors. A pediatric central nervous system tumor is a disease in which abnormal cells form in the tissues of the brain and/or spinal cord of a patient who is from the ages of about 0 to about 18 years of age.

A tumor that starts in another part of the body and spreads to the brain is called a metastatic brain tumor. There are different types of brain and spinal cord tumors for which the present compounds are believed to be therapeutically effective, whether alone or in combination with an additional therapeutic agent: Astrocytic Tumors, Oligodendroglial Tumors, Mixed Gliomas, Ependymal Tumors, Medulloblastomas, Pineal Parenchymal Tumors, Meningeal Tumors, Germ Cell Tumors, and Craniopharyngioma (Grade I). Certain genetic syndromes may increase the risk of a central nervous system tumor, and the present disclosure contemplates screening for such. Certain factors affect prognosis (chance of recovery) and treatment options and, similarly, the present disclosure contemplates screening for such.

In one embodiment, the compositions and methods of the present disclosure are used to treat a proliferative skin disorder. In one embodiment, the compositions and methods of the present disclosure are used to treat a proliferative skin disorder, wherein the proliferative skin disorder is psoriasis. In one embodiment, the compositions and methods of the present disclosure are used to treat cancer selected from the group consisting of Salivary Gland Cancer, Sarcoma, Sézary Syndrome, Skin Cancer, Ocular Cancer, Skin Carcinoma, Small Intestine Cancer, Soft Tissue Sarcoma, Squamous Cell Carcinom, Squamous Neck Cancer with Occult Primary, and Supratentorial Primitive Neuroectodermal Tumors. In one embodiment, the compositions and methods of the present disclosure are used to treat cancer selected from the group consisting of T-Cell Lymphoma, Testicular Cancer, Throat Cancer, Thymoma and Thymic Carcinoma, Thyroid Cancer, Transitional Cell Cancer of the Renal Pelvis and Ureter, and Gestational Trophoblastic Tumor. In one embodiment, the compositions and methods of the present disclosure are used to treat cancer selected from the group consisting of Carcinoma of Unknown Primary Site, Cancer of Unknown Primary Site, Unusual Cancers of Childhood, Transitional Cell Cancer Of the Renal Pelvis and Ureter, Urethral Cancer, and Uterine Sarcoma. In one embodiment, the compositions and methods of the present disclosure are used to treat cancer selected from the group consisting of Vaginal Cancer, and Vulvar Cancer. In one embodiment, the compositions and methods of the present disclosure are used to treat cancer selected from the group consisting of Wilms Tumor, and Women's Cancers.

In some embodiments, the compositions and methods of the present disclosure are used as a first-line therapy (sometimes called primary therapy). In some embodiments, the compositions and methods of the present disclosure are used as a second-line therapy. In some embodiments, the compositions and methods of the present disclosure are used as a third-line therapy. In some embodiments, the compositions and methods of the present disclosure are used as a salvage therapy. The term “salvage therapy” as used herein means a therapeutic agent that can be taken with any regimen after a subject's initial treatment regimen has failed or after the subject's condition has not responded to an initial treatment. In some embodiments, the compositions and methods of the present disclosure are used as a rescue therapy. In one embodiment of the rescue therapy, the compositions of the present disclosure are used as a rescue agent to counteract the action of an initial treatment. In one embodiment of the rescue therapy, the compositions of the present disclosure are used as rescue agent which is administered to a subject who has developed resistance to a standard or an initial treatment. In some embodiments, the compositions and methods of the present disclosure are used as a neoadjuvant therapy. In one embodiment, the neoadjuvant therapy comprises administration of one or more of the therapeutic agents of the present disclosure to a subject before a main or first line treatment. In one embodiment, the neoadjuvant therapy reduces the size or extent of the cancer being treated before a main or first line treatment is administered to the subject undergoing treament. In some embodiments, the compositions and methods of the present disclosure are used as an adjuvant therapy. In one embodiment, the adjuvant therapy comprises administration of one or more therapeutic agents of the present disclosure to a subject, wherein the one or more therapeutic agent that modify the effect of other therapeutic agents that are already administered to the subject or are concurrently administered to the subject or subsequently administered to the subject.

In some embodiments, the compositions and methods of the present disclosure exhibit reduced chance of drug-drug interactions. In some embodiments, the compositions and methods of the present disclosure, ONC-206 and/or a pharmaceutically acceptable salt thereof are eliminated from the patient's body before it can interact with another pharmaceutically active agent or therapy.

In some embodiments, the compositions and methods of the present disclosure, ONC-206 and/or a pharmaceutically acceptable salt thereof exhibit tonicity level that facilitates combinations with other pharmaceutical agents.

The utility of the methods and compositions of the present disclosure is not limited to any particular animal species. In one embodiment, a subject treated according to methods and using compositions of the present disclosure, can be mammalian or non-mammalian. In one embodiment, a mammalian subject can be any mammal including, but not limited to, a human; a non-human primate; a rodent such as a mouse, rat, or guinea pig; a domesticated pet such as a cat or dog; a horse, cow, pig, sheep, goat, or rabbit. In one embodiment, a non-mammalian subject can be any non-mammal including, but not limited to, a bird such as a duck, goose, chicken, or turkey. In one embodiment, subjects can be either gender and can be any age. The composition and methods can also be used to prevent cancer. The composition and methods can also be used to stimulate the immune system.

The utility of the methods and compositions of the present disclosure is not limited to any particular age of the subject. In one embodiment, a subject treated according to methods and using compositions of the present disclosure can be under the age of 5, 12, 16, or 18 (pediatrics), over the age of 18 years, over the age of 20 years, over the age of 25 years, over the age of 30 years, over the age of 35 years, over the age of 40 years, over the age of 45 years, over the age of 50 years, over the age of 55 years, over the age of 60 years, or over the age of 65 years. In one embodiment a subject treated according to methods and using compositions of the present disclosure can be under the age of 50 years, under the age of 55 years, under the age of 60 years, or under the age of 65 years.

In one embodiment the subject has received at least one prior therapeutic agent. In one embodiment the subject has received at least two, at least three, or at least four prior therapeutic agents. In one embodiment the prior therapeutic agent is ibrutinib, bortezomib, carfilzomib, temozolomide, bevacizumab, cyclophosphamide, hydroxydaunorubicin, vincristine, prednisone, cytarabine, cisplatin, rituximab, 5-fluorouracil, oxaliplatin, leucovorin, or lenalidomide.

In one embodiment the subject has been treated with one or more form of radiation. In one embodiment the subject has been treated with one or more form of surgery.

In some embodiments of the methods of treating a cancer, the cancer no longer responds to treatment with ibrutinib, bortezomib, carfilzomib, temozolomide, bevacizumab, cyclophosphamide, hydroxydaunorubicin, vincristine, prednisone, cytarabine, cisplatin, rituximab, 5-fluorouracil, oxaliplatin, leucovorin, lenalidomide, radiation, surgery, or a combination thereof.

In some embodiments, the compositions and methods of the present disclosure have dose response relation in cancer cells that is different from dose response relation of the same the compositions and methods in normal cells.

In some embodiments, the compositions and methods of the present disclosure have utility in treating cancer in a subject. In one embodiment, the compositions and methods of the present disclosure have utility in treating cancer in a human subject. In one embodiment, the method of treatment comprises administering to a subject in need of such treatment: (i) a first therapeutic agent including a compound comprising ONC-206 or a pharmaceutically acceptable salt thereof in combination with (ii) a second therapeutic agent, wherein the first therapeutic agent and the second therapeutic agent are administered either simultaneously or sequentially. The second therapeutic agent can be any suitable therapeutic agent, including any of the pharmaceutically active agents disclosed in in this application. In one embodiment, the pharmaceutically accetable salt of ONC-206 includes a di-hydrochloride salt.

It is understood that ONC-206 as the di-HCl or an alternative di-salt thereof apparent from the teaching of this disclosure, can be substitued for a ONC-206 in any of the compositions or dosing regimens described hererin.

In some embodiments, the method of treatment comprises administering to a subject in need of such treatment, a pharmaceutically effective amount of ONC-206 or a pharmaceutically acceptable salt thereof and a pharmaceutically acceptable carrier.

In some embodiments, the method of treatment of the present disclosure comprises administering a synergistic pharmaceutical combination, either simultaneously or sequentially, to a subject in need of such treatment, wherein the synergistic pharmaceutical combination comprising (i) a first therapeutic agent comprising ONC-206 or a pharmaceutically acceptable salt thereof; and (ii) a second therapeutic agent. In one embodiment, the method of treatment comprises administering to a subject in need of such treatment, either simultaneously or sequentially, therapeutically synergistic effective amounts of a first therapeutic agent comprising ONC-206 or a pharmaceutically acceptable salt thereof in combination with a second therapeutic agent. In one embodiment, the method of treatment comprises administering to a subject in need of such treatment an effective amount of a first therapeutic agent comprising ONC-206 or a pharmaceutically acceptable salt thereof in combination with an effective amount of a second therapeutic agent, wherein the combination provides a synergistic effect in the in vivo treatment of cancer sensitive to the combination, and wherein the first therapeutic agent and the second therapeutic agent are administered either simultaneously or sequentially. In one embodiment, the method of treatment comprises administering to a subject in need of such treatment an effective amount of a first therapeutic agent comprising ONC-206 or a pharmaceutically acceptable salt thereof in combination with an effective amount of a second therapeutic agent, wherein the combination provides a synergistic effect in the in vivo treatment of a minimal residual disease sensitive to the combination, and wherein the first therapeutic agent and the second therapeutic agent are administered either simultaneously or sequentially.

In some embodiments, the second drug can be given before or prior to ONC-206.

In one embodiment, the method of treatment of the present disclosure targets cancer, wherein the cancer is selected from the group consisting of solid tumors, liquid tumors, lymphomas, leukemias, or myelomas.

In one embodiment, the method of treatment of the present disclosure targets a solid tumor, wherein the solid tumor is selected from the group consisting of: Cervical Cancer, Endometrial Cancer, Extracranial Germ Cell Tumor; Extragonadal Germ Cell Tumor; Germ Cell Tumor; Gestational Trophoblastic Tumor; Ovarian Cancer, Ovarian Germ Cell Tumor, Ovarian Epithelial Cancer, and Ovarian Low Malignant Potential Tumor; Penile Cancer, Prostate Cancer; Pregnancy and Breast Cancer; high grade prostate cancer; medium grade prostate cancer; low grade prostate cancer; castration-resistant prostate cancer; Breast Cancer; Bile Duct Cancer; Extrahepatic Bile Duct Cancer; Gallbladder Cancer; Hepatocellular (Liver) Cancer; Kidney (Renal Cell) Cancer; Liver Cancer, Renal Cell (Kidney) Cancer, Renal Pelvis and Ureter; Basal Cell Carcinoma; Basal Cell Nevus Syndrome, Gorlin-Nevus Syndrome, Melanoma, Merkel Cell Carcinoma, Papillomatosis, Multiple Endocrine Neoplasia Syndrome; Pancreatic Cancer, Parathyroid Cancer, ocular melanoma; Eye Cancer; Retinoblastoma; Malignant Fibrous Histiocytoma; Ewing Sarcoma Family of Tumors; desmoplastic round cell tumor; chondrosarcoma, Kaposi Sarcoma, Rhabdomyosarcoma; Spinal Cord Tumors, Leptomeningeal Disease, Central Nervous System Embryonal Tumors, Chordoma, Embryonal Tumors of Central Nervous System, Ependymoblastoma, Ependymoma, Neuroblastoma; Pineal Parenchymal Tumors of Intermediate Differentiation, Pineoblastoma; Adrenocortical Carcinoma; Bone Cancer, Osteosarcoma; Malignant Fibrous Histiocytoma of Bone and Osteosarcoma; Osteosarcoma and Malignant Fibrous Histiocytoma of Bone; Carcinoid Tumor, Carcinoma of Unknown Primary, Bronchial Tumors, Lung Cancer, Pleuropulmonary Blastoma; Respiratory Tract Carcinoma Involving the NUT Gene on Chromosome 15, Astrocytomas, Atypical Teratoid/Rhabdoid Tumor; Central Nervous System Atypical Teratoid/Rhabdoid Tumor, Craniopharyngioma, Glioma, Brain cancer, Medulloblastoma, Medulloepithelioma, Supratentorial Primitive Neuroectodermal Tumors; Pituitary Tumor; Gastric (Stomach) Cancer, Gastrointestinal Carcinoid Tumor, Gastrointestinal Stromal Tumor (GIST), Bladder Cancer, Anal or Rectal Cancer, Appendix Cancer, Esophageal Cancer, Hypopharyngeal Cancer; Laryngeal Cancer, Lip and Oral Cavity Cancer, Metastatic Squamous Neck Cancer with Occult Primary, Mouth Cancer, Nasal Cavity and Paranasal Sinus Cancer, Nasopharyngeal Cancer, Oral Cancer, Lip and Oral Cavity Cancer, Oropharyngeal Cancer, Paranasal Sinus and Nasal Cavity Cancer, Pharyngeal Cancer; Head and Neck Cancer, and Mesothelioma.

In one embodiment, the method of treatment of the present disclosure targets lymphoma, wherein the lymphoma is selected from the group consisting of: diffuse large B-cell lymphoma, AIDS-Related Lymphoma, Cutaneous T-Cell Lymphoma, Sezary syndrome, mycosis fungoides (MF); Histiocytosis; Burkitt Lymphoma, and Central Nervous System Lymphoma; Non-Hodgkin Lymphoma, and Primary Central Nervous System Lymphoma, Hodgkin Lymphoma, Waldenström's macroglobulinemia; Mycosis Fungoides; Primary Central Nervous System Lymphoma; lymphoplasmacytic lymphoma, and Primary Central Nervous System Lymphoma.

In one embodiment, the method of treatment of the present disclosure targets Non-Hodgkin's lymphoma (NHL), wherein the Non-Hodgkin's lymphoma is selected from the group consisting of mantle cell lymphoma, diffuse large B-cell lymphoma, follicular lymphoma, marginal zone lymphoma, small lymphocytic lymphoma, lyphoplasmacytic NHL, Waldenstrom's macroglobulinaemia, and skin lymphomas.

In one embodiment, the method of treatment of the present disclosure targets leukemia, wherein the leukemia is selected from the group consisting of: Acute Lymphoblastic Leukemia (ALL), Chronic Lymphocytic Leukemia (CLL), Chronic Myeloproliferative Disorders; Hairy Cell Leukemia; Acute Myeloid Leukemia (AML); Chronic Myelogenous Leukemia (CML); and Langerhans Cell Histiocytosis.

In one embodiment, the method of treatment of the present disclosure targets acute leukemia, wherein the acute leukemia is selected from the group consisting of acute lymphotyte leukemia, acute myeloid leukemia, chronic lymphoblasitc leukemia, chronic myeloid leukemia, myelodysplastic syndrome, or myeloproliferative disease.

In one embodiment, the method of treatment of the present disclosure targets myeloma, wherein the myeloma is selected from the group consisting of: IgA myeloma; IgG myeloma; IgM myeloma; IgD myeloma; IgE myeloma; light chain myeloma; non secretory myeloma; Multiple Myeloma/Plasma Cell Neoplasm, Multiple Myeloma, Myelodysplastic Syndromes, Myelodysplastic/Myeloproliferative Neoplasms, Myeloproliferative Disorders.

In one embodiment, the method of treatment of the present disclosure targets cancer, wherein the cancer is selected from the group consisting of Acute Lymphoblastic Leukemia, Acute Myeloid Leukemia, Adrenocortical Carcinoma, AIDS-Related Cancers, AIDS-Related Lymphoma, Anal or Rectal Cancer, Appendix Cancer, Astrocytomas, and Atypical Teratoid/Rhabdoid Tumor.

In one embodiment, the method of treatment of the present disclosure targets cancer, wherein the cancer is selected from the group consisting of Basal Cell Carcinoma, Basal Cell Nevus Syndrome, Gorlin-Nevus Syndrome, Bile Duct Cancer, Bladder Cancer, Bone Cancer, Osteosarcoma and Malignant Fibrous Histiocytoma, Brain Tumor, Breast Cance, Bronchial Tumors, Burkitt Lymphoma, and Spinal Cord Tumors.

In one embodiment, the method of treatment of the present disclosure targets cancer, wherein the cancer is selected from the group consisting of Carcinoid Tumor, Carcinoma of Unknown Primary, Central Nervous System Atypical Teratoid/Rhabdoid Tumor, Central Nervous System Embryonal Tumors, Central Nervous System Lymphoma, Cervical Cancer, Chordoma, Chronic Lymphocytic Leukemia, Chronic Myelogenous Leukemia, Chronic Myeloproliferative Disorders, Colon Cancer, Colorectal Cancer, Craniopharyngioma, and Cutaneous T-Cell Lymphoma (including, but not limited to Sezary syndrome and mycosis fungoides).

In one embodiment, the method of treatment of the present disclosure targets cancer, wherein the cancer is selected from the group consisting of Embryonal Tumors of Central Nervous System, Endometrial Cancer, Ependymoblastoma, Ependymoma, Esophageal Cancer, Ewing Sarcoma Family of Tumors, Desmoplastic Round Cell Tumor, Chondrosarcoma, Extracranial Germ Cell Tumor, Extragonadal Germ Cell Tumor, Extrahepatic Bile Duct Cancer, and Eye Cancer, including Intraocular Melanoma and Retinoblastoma.

In one embodiment, the method of treatment of the present disclosure targets cancer, wherein the cancer is selected from the group consisting of Gallbladder Cancer, Gastric (Stomach) Cancer, Gastrointestinal Carcinoid Tumor, Gastrointestinal Stromal Tumor (GIST), Germ Cell Tumor, Gestational Trophoblastic Tumor, and Glioma.

In one embodiment, the method of treatment of the present disclosure targets cancer, wherein the cancer is selected from the group consisting of Hairy Cell Leukemia, Head and Neck Cancer, Hepatocellular (Liver) Cancer, Histiocytosis, Hodgkin Lymphoma, and Hypopharyngeal Cancer.

In one embodiment, the method of treatment of the present disclosure targets cancer, wherein the cancer is selected from the group consisting of Kaposi Sarcoma, and Kidney (Renal Cell) Cancer.

The method of treating cancer according to claim 1, wherein the cancer is selected from the group consisting of Langerhans Cell Histiocytosis, Laryngeal Cancer, Lip and Oral Cavity Cancer, Liver Cancer, Lung Cancer, including Non-Small Cell Lung Cancer, and Small Cell Lung Cance, Non-Hodgkin Lymphoma, and Primary Central Nervous System Lymphoma.

In one embodiment, the method of treatment of the present disclosure targets cancer, wherein the cancer is selected from the group consisting of Waldenström's macroglobulinemia (lymphoplasmacytic lymphoma), Malignant Fibrous Histiocytoma of Bone and Osteosarcoma, Medulloblastoma, Medulloepithelioma, Melanoma, Merkel Cell Carcinoma, Mesothelioma, Metastatic Squamous Neck Cancer with Occult Primary, Multiple Endocrine Neoplasia Syndrome, Mouth Cancer, Multiple Myeloma/Plasma Cell Neoplasm, Mycosis Fungoides, Myelodysplastic Syndromes, Myelodysplastic/Myeloproliferative Neoplasms, Multiple Myeloma, and Myeloproliferative Disorders.

In one embodiment, the method of treatment of the present disclosure is useful for treating cancer, wherein the cancer is selected from the group consisting of Nasal Cavity and Paranasal Sinus Cancer, Nasopharyngeal Cancer, and Neuroblastoma.

In one embodiment, the method of treatment of the present disclosure is useful for treating a neuroendocrine tumor, including one or more of an adrenal cancer, adrenal cortical carcinoma, desmoplastic small round cell tumors (DSRTCs), small cell lung cancer, neuroendocrine prostate cancer, carcinoid tumors, Merkel cell carcinoma, pancreatic neuroendocrine tumors, paraganglioma, and pheochromocytoma. In one aspect, the tumor may be one or more of pheochromocytoma, paraganglioma, adrenal cortical carcinoma, DSRTC, small cell lung cancer, and neuroendocrine prostate cancer

In one embodiment, the method of treatment of the present disclosure is useful for treating cancer, wherein the cancer is selected from the group consisting of Oral Cancer, Lip and Oral Cavity Cancer, Oropharyngeal Cancer, Osteosarcoma and Malignant Fibrous Histiocytoma of Bone, Ovarian Cancer, Ovarian Germ Cell Tumor, Ovarian Epithelial Cancer, and Ovarian Low Malignant Potential Tumor.

In one embodiment, the method of treatment of the present disclosure is useful for treating cancer, wherein the cancer is selected from the group consisting of Pancreatic Cancer, Papillomatosis, Paranasal Sinus and Nasal Cavity Cancer, Parathyroid Cancer, Penile Cancer, Pharyngeal Cancer, Pineal Parenchymal Tumors of Intermediate Differentiation, Pineoblastoma and Supratentorial Primitive Neuroectodermal Tumors, Pituitary Tumor, Pleuropulmonary Blastoma, Pregnancy and Breast Cancer, Primary Central Nervous System Lymphoma, and Prostate Cancer.

In one embodiment, the method of treatment of the present disclosure is useful for treating cancer, wherein the cancer is selected from the group consisting of Rectal Cancer, Renal Cell (Kidney) Cancer, Renal Pelvis and Ureter, Respiratory Tract Carcinoma Involving the NUT Gene on Chromosome 15, Retinoblastoma, and Rhabdomyosarcoma.

In one embodiment, the method of treatment of the present disclosure is useful for treating cancer, wherein the cancer is selected from the group consisting of Salivary Gland Cancer, Sarcoma, Sézary Syndrome, Skin Cancer, Skin Carcinoma, Small Intestine Cancer, Soft Tissue Sarcoma, Squamous Cell Carcinom, Squamous Neck Cancer with Occult Primary, and Supratentorial Primitive Neuroectodermal Tumors.

In one embodiment, the method of treatment of the present disclosure is useful for treating cancer, wherein the cancer is selected from the group consisting of T-Cell Lymphoma, Testicular Cancer, Throat Cancer, Thymoma and Thymic Carcinoma, Thyroid Cancer, Transitional Cell Cancer of the Renal Pelvis and Ureter, and Gestational Trophoblastic Tumor.

In one embodiment, the method of treatment of the present disclosure is useful for treating cancer, wherein the cancer is selected from the group consisting of Carcinoma of Unknown Primary Site, Cancer of Unknown Primary Site, Unusual Cancers of Childhood, Transitional Cell Cancer Of the Renal Pelvis and Ureter, Urethral Cancer, and Uterine Sarcoma. In one embodiment, the method of treatment of the present disclosure is useful for treating cancer, wherein the cancer is selected from the group consisting of Vaginal Cancer, and Vulvar Cancer. In one embodiment, the method of treatment of the present disclosure is useful for treating cancer, wherein the cancer is selected from the group consisting of Wilms Tumor, and Women's Cancers.

In some embodiments, treatment of cancer comprises prevention of tumor growth in a cancer subject. In some embodiments, treatment of cancer comprises prevention of formation of cancer metastases in a cancer subject. In some embodiments, treatment of cancer comprises targeted treatment of minimal residual disease in a cancer subject known to have the minimal residual disease in a cancer or a subject at risk for having minimal residual disease.

This might be indicated after treatment of the primary tumor by surgery and/or after chemotherapy (e.g. radiotherapy) has been initiated or determined to efficaceous. Disseminated tumor cells may be in their dormant state and often cannot be attacked by the chemotherapy (radiotherapy). A thus treated patient seemingly is in a healed state, which is also described as “minimal residual disease”. Nevertheless, the dormant tumor cells have a potential of forming metastases if they become metastasising cells due to a growth stimulus also after a longer dormant state.

As used herein, “minimal residual disease” denotes a small number of cancer cells that remain in in a subject during treatment, or after treatment when the subject is in remission (exhibiting no symptoms or signs of the disease). The methods described herein are preferably applied to any form of the diseases listed herein, including adult and childhood forms of these diseases.

In one embodiment, the method of treatment of the present disclosure is useful for treating an autoimmune disease. Autoimmune diseases include, but are not limited to alopecia areata, antiphospholipid, autoimmune hepatits, celiac disease, diabetes type 1, Graves' disease, Guillain-Barre syndrome, Hashimoto's disease, hemolytic anemia, idiopathic thrombocytopenic purpura, inflammator bowel disease, inflammatory myopathies, multiple sclerosis, primary biliary cirrhosis, psoriasis, rheumatoid arthritis, scleroderma, Sjögren's syndrome, systemic lupus erythematosus, and vitiligo.

In one embodiment, the method of treatment of the present disclosure is useful for treating autoimmune and inflammatory disorders of the peripheral nerve system such as amyotrophic lateral sclerosis (Lou Gehrig's disease), based on various causes such as metabolic disorders that include diabetes, B12 and folate vitamin deficiencies, chemotherapy medications and medicines used to treat HIV, poisons that cause peripheral nerve damage, cancers that develop peripheral neuropathies as well as paraneoplastic syndromes, alcohol abuse, chronic kidney disease, injuries that cause compression on nerves and other lesions, infections such as Lyme disease, Guillain Barre syndrome, connective tissue disease, rheumatoid arthritis, Sjogren's syndrome, systemic lupus erythematosus, certain inflammatory conditions such as sarcoidosis, coeliac disease, hereditary diseases such as charcot marie tooth syndrome, Friedreich's ataxia, and/or idiopathic where no specific cause is found but the inflammatory and/or autoimmune mechanisms are the cause of the onset.

In one embodiment, the method of treatment of the present disclosure is useful for treating autoimmune and inflammatory disorders with ocular manifestations. Such ocular manifestations include, but are not limited to, ocular cicatricial pemphigoid, Mooren's corneal ulcer, various forms of uveitis, rheumatoid arthritis, systemic lupus erythematosus, polyarteritis nodosa, relapsing polychondritis, Wegener's granulomatosis, scleroderma, Behcet's disease, Reiter's disease, inflammatory bowel disease (ulcerative colitis and Crohn's disease) and ankylosing spondylitis, retinitis pigmentosa, macular degeneration, keratoconjunctivitis sicca, scleritis, episcleritis, keratitis, peripheral corneal ulceration, and less common entities such as choroiditis, retinal vasculitis, episcleral nodules, retinal detachments, and/or macular edema.

In one embodiment, the method of treatment of the present disclosure is useful for treating acute allograft rejection in transplant patients. In one embodiment, the method of treatment of the present disclosure is useful for treating ischemic stroke. In one embodiment, the method of treatment of the present disclosure is useful for treating inflammatory diseases. Inflammatory diseases include, but are not limited to, arthritis, psoriasis, asthma, and colitis.

In some embodiments, the pharmaceutical composition in accordance with the present disclosure is administered in a method to a subject once daily. In some embodiments, a pharmaceutical composition in accordance with the present disclosure is administered to a subject according to an infrequent dosing regimen (e.g., administered once per week or less frequently). In some embodiments, a pharmaceutical composition in accordance with the present disclosure is administered to a subject according to a frequent dosing regimen (e.g., administered more than once per week). In some embodiments, the pharmaceutical composition in accordance with the present disclosure is administered to a subject once weekly. In some embodiments, the pharmaceutical composition in accordance with the present disclosure is administered to a subject once every four weeks. In some embodiments, the pharmaceutical composition in accordance with the present disclosure is administered to a subject twice a week. In some embodiments, the pharmaceutical composition in accordance with the present disclosure is administered to a subject once every two weeks. In some embodiments, the pharmaceutical composition in accordance with the present disclosure is administered to a subject once every three weeks. In some embodiments, the pharmaceutical composition in accordance with the present disclosure is administered to a subject in a repeated cycle of once weekly, once every two weeks, once every three weeks, once every four weeks or combinations thereof.

In one embodiment, the method of treatment comprises administering to a subject in need of such treatment: (i) a first therapeutic agent including a compound comprising ONC-206 or a pharmaceutically acceptable salt thereof in combination with (ii) a second therapeutic agent, wherein the first therapeutic agent and the second therapeutic agent are administered either simultaneously or sequentially; and further comprises assaying the expression of an endoplasmic reticulum (ER) stress response genes in a biological sample. In some embodiments the endoplasmic reticulum stress response gene is selected from the group that includes, but is not limited to, DRD2, CIpP, tyrosine hydroxylase, c-myc, n-myc, DR5, dopamine or its metabolites or catecholamines, C/EBP-Homologous Protein (CHOP), Activating Transcription Factor 3 (ATF3) and both CHOP and ATF3. The biological sample may be tumor, peripheral blood mononuclear cells, or skin biopsy. The biological sample may be obtained before, during, or after drug administration. In some embodiments, the method of treatment further comprises adjusting a dose of ONC-206 to achieve induction of about 50%, 75%, 100%, 125%, 150%, 175%, 200%, 225%, 250%, 275%, 300%, 325%, 350%, 375%, 400%, 425%, 450%, 475%, 500%, 525%, 550%, 575%, 600%, or greater than 600% of one or more ER stress gene(s). In some embodiments, the method of treatment further comprises adjusting a dose of ONC-206 to achieve induction of about 50% to about 100%, about 100% to about 150%, about 150% to about 200%, about 200% to about 250%, about 250% to about 300%, about 300% to about 350%, about 350% to about 400%, about 400% to about 450%, about 450% to about 500%, about 500% to about 550%, about 550% to about 600%, or greater than 600% of the ER stress gene. In some embodiments, the method of treatment further comprises adjusting a dose of ONC-206 to achieve induction of about 50% to about 100%, about 100% to about 200%, about 200% to about 300%, about 300% to about 400%, about 400% to about 500%, about 500% to about 600%, or greater than 600% of the ER stress gene.

In one embodiment, the method of treatment comprises administering to a subject in need of such treatment: (i) a first therapeutic agent including a compound comprising ONC-206 or a pharmaceutically acceptable salt thereof in combination with (ii) a second therapeutic agent, wherein the first therapeutic agent and the second therapeutic agent are administered either simultaneously or sequentially; and further comprises assaying the expression of a proteasomal activity in a biological sample. In some embodiments the proteasomal activity may be chymotrysin-like, trypsin-like, and/or caspase-like activity. In some embodiments the biological sample may be tumor, peripheral blood mononuclear cells, or skin cells. The biological sample may be obtained before, during, or after drug administration. In some embodiments, the method of treatment further comprises adjusting the dose to achieve inhibition of about 20%, about 25%, about 30%, about 35%, about 40%, about 45%, about 50%, about 55%, about 60%, about 65%, about 70%, about 75%, about 80%, about 85%, about 90%, about 95%, or about 100% of the proteasomal activity. In some embodiments, the method of treatment further comprises adjusting the dose to achieve inhibition of at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the proteasomal activity. In some embodiments, the method of treatment further comprises adjusting the dose to achieve inhibition of about 20% to about 30%, about 30% to about 40%, about 40% to about 50%, about 50% to about 60%, about 60% to about 70%, about 70% to about 80%, about 80% to about 90%, or greater than 90% of the proteasomal activity.

In an aspect, the present disclosure provides a method of treatment, which comprises administering to a subject in need of such treatment a combination of a first therapeutic agent including the following ONC-206 and a second therapeutic agent, the method comprising:
(i) administering to the subject the first therapeutic agent including ONC-206 or a pharmaceutically acceptable salt (e.g., a di-salt or tri-salt) thereof;
(ii) waiting until a predetermined waiting time has elapsed after the time of administration of the first therapeutic agent to the subject; and/or until adverse events are resolved or resolving; and
(iii) administering the second therapeutic agent to the subject, wherein the predetermined waiting time is chosen so as to obtain a delayed therapeutic effect of the first therapeutic agent without an increased risk of possible combined toxic effects of the first and second therapeutic agents. In some embodiments of the method of treatment, the predetermined waiting time is determined based on the clearance rate of ONC-206 or the pharmaceutically acceptable salt thereof. In some embodiments of the method of treatment, the predetermined waiting time is determined by a quantitative assessment of renal function and parameters of renal. In some embodiments of the method of treatment, the predetermined waiting time is determined by an assays for the determination of renal function, wherein the assay is selected from the group consisting of serum level of ONC-206 or the pharmaceutically acceptable salt thereof; ONC-206 or the pharmaceutically acceptable salt thereof clearance rate; 24-hour urinary clearance of ONC-206 or the pharmaceutically acceptable salt thereof or a metabolite thereof.

In one embodiment of the method of treatment, the predetermined waiting time substantially equals to the time required for systemic clearance of ONC-206 or a pharmaceutically acceptable salt thereof from the body of the subject. In one embodiment of the method of treatment, the predetermined waiting time substantially equals to the time required for renal clearance of ONC-206 or a pharmaceutically acceptable salt thereof from the body of the subject. In one embodiment of the method of treatment, the predetermined waiting time substantially equals to the time required for hepatic clearance of ONC-206 or a pharmaceutically acceptable salt thereof from the body of the subject. In one embodiment of the method of treatment, the predetermined waiting time substantially equals to the time required for total clearance of ONC-206 or a pharmaceutically acceptable salt thereof from the body of the subject. In one embodiment of the method of treatment, the predetermined waiting time is about 4 hours, 5 hours, 6 hours, 7 hours, 8 hours, 9 hours, 10 hours, 11, hours, or 12 hours. In other embodiments the waiting time is 1 day. In some embodiments, the wait time is until Cmax of ONC-206 has passed. In other embodiments, the waiting time is after most of the adverse events are resolved or are resolving. In one embodiment of the method of treatment, the predetermined waiting time is about 2 days. In one embodiment of the method of treatment, the predetermined waiting time is about 3 days. In one embodiment of the method of treatment, the predetermined waiting time is about 4 days. In one embodiment of the method of treatment, the predetermined waiting time is about 5 days. In one embodiment of the method of treatment, the predetermined waiting time is about 6 days. In one embodiment of the method of treatment, the predetermined waiting time is about 7 days. In one embodiment of the method of treatment, the predetermined waiting time is about 1-7 days. In one embodiment of the method of treatment, the predetermined waiting time is about 1-6 days. In one embodiment of the method of treatment, the predetermined waiting time is about 1-5 days. In one embodiment of the method of treatment, the predetermined waiting time is about 1-4 days. In one embodiment of the method of treatment, the predetermined waiting time is about 1-3 days. In one embodiment of the method of treatment, the predetermined waiting time is about 1 to 2 days. In some embodiments, the waiting time is up to 3 weeks. The preceeding are considered “therapeutic time priods.”

In one embodiment, the administration of ONC-206 is daily. In one embodiment, the administration of ONC-206 is every other day. In one embodiment, the administration of ONC-206 is every third day. In one embodiment, the administration of ONC-206 is every fourth day. In one embodiment, the administration of ONC-206 is every fifth day. In one embodiment, the administration of ONC-206 is every sixth day. In one embodiment, the administration of ONC-206 is weekly.

When the order of administration is reveresed, timing for the administration of ONC-206 can be after the Cmax of the first administered drug has passed. In some embodiments, administration of ONC-206 can be after most or substantially all of the first administered drug has been eliminated from the body or the toxicity effects for the first administered drug are resolved or are resolving.

In some embodiments, the method of treatment further comprises monitoring level of ONC-206, a pharmaceutically acceptable salt thereof, or a metabolite thereof in the subject using pharmacokinetic profiling. In some such embodiments, monitoring level of ONC-206, a pharmaceutically acceptable salt thereof, or a metabolite thereof in the subject using pharmacokinetic profiling comprises constructing a pharmacokinetic profile of ONC-206, a pharmaceutically acceptable salt thereof, or a metabolite thereof for the subject using concentrations of ONC-206, a pharmaceutically acceptable salt thereof, or a metabolite thereof in at least two samples obtained from the subject at time points suitable to construct a pharmacokinetic profile. In some embodiments of the method, which include monitoring level of ONC-206, a pharmaceutically acceptable salt thereof, or a metabolite thereof in the subject using pharmacokinetic profiling, at least two samples are collected from the subject at point-of-care or point of use by sampling or self-sampling on point-of-care devices or point of use devices or on matrices suitable for storage of the at least two samples prior to quantitation in a laboratory. In some embodiments of the method of treatment, each of the point-of-care devices or point of use devices is capable of quantitating ONC-206, a pharmaceutically acceptable salt thereof, or a metabolite. In some embodiments of the method, which include monitoring level of ONC-206, a pharmaceutically acceptable salt thereof, or a metabolite thereof in the subject, one or more samples are collected from the subject at point-of-care or point of use by biopsy device for analysis at the point-of-care or point of use devices or for storage prior to analysis by a laboratory. In some embodiments of the method, a biopsy is taken after a time interval of 3-8 hours following administration of ONC-206, a pharmaceutically acceptable salt thereof, or a metabolite thereof to the subject. In some embodiments of the method, a biopsy is taken after a time interval of 3-24 hours following administration of ONC-206, a pharmaceutically acceptable salt thereof, or a metabolite thereof to the subject. In some embodiments of the method, a biopsy is taken after a time interval of 8-24 hours following administration of ONC-206, a pharmaceutically acceptable salt thereof, or a metabolite thereof to the subject. In some embodiments of the method, a biopsy is taken after a time interval of 2 days following administration of ONC-206, a pharmaceutically acceptable salt thereof, or a metabolite thereof to the subject. In some embodiments of the method, a biopsy is taken after a time interval of 3 days following administration of ONC-206, a pharmaceutically acceptable salt thereof, or a metabolite thereof to the subject. In some embodiments of the method, a biopsy is taken after a time interval of 4 days following administration of ONC-206, a pharmaceutically acceptable salt thereof, or a metabolite thereof to the subject. In some embodiments of the method, a biopsy is taken after a time interval of 1-7 days following administration of ONC-206, a pharmaceutically acceptable salt thereof, or a metabolite thereof to the subject.

In some embodiments of the method of treatment, the pharmacokinetic profile includes pharmacokinetic parameters suitable for guiding dosing of ONC-206 or a pharmaceutically acceptable salt thereof for the subject being treated. In some embodiments of the method of treatment, maximum concentration of the first therapeutic agent in blood (whole blood, plasma, or serum) (“Cmax”) of the subject following its administration to the subject ranges from about 10 ng/mL to about 4000 ng/mL for a therapeutic time period, such as weekly or other than daily dose regimen. In some embodiments, Cmax is less than 4000 ng/mL and greater than 10 ng/mL for a therapeutic time period, such as weekly or other than daily dose regimen.

In some embodiments, maximum concentration of the first therapeutic agent in blood (whole blood, plasma, or serum) (“Cmax”) of the subject following its administration to the subject is a Cmax of from about 10 ng/mL to about 4000 ng/dl, including from about 10 ng/mL, from about 20 ng/mL, from about 30 ng/mL, from about 40 ng/mL, from about 50 ng/mL, from about 60 ng/mL, from about 70 ng/mL, from about 80 ng/mL, from about 90 ng/mL, from about 100 ng/mL, from about 110 ng/mL, from about 120 ng/mL, from about 130 ng/mL, from about 140 ng/mL, from about 150 ng/mL, from about 160 ng/mL, from about 170 ng/mL, from about 180 ng/mL, from about 190 ng/mL, from about 200 ng/mL, from about 210 ng/mL, from about 220 ng/mL, from about 230 ng/mL, from about 240 ng/mL, from about 250 ng/mL, from about 260 ng/mL, from about 270 ng/mL, from about 280 ng/mL, from about 290 ng/mL, from about 300 ng/mL, from about 310 ng/mL, from about 320 ng/mL, from about 330 ng/mL, from about 340 ng/mL, from about 350 ng/mL, from about 360 ng/mL, from about 370 ng/mL, from about 380 ng/mL, from about 390 ng/mL, from about 400 ng/mL, from about 410 ng/mL, from about 420 ng/mL, from about 430 ng/mL, from about 440 ng/mL, from about 450 ng/mL, from about 460 ng/mL, from about 470 ng/mL, from about 480 ng/mL, from about 490 ng/mL, from about 500 ng/mL, from about 510 ng/mL, from about 520 ng/mL, from about 530 ng/mL, from about 540 ng/mL, from about 550 ng/mL, from about 560 ng/mL, from about 570 ng/mL, from about 580 ng/mL, from about 590 ng/mL, from about 600 ng/mL, from about 610 ng/mL, from about 620 ng/mL, from about 630 ng/mL, from about 640 ng/mL, from about 650 ng/mL, from about 660 ng/mL, from about 670 ng/mL, from about 680 ng/mL, from about 690 ng/mL, from about 700 ng/mL, from about 710 ng/mL, from about 720 ng/mL, from about 730 ng/mL, from about 740 ng/mL, from about 750 ng/mL, from about 760 ng/mL, from about 770 ng/mL, from about 780 ng/mL, from about 790 ng/mL, from about 800 ng/mL, from about 810 ng/mL, from about 820 ng/mL, from about 830 ng/mL, from about 840 ng/mL, from about 850 ng/mL, from about 860 ng/mL, from about 870 ng/mL, from about 880 ng/mL, from about 890 ng/mL, from about 900 ng/mL, from about 910 ng/mL, from about 920 ng/mL, from about 930 ng/mL, from about 940 ng/mL, from about 950 ng/mL, from about 960 ng/mL, from about 970 ng/mL, from about 980 ng/mL, from about 990 ng/mL, from about 1000 ng/mL, from about 1010 ng/mL, from about 1020 ng/mL, from about 1030 ng/mL, from about 1040 ng/mL, from about 1050 ng/mL, from about 1060 ng/mL, from about 1070 ng/mL, from about 1080 ng/mL, from about 1090 ng/mL, from about 1100 ng/mL, from about 1110 ng/mL, from about 1120 ng/mL, from about 1130 ng/mL, from about 1140 ng/mL, from about 1150 ng/mL, from about 1160 ng/mL, from about 1170 ng/mL, from about 1180 ng/mL, from about 1190 ng/mL, from about 1200 ng/mL, from about 1210 ng/mL, from about 1220 ng/mL, from about 1230 ng/mL, from about 1240 ng/mL, from about 1250 ng/mL, from about 1260 ng/mL, from about 1270 ng/mL, from about 1280 ng/mL, from about 1290 ng/mL, from about 1300 ng/mL, from about 1310 ng/mL, from about 1320 ng/mL, from about 1330 ng/mL, from about 1340 ng/mL, from about 1350 ng/mL, from about 1360 ng/mL, from about 1370 ng/mL, from about 1380 ng/mL, from about 1390 ng/mL, from about 1400 ng/mL, from about 1410 ng/mL, from about 1420 ng/mL, from about 1430 ng/mL, from about 1440 ng/mL, from about 1450 ng/mL, from about 1460 ng/mL, from about 1470 ng/mL, from about 1480 ng/mL, from about 1490 ng/mL, from about 1500 ng/mL, from about 1510 ng/mL, from about 1520 ng/mL, from about 1530 ng/mL, from about 1540 ng/mL, from about 1550 ng/mL, from about 1560 ng/mL, from about 1570 ng/mL, from about 1580 ng/mL, from about 1590 ng/mL, from about 1600 ng/mL, from about 1610 ng/mL, from about 1620 ng/mL, from about 1630 ng/mL, from about 1640 ng/mL, from about 1650 ng/mL, from about 1660 ng/mL, from about 1670 ng/mL, from about 1680 ng/mL, from about 1690 ng/mL, from about 1700 ng/mL, from about 1710 ng/mL, from about 1720 ng/mL, from about 1730 ng/mL, from about 1740 ng/mL, from about 1750 ng/mL, from about 1760 ng/mL, from about 1770 ng/mL, from about 1780 ng/mL, from about 1790 ng/mL, from about 1800 ng/mL, from about 1810 ng/mL, from about 1820 ng/mL, from about 1830 ng/mL, from about 1840 ng/mL, from about 1850 ng/mL, from about 1860 ng/mL, from about 1870 ng/mL, from about 1880 ng/mL, from about 1890 ng/mL, from about 1900 ng/mL, from about 1910 ng/mL, from about 1920 ng/mL, from about 1930 ng/mL, from about 1940 ng/mL, from about 1950 ng/mL, from about 1960 ng/mL, from about 1970 ng/mL, from about 1980 ng/mL, from about 1990 ng/mL, from about 2000 ng/mL, from about 2010 ng/mL, from about 2020 ng/mL, from about 2030 ng/mL, from about 2040 ng/mL, from about 2050 ng/mL, from about 2060 ng/mL, from about 2070 ng/mL, from about 2080 ng/mL, from about 2090 ng/mL, from about 2100 ng/mL, from about 2110 ng/mL, from about 2120 ng/mL, from about 2130 ng/mL, from about 2140 ng/mL, from about 2150 ng/mL, from about 2160 ng/mL, from about 2170 ng/mL, from about 2180 ng/mL, from about 2190 ng/mL, from about 2200 ng/mL, from about 2210 ng/mL, from about 2220 ng/mL, from about 2230 ng/mL, from about 2240 ng/mL, from about 2250 ng/mL, from about 2260 ng/mL, from about 2270 ng/mL, from about 2280 ng/mL, from about 2290 ng/mL, from about 2300 ng/mL, from about 2310 ng/mL, from about 2320 ng/mL, from about 2330 ng/mL, from about 2340 ng/mL, from about 2350 ng/mL, from about 2360 ng/mL, from about 2370 ng/mL, from about 2380 ng/mL, from about 2390 ng/mL, from about 2400 ng/mL, from about 2410 ng/mL, from about 2420 ng/mL, from about 2430 ng/mL, from about 2440 ng/mL, from about 2450 ng/mL, from about 2460 ng/mL, from about 2470 ng/mL, from about 2480 ng/mL, from about 2490 ng/mL, from about 2500 ng/mL, from about 2510 ng/mL, from about 2520 ng/mL, from about 2530 ng/mL, from about 2540 ng/mL, from about 2550 ng/mL, from about 2560 ng/mL, from about 2570 ng/mL, from about 2580 ng/mL, from about 2590 ng/mL, from about 2600 ng/mL, from about 2610 ng/mL, from about 2620 ng/mL, from about 2630 ng/mL, from about 2640 ng/mL, from about 2650 ng/mL, from about 2660 ng/mL, from about 2670 ng/mL, from about 2680 ng/mL, from about 2690 ng/mL, from about 2700 ng/mL, from about 2710 ng/mL, from about 2720 ng/mL, from about 2730 ng/mL, from about 2740 ng/mL, from about 2750 ng/mL, from about 2760 ng/mL, from about 2770 ng/mL, from about 2780 ng/mL, from about 2790 ng/mL, from about 2800 ng/mL, from about 2810 ng/mL, from about 2820 ng/mL, from about 2830 ng/mL, from about 2840 ng/mL, from about 2850 ng/mL, from about 2860 ng/mL, from about 2870 ng/mL, from about 2880 ng/mL, from about 2890 ng/mL, from about 2900 ng/mL, from about 2910 ng/mL, from about 2920 ng/mL, from about 2930 ng/mL, from about 2940 ng/mL, from about 2950 ng/mL, from about 2960 ng/mL, from about 2970 ng/mL, from about 2980 ng/mL, from about 2990 ng/mL, from about 3000 ng/mL, from about 3010 ng/mL, from about 3020 ng/mL, from about 3030 ng/mL, from about 3040 ng/mL, from about 3050 ng/mL, from about 3060 ng/mL, from about 3070 ng/mL, from about 3080 ng/mL, from about 3090 ng/mL, from about 3100 ng/mL, from about 3110 ng/mL, from about 3120 ng/mL, from about 3130 ng/mL, from about 3140 ng/mL, from about 3150 ng/mL, from about 3160 ng/mL, from about 3170 ng/mL, from about 3180 ng/mL, from about 3190 ng/mL, from about 3200 ng/mL, from about 3210 ng/mL, from about 3220 ng/mL, from about 3230 ng/mL, from about 3240 ng/mL, from about 3250 ng/mL, from about 3260 ng/mL, from about 3270 ng/mL, from about 3280 ng/mL, from about 3290 ng/mL, from about 3300 ng/mL, from about 3310 ng/mL, from about 3320 ng/mL, from about 3330 ng/mL, from about 3340 ng/mL, from about 3350 ng/mL, from about 3360 ng/mL, from about 3370 ng/mL, from about 3380 ng/mL, from about 3390 ng/mL, from about 3400 ng/mL, from about 3410 ng/mL, from about 3420 ng/mL, from about 3430 ng/mL, from about 3440 ng/mL, from about 3450 ng/mL, from about 3460 ng/mL, from about 3470 ng/mL, from about 3480 ng/mL, from about 3490 ng/mL, from about 3500 ng/mL, from about 3510 ng/mL, from about 3520 ng/mL, from about 3530 ng/mL, from about 3540 ng/mL, from about 3550 ng/mL, from about 3560 ng/mL, from about 3570 ng/mL, from about 3580 ng/mL, from about 3590 ng/mL, from about 3600 ng/mL, from about 3610 ng/mL, from about 3620 ng/mL, from about 3630 ng/mL, from about 3640 ng/mL, from about 3650 ng/mL, from about 3660 ng/mL, from about 3670 ng/mL, from about 3680 ng/mL, from about 3690 ng/mL, from about 3700 ng/mL, from about 3710 ng/mL, from about 3720 ng/mL, from about 3730 ng/mL, from about 3740 ng/mL, from about 3750 ng/mL, from about 3760 ng/mL, from about 3770 ng/mL, from about 3780 ng/mL, from about 3790 ng/mL, from about 3800 ng/mL, from about 3810 ng/mL, from about 3820 ng/mL, from about 3830 ng/mL, from about 3840 ng/mL, from about 3850 ng/mL, from about 3860 ng/mL, from about 3870 ng/mL, from about 3880 ng/mL, from about 3890 ng/mL, from about 3900 ng/mL, from about 3910 ng/mL, from about 3920 ng/mL, from about 3930 ng/mL, from about 3940 ng/mL, from about 3950 ng/mL, from about 3960 ng/mL, from about 3970 ng/mL, from about 3980 ng/mL, from about 3990 ng/mL, and from about 4000 ng/mL

In some embodiments of the method the total drug exposure over time, measured as the area under the curve (“AUC”) of a plot of the concentration of the drug in blood (whole blood, plasma, or serum) of the subject following administration of the drug against time after administration of the drug ranges from about 10 ng hr/ml to about 20000 ng hr/ml. In some embodiments, AUC is less than 20000 ng hr/ml, 19000 ng hr/ml, 18000 ng hr/ml, 17000 ng hr/ml, 16000 ng hr/ml, 15000 ng hr/ml, 14000 ng hr/ml, 13000 ng hr/ml, 12000 ng hr/ml, 11000 ng hr/ml, 10000 ng hr/ml, 9000 ng hr/ml, 8000 ng hr/ml, 7000 ng hr/ml, 6000 ng hr/ml, 5000 ng hr/ml, 4000 ng hr/ml, 3000 ng hr/ml, 2000 ng hr/ml, 1000 ng hr/ml, 900 ng hr/ml, 800 ng hr/ml, 700 ng hr/ml, 600 ng hr/ml, 500 ng hr/ml, 400 ng hr/ml, 300 ng hr/ml, 200 ng hr/ml, 100 ng hr/ml, 90 ng hr/ml, 80 ng hr/ml, 70 ng hr/ml, 60 ng hr/ml, 50 ng hr/ml, 40 ng hr/ml, 30 ng hr/ml, 20 ng hr/ml, and 10 ng hr/ml.

In another aspect, the present disclosure provides a method of treatment, or use of the composition to treat a disease state, which comprises administering to a subject in need of such treatment a combination of a first therapeutic agent and a second therapeutic agent, the method comprising:
(i) administering to the subject the first therapeutic agent including ONC-206 or a pharmaceutically acceptable salt thereof;
(ii) monitoring level of ONC-206 or a pharmaceutically acceptable salt thereof or a metabolite thereof in the subject using pharmacokinetic profiling; and
(iii) administering the second therapeutic agent conditional on the level of the first therapeutic agent in the subject. In some embodiments of the method, the monitoring step includes constructing a pharmacokinetic profile of ONC-206 or a pharmaceutically acceptable salt thereof or a metabolite thereof for the subject using concentrations of ONC-206 or a pharmaceutically acceptable salt thereof or a metabolite thereof in at least two samples obtained from the subject at time points suitable to construct a pharmacokinetic profile. In some embodiments of the method, the at least two samples are collected at point-of-care or point of use by sampling or self-sampling on point-of-care devices or point of use devices or on matrices suitable for storage of the at least two samples prior to quantitation of ONC-206 or a pharmaceutically acceptable salt thereof or a metabolite by a laboratory. In some embodiments of the method, each point-of-care devices or point of use devices is capable of quantitating ONC-206 or a pharmaceutically acceptable salt thereof or a metabolite. In some embodiments of the method, the pharmacokinetic profile includes pharmacokinetic parameters suitable for guiding dosing of ONC-206 or a pharmaceutically acceptable salt thereof for the subject. In some embodiments of the method, the at least two samples include from 2-12 samples. In some embodiments of the method, the at least two samples are collected over a time period of up to 8 hours, up to 24 hours, up to 48 hours, or up to 72 hours. In some embodiments of the method, the pharmacokinetic parameters include at least one parameter selected from the group consisting of AUC, AUCinf, Tmax, Cmax, time above threshold, steady state concentration, absorption rate, clearance rate, distribution rate, terminal T-½ or parameters drawn from noncompartmental pharmacokinetic (PK) or compartmental PK analysis, including physiological model-based compartmental PK analysis. In some embodiments of the method, the method of treatment further comprises generating a report including the pharmacokinetic profile of the subject. In some embodiments of the method, the report includes a recommendation regarding dosing based on the pharmacokinetic profile of the subject. In some embodiments of the method, a reduction in dosage of ONC-206 or a pharmaceutically acceptable salt thereof is indicated to reduce risk of toxicity based on one or more pharmacokinetic parameters. In some embodiments of the method, the reduction in dosage of ONC-206 or a pharmaceutically acceptable salt thereof is indicated based on time above threshold, wherein the threshold is the drug concentration above which toxicity occurs, or one or more of AUC, AUCinf, mean residence time (MRT), exponentials defining the pharmacokinetic profile, volume of distribution at steady state (Vss), volume of distribution during the terminal phase (Vz) or combination of a group of pharmacokinetic variable to adequately describe the pharmacokinetic profile. In some embodiments of the method, a dose adjustment of ONC-206 or a pharmaceutically acceptable salt thereof is indicated to increase efficacy based on one or more pharmacokinetic parameters. In some embodiments of the method, an increase in dosage of ONC-206 or a pharmaceutically acceptable salt thereof is indicated based on one or more of AUC, AUCinf, MRT, exponentials defining the pharmacokinetic profile, steady state volume (Vss) of distribution, volume of distribution during the terminal phase (Vz) or combination of a group of pharmacokinetic variables to adequately describe the pharmacokinetic profile. In some embodiments of the method, the dose of ONC-206 or a pharmaceutically acceptable salt thereof is adjusted to within 5% to 25% of a desired target value. In some embodiments of the method, each of the at least two samples is applied to the point-of-care device or the point of use device for determining the concentration of the ONC-206 or a pharmaceutically acceptable salt thereof or a metabolite thereof, wherein the point-of-care device or the point of use device comprises a lateral flow strip having a construction and composition such that an application of one or more of the at least two samples to the lateral flow strip causes a fraction of the drug in the sample to bind to with a component of the lateral flow strip such that a detectable signal proportional to the concentration of the drug in the applied sample is produced. In some embodiments of the method, the at least two samples are applied to matrices suitable for storage of the at least two samples prior to quantitation by a laboratory. In some embodiments of the method, the at least two samples are stored as dried blood spots. In some embodiments of the method, drug concentrations are measured by ELISA, LC MS MS, LC UV or LCMS. In some embodiments of the method, the pharmacokinetic parameters include at least one of steady state concentration, absorption, and terminal T½. In some embodiments of the method, at least one of the at least two samples is whole blood.

### V. MULTIMODAL THERAPEUTIC METHODS

In one aspect, the present disclosure is directed to multimodal therapeutic methods in which administration of ONC-206 or a pharmaceutically acceptable salt thereof to a subject in need of such treatment is supplemented by administration of other therapeutic modalities. In one embodiment, the multimodal therapeutic method of the present disclosure comprises administering to a subject a pharmaceutical composition comprising the ONC-206 or a pharmaceutically acceptable salt thereof in conjunction with radiation therapy or after radiation is determined to not have been efficacious. In one embodiment, the multimodal therapeutic method of the present disclosure comprises administering to a subject a pharmaceutical composition comprising ONC-206 or a pharmaceutically acceptable salt thereof in conjunction with radiation therapy, wherein the pharmaceutical composition comprising ONC-206 or a pharmaceutically acceptable salt thereof and the radiation therapy are administered concurrently or sequentially in any order. In one embodiment, the multimodal therapeutic method comprises administering to a subject a pharmaceutical composition comprising ONC-206 or a pharmaceutically acceptable salt thereof in conjunction with radiation therapy in a sequential arrangement. In one embodiment, the multimodal therapeutic method comprises administering to a subject in need of such treatment a pharmaceutical composition comprising ONC-206 or a pharmaceutically acceptable salt thereof concurrently with radiation therapy. In one embodiment, the multimodal therapeutic method of the present disclosure is used for the treatment of cancer. In one embodiment, the multimodal therapeutic method includes administering to a cancer subject in need of such treatment a pharmaceutical composition comprising ONC-206 or a pharmaceutically acceptable salt thereof and irradiating cancer cells with a radiation beam. In one embodiment, the multimodal therapeutic method uses the technique of conformal radiotherapy (CRT) to deliver a dose volume histogram (DVH) prescribed to a cancer subject. In one embodiment, the multimodal therapeutic method uses the technique of intensity modulated radiation therapy (IMRT) to deliver radiation to cancer cells. In one embodiment, the multimodal therapeutic method uses a techniques compensates for motion of tumors in the subject during treatment (e.g., where doses of radiation must be administered to a thoracic tumor which moves as the patient breathes). In one embodiment, the multimodal therapeutic method use Four Dimensional Computed Tomography (4D CT) scanning techniques to adjust the delivered radiation field to compensate for tumor motion over the breathing cycle.

Any suitable type of radiation, including gamma radiation which is given fractionated, IMRT (intensity modulated radiation therapy), gamma knife, proton therapy and brachytherapy can be used with the multimodal therapeutic method of the present disclosure. Radiation therapy and ONC-206 or a pharmaceutically acceptable salt thereof can be used to treat brain tumors such as glioblastoma or disease that has metastasized to the brain from lung cancer, neuroendocrine tumors, or endometrial cancers. The multimodal therapeutic method of the present disclosure can be used to treat lung cancer, pancreatic cancer, rectal cancer, breast cancer, sarcoma, prostate cancer, gynecological malignancies, and lymphoma. The gamma knife is used frequently to treat brain metastases. In one embodiment, the multimodal therapeutic method of the present disclosure includes use of proton therapy to treat cancer, including brain tumors, prostate cancer and any tumor proximate vital organs where it is very important to minimize toxicity to nearby normal tissue.

In one embodiment, the multimodal therapeutic method of the present disclosure eliminates minimal residual disease without adding to any toxicity resulting from treatment ONC-206 or a pharmaceutically acceptable salt thereof. In one embodiment, the multimodal therapeutic method of the present disclosure improves prognosis and/or reduces adverse side-effects associated with a disease state or condition in a subject undergoing treatment.

### VI. DERIVATIVES AND ANALOGS OF AND SALTS OF ONC-206 AND RELATED COMPOUNDS

In one aspect, the present disclosure provides analogs and related salts of ONC-206 and processes of making the same. Persons skilled in the art will understand that the same general principles and concepts described above in conjunction with ONC-206 and salts thereof, including principles and concepts related to methods and pharmaceutical compositions, apply with equal force to derivatives and analogs of and salts of ONC-206 and salts thereof.

In one embodiment, the compounds related to ONC-206 have the structure of compound (10):

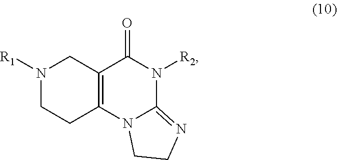

wherein R₁ and R₂ independently represent hydrogen, alkyl, cycloalkyl, cycloalkylalkyl, carboxyl, haloalkyl, alkenyl, cycloalkenyl, alkynyl, aryl, aralkyl, hydroxyalkyl, alkoxy, aryloxy, alkoxyalkyl, alkoxycarbonyl, aralkoxy, aralkylthio, alkanoyl, mercapto, alkylthio, arylthio, alkylsulfinyl, arylsulfinyl, alkylsulfonyl, arylsulfonyl, heteroaryl, acyl, and heterocycle radicals. In another embodiment, the compounds related to ONC-206 have the structure of compound (10), wherein R₁ and R₂ are independently selected from the group consisting of H, C₁₋₄alkyl, C₁₋₄alkylphenyl, C₁₋₄alkylphenylketone, C₁₋₄benzyl-piperazine, and C₁₋₄alkylthienyl wherein C₁₋₄alkyl, C₁₋₄alkylphenyl, C₁₋₄alkylphenylketone, and C₁₋₄benzyl-piperazine are optionally substituted with C₁₋₄alkyl, hydroxyl, or halo. In still another embodiment, the compounds related to ONC-206 have the structure of compound (10), wherein R₁ and R₂ are independently selected from the group consisting of H, CH₃, CH₂Ph, CH₂-((2-Cl)-Ph), CH₂-(2-thienyl), CH₂CH₂Ph, CH₂CH₂(4-N-benzyl-piperazine), CH₂-(2,4-di F-Ph), CH₂-((2-CH₃)-Ph), CH₂CHOHPh, and (CH₂)₃CO-4F-Ph. In some embodiments, when R₁ represents CH₂Ph, R₂ does not represent CH₂-((2-CH₃)-Ph.

As illustrated in Schemes 1 and 2, compound (10) can be synthesized starting either with methyl 1-R₁-4-oxo-3-piperidinecarboxylate (6) or by reacting compound (12) with compound (6).

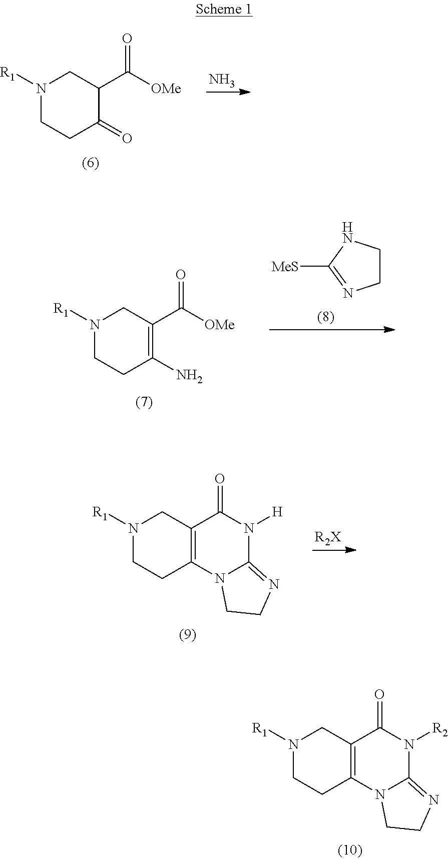

Scheme 1 illustrates the synthesis of compound (10) starting from compound (6). In one embodiment, as illustrated in Scheme 3, compound (6) was converted into 4-amino-3-pyridinecarboxylic acid ester methyl ester (7) (or methyl 4-amino-1-R₁-1,2,5,6-tetrahydro-3-pyridinecarboxylate) by a reaction with ammonia. In one embodiment, compound (7) (or 4-amino-3-pyridinecarboxylic acid ester methyl ester (7)) was treated with 2-(Methylsulfanyl)-4,5-dihydro-1H-imidazole (8) to make compound (9), which when alkylated R₂X, wherein R₂ is as defined above and X is a halogen or an equivalent leaving group, produced compound (10) with different values for the R₂ substituent.

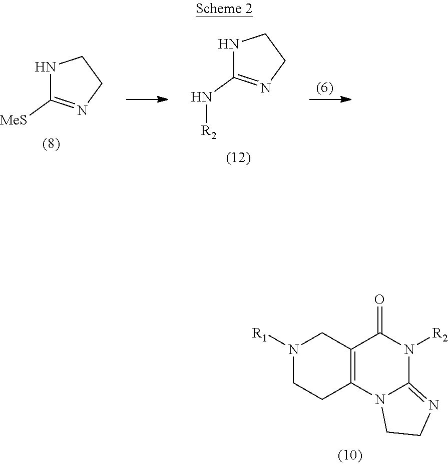

Scheme 2 illustrates the synthesis of compound (10) starting from compound (6) and compound (12). In one embodiment, as illustrated in Scheme 4, compound (12) is prepared from compound (8). In one embodiment, compound (12) was treated with compound (6) to produce compound (10) with different values for the R₂ substituent.

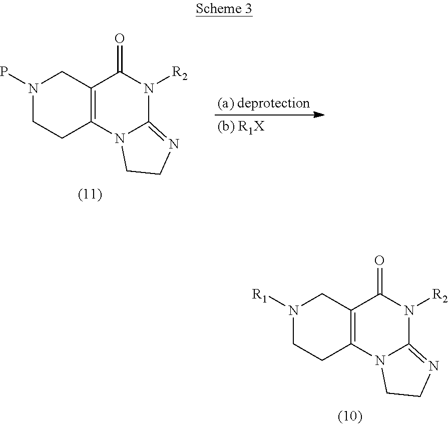

Scheme 3 illustrates the synthesis of compound (10) starting from compound (11). In one embodiment, as illustrated in Scheme 5, compound (11), having a nitrogen protecting group (P) at the N atom at ring position 7, was first deprotected and then akylated with R₁X, wherein R₁ is as defined above and X is a halogen or an equivalent leaving group, to produce compound (10) with different values for the R₁ substituent. In some embodiments compound (10) can be prepared as a salt, for example a 2TFA salt or 2HCl salt. In some embodiments, compound (10) can be prepared as a 2HCl salt.

### EXAMPLES OF COMPOUND (10)

| | ONC | | |
|---|---|---|---|
| No. | Number | R | R₂ |
| 1 | ONC201 | CH₂Ph | CH₂—((2—CH₃)—Ph) |
| 13 | | CH₂Ph | CH₃ |
| 14 | ONC202 | CH₂Ph | CH₂—((2—Cl)—Ph) |
| 15 | ONC203 | CH₂Ph | CH₂—(2-thienyl) |
| 16 | ONC204 | CH₂Ph | CH₂CH₂Ph |
| 17 | ONC205 | CH₂Ph | CH₂CH₂(4-N-benzyl- |
| | | | piperazine) |
| 18 | ONC206 | CH₂Ph | CH₂—(2,4-di F—Ph) |
| 19 | ONC207 | H | CH₂—((2—CH₃)—Ph) |
| 20 | ONC208 | CH₃ | CH₂—((2—CH₃)—Ph) |
| 21 | ONC209 | CH₂CH₂Ph | CH₂—((2—CH₃)—Ph) |
| 22 | | CH₂CH₂-(4-N-benzyl- | CH₂—((2—CH₃)—Ph) |
| | | piperizine) | |
| 23 | | CH₂CHOHPh | CH₂—((2—CH₃)—Ph) |
| 24 | | (CH₂)₃CO—4F—Ph | CH₂—((2—CH₃)—Ph) |
| 32 | ONC210 | CH₂CH₂NHCOOC(CH₃)₃ | CH₂—((2—CH₃)—Ph) |
| 33 | ONC211 | CH₂CH₂CH₂NH₂ | CH₂—((2—CH₃)—Ph) |

### VII. EXAMPLES

It should be understood that the description and specific examples provided below are intended for purposes of illustration only and are not intended to limit the scope of the present disclosure. The following examples are intended to illustrate the embodiments disclosed and are not to be construed as being limitations thereto. Additional compounds, other than those described below, may be prepared using the following reaction schemes described above or appropriate variations or modifications thereof.

### Example 1. Synthesis of 2-Chlorobenzylamino-2-imidazoline hydriodide

To a stirred solution of 2-methylthio-2-imidazoline hydriodide (244 mg, 1.00 mMol) in dry dioxane (2.0 mL) was added 2-chlorobenzylamine (141 mg, 1.0 mMol). The reaction mixture was stirred for 90 min at 70 C. under an atmosphere of argon. The solution was cooled to room temperature, filtered on a sintered funnel, washed with cold dioxane (2 mL) and dried under vacuum. The white solid compound 4·HI (R₂=2-chlorobenzyl) was obtained (242 mg, 72%) and used without further purification.

### Example 2. Synthesis of 2-Chlorobenzylamino-2-imidazoline

To a stirred solution of 2-chlorobenzylamino-2-imidazoline hydriodide (242 mg, 0.72 mMol) in water (3 mL), was added 1.0 N sodium hydroxide (2 mL) at 7° C. The reaction mixture was stirred for 30 min at 7° C. under argon. After that methylene chloride (5 mL) was added and the mixture stirred for another 5 min. The reaction mixture was extracted with methylene chloride (2×2.5 mL), The organic layer was dried over anhydrous Na₂SO₄, filtered and evaporated. The resulting free base (150 mg, 100%) was obtained as a viscous liquid and was used for the next reaction without any further purification. MS(ESI) 210(M+H).

### Example 3. Synthesis of Methyl-1-benzyl 4-oxo-3-piperidine carboxylate (Compound (6))

To a stirred methyl-1-benzyl 4-oxo-3-piperidine carboxylate hydrochloride (5.7 g, 20 mMol) in ethyl acetate (50 mL), was added triethylamine (6 mL) at 7° C. The reaction mixture was stirred for 30 min at 7° C. under atmosphere of argon. The reaction mixture was extracted with ethyl acetate (2×50 mL) washed with water (50 mL). The organic layer was dried over anhydrous Na₂SO₄, filtered and evaporated. The resulting free base residue (5, R₁=benzyl) as a viscous oil was used in the next reaction without any further purification MS(ESI) 248(M+H)

### Example 4. Synthesis of ONC202 (Compound (14))

To a solution of 2-chlorobenzylamino-2-imidazoline (150 mg, 0.72 mMol), methyl 1-benzyl 4-oxo-3-piperidine carboxylate (5, R₁=benzyl) (195 mg, 0.79 mMol) in 1-butanol (2 mL) was added PPTS (10 mg) and the mixture was stirred at room temperature for 48 h. After that the reaction mixture was refluxed at 125° C. to 130° C. for 2 h. The solvents were removed under vacuum, extracted with ethyl acetate (10 mL), washed with saturated sodium bicarbonate solution (2×10 mL) and water (10 mL). The organic layer was dried over anhydrous Na₂SO₄, filtered and evaporated. The crude free base was purified by RP HPLC (10%-40% acetonitrile/water) to give ONC902 TFA salt as a white solid (228 mg, 50% yield) MS(ESI) 407 (M+H).

The same process was used starting with different benzylamines to prepare various analogs, e.g., ONC203, 204, 205, and 206.

### Description of Manufacturing Process and Process Controls

The chemical synthesis of ONC206 and its conversion to the dihydrochloride (ONC206·2HCl) was accomplished following the process shown in Scheme 1.

In this route, the piperidone ester (Cpd 2·HCl) was converted to the Cpd 2 (free base) and condensed with the benzyl imidazoline (Cpd 10) to yield the ONC206 free base. The ONC206 free base was converted to the dihydrochloride and the final product ONC206·2HCl was crystallized from ethanol. The step-by-step procedure is summarized in Table 3, the major equipment is listed in Table 4, followed by a description of the process.

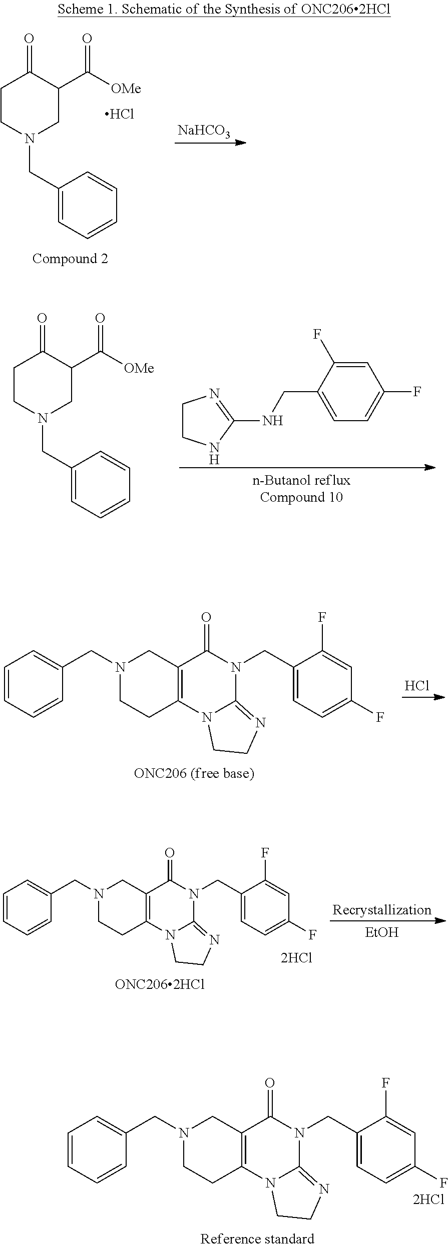

### Step, Procedure Description

1, To a stirred NaHCO₃ aqueous solution, charge compound 2·HCl in portions.
2, Add n-butanol to the above mixture. Stir for 30 min.
3, Separate the n-butanol phase and dry with MgSO₄.
4, To a three-neck flask equipped with a mechanical stirrer, a Dean-Stark trap, a condenser, a thermocouple and N₂ inlet, charge compound 10, (Pyridinium p-toluenesulfonate) PPTS and the above compound 2 solution in n-butanol.
5, Heat the mixture to reflux.
6, Stop the reaction when the peak area of the product by HPLC remains constant over time.
7, Wash the mixture with water.
8, Dilute the organic phase with Methyl tert-butyl ether (MTBE).
9, Wash the combined organic phase with water.
10, Transfer the organic phase to a cleaned flask.
11, Dilute 4N HCl in dioxane to 2N HCl solution with MTBE.
12, Add 2H HCl solution to the organic phase, until no more solid precipitates out on the surface at the addition of HCl.
13, Heat the mixture at reflux for 2 hours. Separate the water by a Dean-Stark trap.
14, After cooling to room temperature, filter off the solid.
15, Wash the solid with a 1:2 mixture of n-Butanol and MTBE.
16, Dry the solid under vacuum.
17, Charge the dry solid to a flask, followed by ethanol.
18, Heat the mixture to until all the solid dissolves and filter.
19, Cool the mixture and filter off the solid.
20, Wash the solid with ethanol.
21, Dry the solid under vacuum until it reaches a constant weight.

### Tablulated Summary of Procedural Steps for Synthesis of ONC206·2HCl

Equipment Name

22 L Reactor
50 L Reactor
Table Top Ceramic Filter
Drying Trays
Vacuum Drying Oven

### Equipment Involved in Synthesis of ONC206·2HCl.

### Brief Description of the Manufacturing Process

To a stirred NaHCO₃ aqueous solution, charge To a stirred solution NaHCO₃ (859 g; 10.2 mol) in 8400 mL of distilled water in a 50 L reactor, was added the benzylpiperidinone ester HCl salt (compound 2 hydrochloride (2743 g, 9.667 mol, 1.70 equiv)) in portions. n-Butanol (8400 mL) was then added to the mixture. The mixture was stirred for 30 min and transferred to a separatory funnel. The organic phase was separated and dried by stirring over 1000 g of for 2 hr. The MgSO₄ was filtered off and washed with 1000 mL of n-butanol and transferred to a 22 L reactor equipped with mechanical stirring, N2 inlet, a thermocouple, a condenser and a Dean-Stark trap. The imidazoline compound 10 (1200 g, 0.5685 mol, 1.00 equiv) and PPTS (71.5 g, 0.237 mol, 4.2 mol %) were added to the reactor and stirred overnight. The mixture was heated to reflux and maintained at reflux until the peak area of the product by HPLC remained constant over time. This condition was met and the mixture allowed to cool to room temperature after 4 h.

The mixture was transferred to a 50 L reactor with a bottom valve and was washed with 8400 mL of water. The organic phase was diluted with MTBE (16800 mL) and washed with water (2×8400 mL) and transferred to a 50 L reactor equipped with mechanical stirring, N2 inlet, a thermocouple, a condenser and a Dean-Stark trap. HCl (2N in dioxane, 3128 mL) was diluted with an equal volume of MBTE (3128 mL) and the solution of 1 N HCl in dioxane-MTBE was added until no more solid precipitated out on the surface at the addition of HCl (5600 mL). The mixture was heated at reflux at 60-65° C. for 2 hr with the water separating in the Dean-Stark trap. After cooling to room temperature, the solid was filtered off using a table top ceramic filter and washed with n-butanol:MTBE (1:2, 7200 mL). The solid was dried in drying trays in a vacuum oven at 55° C. for 4 hr, then room temperature for 60 h, to afford 2305 g (84% yield) of the crude product as a yellow solid.

To a 22 L reactor equipped with mechanical stirring, nitrogen inlet, a thermocouple and a condenser, the crude solid (2300 g) was added, followed by ethanol (11500 mL). The mixture was heated until it completely dissolved (67° C.) and the hot solution filtered. The solution was cooled slowly to room temperature overnight with stirring, then to 15° C. for 1 hour. The solid was filtered off, washed with ethanol (3×1400 mL)-transferred to drying trays, and dried in the vacuum oven at 75° C. for until a constant weight was reached. 1492 g of ONC206·2HCl was obtained as an off-white solid in a yield of 65%.

### Example 5. Synthesis of ONC206

To a stirred 800 mL saturated NaHCO₃ in a 2 L round bottom flask, compound (3) (239.7 g, 0.845 mol, 1.6 equiv) was added in portions. n-Butanol (500 mL) was added to the resulting mixture and the mixture was stirred for 30 min and then transferred to a separating funnel. The organic phase, containing compound (4), was separated and transferred to a 2 L three-neck round bottom flask equipped with mechanical stirring, N₂ inlet, a thermocouple, a condenser and a Dean-Stark trap. Compound (5) (100 g, 0.528 mol, 1 equiv) and pyridinium p-toluenesulfonate (PPTS) (6.63 gm 0.026 mol, 5 mol %) were added to the contents of the flask. The resulting mixture was heated to reflux for 6 hours. Water in the reaction mixture was separated into the Dean-Stark trap as necessary. Refluxing temperature increased from 93° C. to 118° C. Reaction progress was monitored by HPLC. When the peak area of ONC-206 on HPLC remained constant with the reaction time, the reaction was stopped.

### Example 6. Synthesis of Di-Salt of ONC206

Without isolation of the ONC-206, the reaction mixture from EXAMPLE 8 was washed with 500 mL of water and diluted with methyl tert-butyl ether (MTBE) (800 mL). The organic phase was washed with water (500 mL×2) and transferred to a 3 L three-neck round bottom flask equipped with mechanical stirring, N2 inlet, a thermocouple, a condenser and a Dean-Stark trap. While agitating the reaction mixture, 1 N HCl in dioxane-MTBE solution was added dropwise (4 N HCl in dioxane: 300 mL, 1.2 mol, 2.27 equiv; MTBE: 1200 mL) until no more solid precipitated out of the reaction mixture upon addition of HCl. The reaction mixture was heated to reflux at 60-65° C. for 2 hours. Water was separated into the Dean-Stark trap as necessary. Upon cooling to room temperature, the solid precipitate was filtered through a sintered glass funnel and washed with n-butanol-MTBE (1: 2, 600 mL) and MTBE (600 mL) respectively. The solid was dried in the vacuum oven at 65° C. overnight (16 hours) to afford 200 g yellow solid.

To a 2 L three-neck round bottom flask equipped with mechanical stirring, N2 inlet, a thermocouple and a condenser, the above solid (200 g) was added, followed by ethanol (1000 mL). The mixture was heated to reflux at 78° C. for 2 hours. Upon cooling to room temperature, the solid was filtered through a sintered glass funnel and washed with ethanol (200 mL×3). The wet solid was dried in the vacuum oven at 85° C. for 3 days until the residual solvent met specification. 120 g of compound (2) was obtained as a white solid in a yield of 49%, with HPLC purity 99.7%.

### Biological Examples

ONC-206 is orally bioavailable, penetrates the blood-brain barrier, and exhibits anti-cancer efficacy without toxicity in several preclinical cancer models with pronounced efficacy in myc-overexpressing CNS tumors.

A first-in-human, open label, dose escalation, and food effect Phase I study of oral ONC206 (NCT04541082) is performed. Criteria include that patients must be 18 years or older and diagnosed with a recurrent, primary CNS neoplasm. Eligible diseases include recurrent glioblastoma, WHO Grade 2 and 3 infiltrating glial neoplasms, DMG H3K27M, ependymoma, medulloblastoma, malignant meningiomas, and other rare primary CNS neoplasms. Dose escalation, initially with weekly dosing, will follow a standard 3+3 design. After the maximum tolerated dose (“MTD”) is established, a food effect cohort will enroll with a balanced, single-dose, two-arm, two-period, crossover design. The primary endpoint is to determine a dose-limiting toxicity (“DLT”), if applicable, during the first 28-day cycle. Secondary endpoints will include objective response rate by RANO criteria, overall and progression-free survival, and disease control rate. Exploratory biomarker analyses based on preclinical correlations with efficacy will include DRD2, DRD2 dimer, CIpP, DRD5, c-myc, and n-myc expression.

ONC206 is a small molecule that antagonizes the G protein-coupled receptors (GPCRs) dopamine receptor D2 (DRD2) and D3 (DRD3). Downstream of target engagement, ONC206 causes activation of ISR, inactivation of pro-survival Akt and ERK signaling, and induction of the DR5/TRAIL pathway in tumor cells (See, e.g., Ishida et al., 2018; and Wagner et al., 2017, each of which is incorporated by reference with regard to such teaching).

Studies in human tumor xenograft mouse models revealed that dosing once weekly resulted in antitumor effects. Thus, the mechanism and kinetics of ONC206 suggest that ONC206 can exert efficacy with an infrequent dosing schedule. ONC206 has demonstrated antitumor efficacy in several preclinical cancer models and does not significantly impact the cell viability of normal human fibroblasts at doses that induce cell death in human cancer cells (See, e.g., Ishida et al., 2018; Wagner et al., 2017, each of which is incorporated by reference with regard to such teaching).

The preclinical safety profile of ONC206 has been demonstrated in non-GLP and GLP studies. At the highest doses tested, the drug did not achieve a maximum tolerated dose with oral administration in GLP studies. GLP studies with oral ONC206 revealed adverse events associated with the highest doses of ONC206 that were mild and reversible. The findings that were observed in both rats and dogs were decreased body weight and/or body weight gain (no effects on food consumption). These results support a NOAEL of 50 mg/kg in a Sprague-Dawley rat and 16.7 mg/kg in a Beagle dog. The human starting dose was calculated based on the NOAEL, which is approximately equivalent to a starting dose of 50 mg in dose escalation trials in adults.

Based on the initial non-clinical safety profile and its solubility, stability and in vivo activity, oral administration of ONC206 was selected as the route of administration in clinical trials. Preclinical studies suggest that ONC206 penetrates the intact blood brain barrier, achieves micromolar concentrations in the brain, and exhibits efficacy in DRD2-overexpressing tumor types such as neuroblastoma, pheochromocytoma, Ewing sarcoma, high-grade glioma, cholangiocarcinoma and medulloblastoma. These observations support the development of ONC206 as a potential treatment for multiple advanced solid tumors.

ONC206 is a member of the imipridone class of anti-cancer small molecules that share a unique tri-heterocyclic core chemical structure and target GPCRs. ONC206 has been shown to have broad-spectrum activity in vitro and found to have activity in tumor xenograft mouse models that warrants clinical investigation. The drug also has a favorable nonclinical safety and therapeutic PK profile in animals. The efficacy and safety of ONC206 is associated with its mechanism of action that involves antagonism of DRD2 that results in activation of ISR and induction of the DR5/TRAIL pathway, and results in antitumor efficacy in vitro and in vivo.

The initial safety profile of ONC206 is favorable relative to the observed anti-cancer activity. GLP studies with oral ONC206 revealed adverse events associated with the highest doses of ONC206 that were mild and reversible. The findings that were observed in both rats and dogs were decreased body weight and/or body weight gain (no effects on food consumption). The efficacy of ONC206 has been demonstrated in vitro and in vivo. Furthermore, ONC206 possesses an attractive profile that includes orally bioavailability, preclinical safety, high stability, water solubility and penetration of the blood-brain barrier.

ONC206 has not been previously tested in humans. The initial ONC206 clinical development program evaluates the safety and potential clinical activity of this compound as an orally administered treatment for adults with recurrent and rare primary central nervous system neoplasms. Oral administration was selected as the intended route of administration in clinical trials based on its bioavailability and anticancer activity in preclinical models with oral administration.

### Compositions

In one aspect, ONC206 may be provided as drug substance. In one embodiment, the drug substance may be provided in single-dose amber glass bottles to allow for the dissolution of ONC206 in water or other aqueous solvents or solvent systems, including pediatric drinks or other flavored solvent systems and purees, such as apple sauce or other fruit puree. Detailed instructions for the dissolution, labeling and dosing of the drug substance will be provided in the study protocol or study pharmacy manual.

In one aspect, ONC-206 may be provided as drug product. In one embodiment, the drug product may be comprised of hydroxypropyl methylcellulose (HPMC) capsules filled with the active ingredient, ONC206 dihydrochloride, intended for oral administration. In one embodiment, the drug product may be provided as a capsule with one or more of microcrystalline cellulose, sodium starch glycolate, and magnesium stearate.

An anticipated starting dose for a first-in-human clinical study is a 50 mg adult dose, as reflected below.

### Table 1: Starting Dose of ONC-206 in Study (Adult Dose)

TABLE 1
Starting Dose of ONC-206 in study (adult dose)

| | | Amount per | Amount |
|---|---|---|---|
| Component | Function | Capsule¹ | (% w/w) |
| ONC206 | Active | 50 mg | 100% |
| dihydrochloride | Pharmaceutical | | |
| | Ingredient | | |
| TOTAL | | 50 mg | 100% |

As noted herein, the amount per capsule reflects the equivalent amount based on the free base. The amount of ONC-206 di-HCl may be adjusted based on the salt, potency, and water content of the drug substance batches.

Storage Conditions: The capsules may be stored in the original closed container at room temperature (15 to 30° C.). Based on the current stability data, room temperature (25° C./60% Relative Humidity) will be used for the drug product storage. No shelf life has been established for this product at this point. However, representative clinical trial batches will be placed on stability. Any batches that are out of specifications will be removed from use.

Packaging: The product may be stored in a multi-dose container. The capsules are packaged in high-density polyethylene (HDPE) white opaque bottles, closed with an induction seal and capped with a white ribbed SecuRx® polypropylene (PPE) cap. The capsules are to be stored in the original closed container at room temperature (15 to 30° C.).

### Nonclinical Studies

### Nonclinical Pharmacology

### Summary

ONC206 has been shown to have broad-spectrum anti-cancer activity in vitro and found to have antitumor activity in xenograft mouse models of human cancer (Prabhu et al., 2017; Wagner et al., 2017, each herein incorporated by reference with regard to such teaching). The efficacy and safety of this molecule is associated with its mechanism of action that involves antagonism of DRD2 that results in activation of ISR and induction of the DR5/TRAIL pathway, which causes tumor-specific apoptosis resulting in antitumor efficacy in vitro and in vivo (Prabhu et al., 2017; Wagner et al., 2017, each herein incorporated by reference with regard to such teaching). This therapeutic mechanism does not impart cytotoxicity to normal cells (e.g. human fibroblasts) (Allen et al., 2015, herein incorporated by reference with regard to such teaching), which has been validated with ONC206 in normal human fibroblasts at efficacious nanomolar doses. Pharmacokinetic analysis in Sprague-Dawley rats and Beagle dog studies revealed micromolar plasma concentrations of ONC206. ONC206 has been safely administered in >50 mice for evaluating efficacy. Once weekly administration of 50 mg/kg (˜250 mg in a 60 kg human) ONC206 demonstrated robust inhibition of cholangiocarcinoma xenograft tumor growth. Single oral dosing in C57/B6 mice revealed an MTD at 300 mg/kg, 5-fold the efficacious dose. Administering up to 125 mg/kg ONC206 weekly in C57/B6 mice was well tolerated. Additionally, single dose and repeat dose non-GLP safety studies have been conducted in rats and dogs that have identified tolerable dose levels consistent with therapeutic thresholds.

GLP toxicology and safety pharmacology studies in Sprague Dawley rats and Beagle dogs were conducted, which included pulmonary, Functional Observational Battery (CNS), and cardiovascular readouts, that revealed a safety profile for ONC206 consistent with that of ONC201. The NOAEL in rats and dogs was the highest dose level tested, leading to a human equivalent starting dose of 50 mg ( 1/10 NOAEL) for adult dose escalation clinical trials based on standard allometric scaling and a body weight of 60 kg. The starting dose is expected to yield minimal subtherapeutic exposure for patients enrolled during dose escalation. ONC206 is produced as a dihydrochloride salt and all doses in the IND application have been corrected for the water and salt content to represent the free base dose.

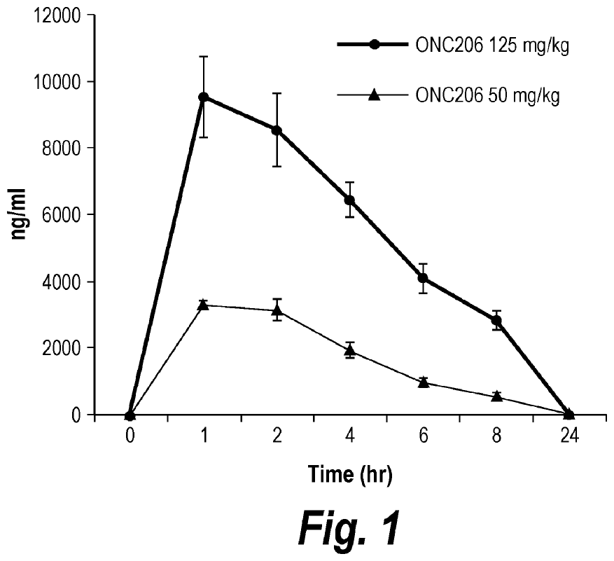

FIG. 1 illustrates a pharmacokinetic profile of ONC206 in Sprague Dawley rats following a single oral gavage dose (PO) of 50 and 125 mg/kg. 10000 ng/ml represents ˜20 μM.

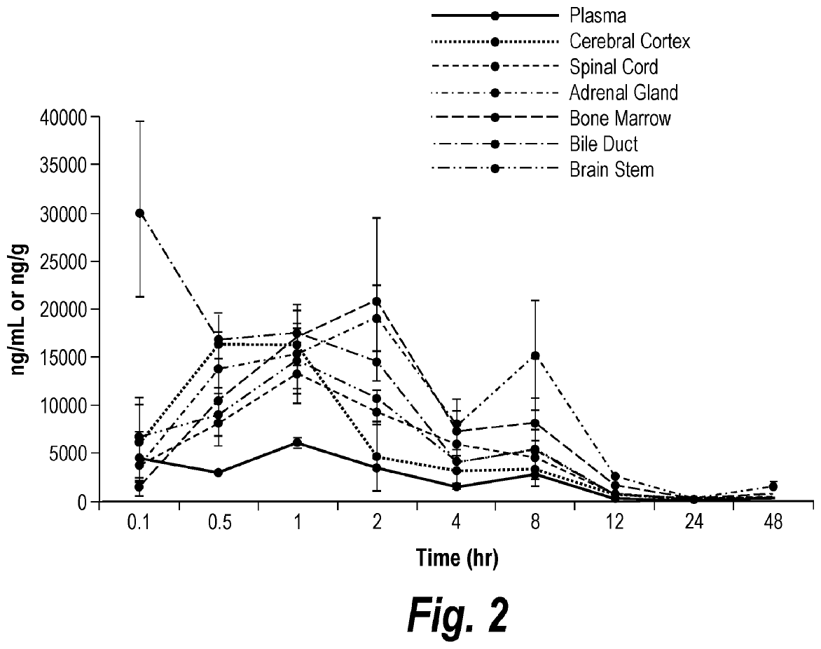

FIG. 2 illustrates rat biodistribution study of ONC206 with 50 mg/kg PO. Plasma and tissues concentrations depicted over time after ONC206 administration.

ONC206 exhibited a therapeutic PK profile upon oral dosing in rats, achieving a Cmax in the micromolar range (4-20 μM) with a terminal half-life of ˜6 hours (FIG. 1). Rat biodistribution studies revealed 5-10 fold higher ONC206 concentrations in target tissues of interest relative to plasma concentrations, including the adrenal gland (10-fold), bile duct (6-fold), brain (5-fold) and bone marrow (6-fold) (FIG. 2). This demonstrates that ONC206 can safely achieve systemic and target tissue concentrations in rats well beyond its nanomolar GI50.

### Mechanism of Action

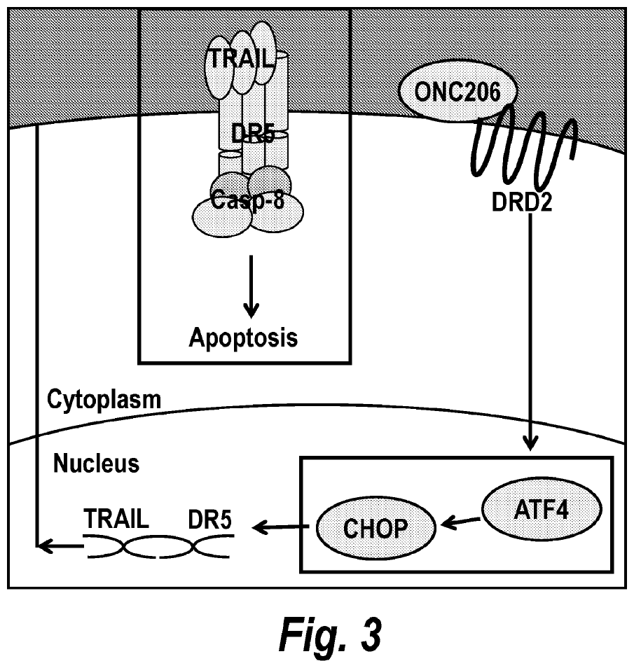

ONC206 is a member of the imipridone class of anti-cancer small molecules that share a unique tri-heterocyclic core chemical structure (Wagner et al., 2014, herein incorporated by reference with regard to such teaching) and selectively target GPCRs (Prabhu et al., 2017). GPCRs represent a superfamily of therapeutic targets that are underexploited for oncology and control several clinically validated signaling pathways for oncology, including ISR and Ras signaling (FIG. 3) (Lappano and Maggiolini, 2011, each herein incorporated by reference with regard to such teaching). Imipridones were created following the discovery of ONC201 (Allen et al, 2013), DRD2/3 antagonist (Madhukar et al., 2017, herein incorporated by reference with regard to such teaching) that has shown encouraging safety PK and PD in advanced cancers such as high grade gliomas (Arrillaga-Romany et al., 2017), where several patients achieved a RANO response, and endometrial cancer (Allen et al., 2016; Stein et al, 2017, each herein incorporated by reference with regard to such teaching), where several patients experienced prolonged PFS compared to historical control.

ONC206 is a selective antagonist of DRD2/3 that causes downstream action of ISR and DR5/TRAIL pathway and leads to tumor-specific apoptosis, demonstrated in its antitumor efficacy in vitro and in vivo (Prabhu et al., 2017; Wagner et al., 2017, each herein incorporated by reference with regard to such teaching).

FIG. 3 illustrates an ONC206 mechanism of action. ONC206 antagonizes DRD2 at the cell surface, resulting in activation of ISR involving ATF4/CHOP induction and upregulation of DR5 and TRAIL gene expression to induce apoptotic tumor cell death.

### Effects on Cancer and Normal Cells

ONC206 has demonstrated antitumor efficacy in several preclinical cancer models that include numerous human cancer cell lines in vitro and in vivo with the most pronounced efficacy in neuro-oncology and neuroendocrine tumors. Consistent with its mechanism of action, nanomolar in vitro efficacy of ONC206 was observed in neuroendocrine tumors and gliomas that included neuroblastoma, medulloblastoma, cholangiocarcinoma, pheochromocytoma cells, Ewings sarcoma, and glioma stem cells. Although ONC206 exhibits broad-spectrum activity across numerous preclinical solid tumors, its efficacy is pronounced at doses that do not appear to cause adverse effects in normal cells (Table 4).

TABLE 4
ONC206 differential in vitro activity in malignant
versus normal cells. (A) GI50 for ONC206 in Ewings sarcoma
and normal fibroblasts cells at indicated time points.
GI50 (μM)

| | ONC206 | | |
|---|---|---|---|
| | 24 h | 48 h | 72 h |
| MHH-ES-1 | >5 | 4.98 | 0.61 |
| (Ewings) | | | |
| CADO-ES I | >5 | 4.93 | 0.55 |
| (Ewings) | | | |
| HFF-1 (normal) | >5 | >5 | >5 |

### In Vitro Mono-Agent

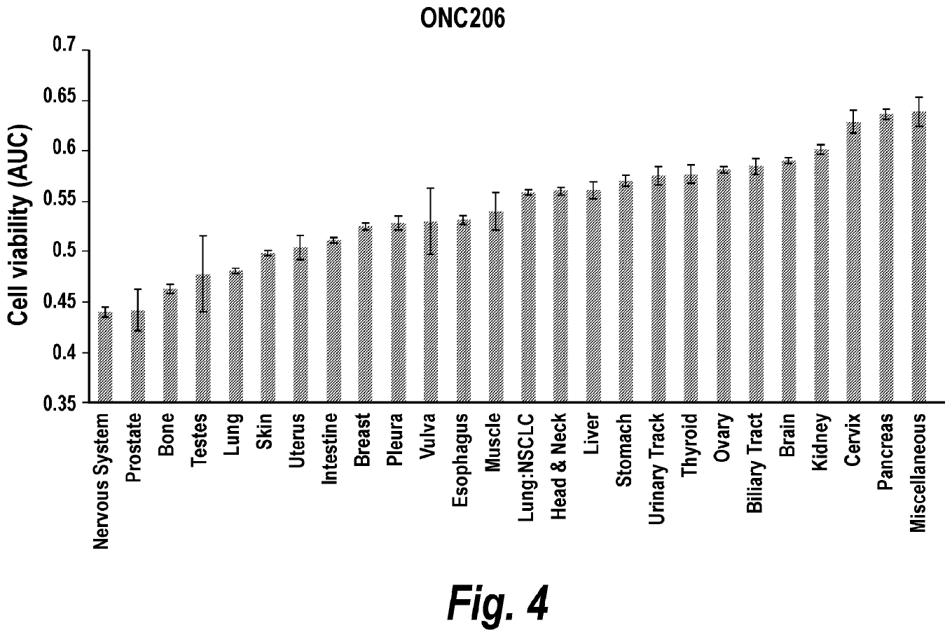

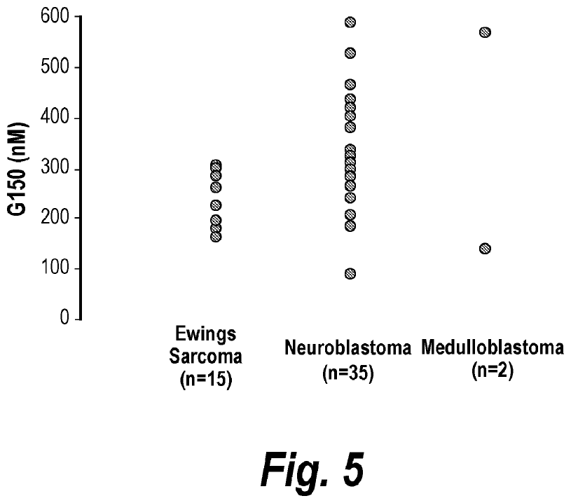

ONC206 demonstrated broad spectrum anti-cancer efficacy in vitro across most solid tumor types tested in a panel of >1,000 human cancer cell lines with nervous system tumors emerging as most responsive (FIG. 4 and FIG. 5). Consistent with its mechanism of action, nanomolar in vitro efficacy of ONC206 was observed in neuroendocrine tumors and gliomas that included neuroblastoma, medulloblastoma, cholangiocarcinoma, pheochromocytoma cells, Ewings sarcoma, and glioma stem cells.

As shown in FIGS. 4-7, ONC206 has been tested for in vitro efficacy in human cancer cell lines.

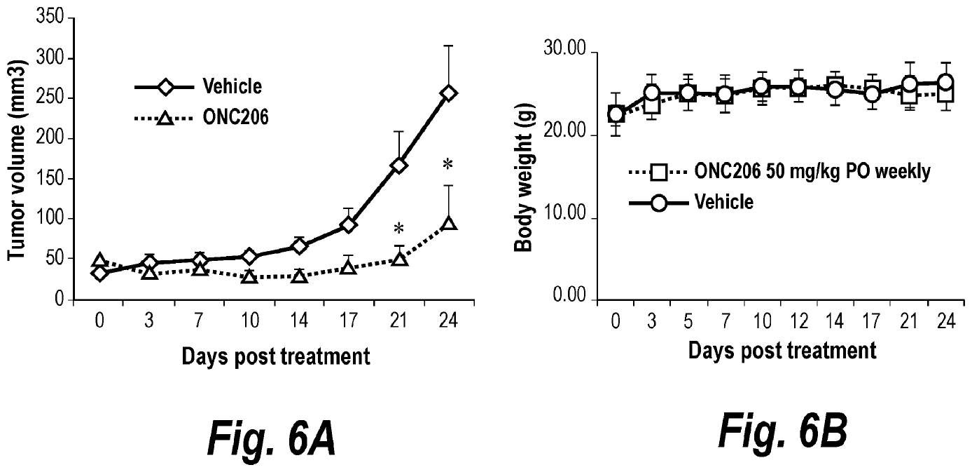

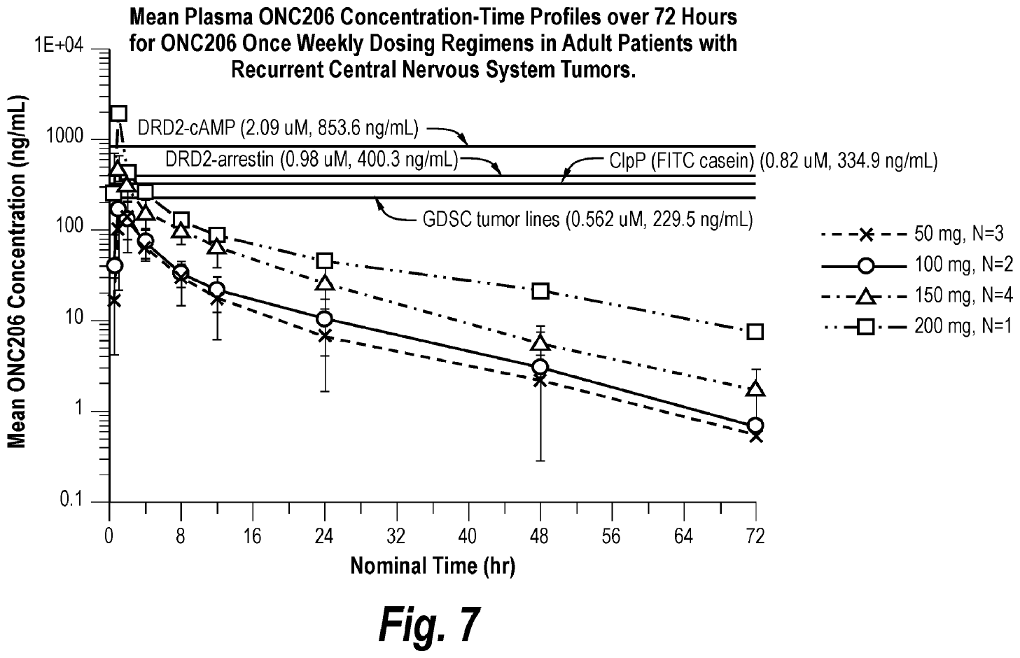

FIG. 4 illustrates in vitro sensitivity of >1000 Genomics of Drug Sensitivity in Cancer (GDSC) human cancer cell lines to ONC206 (72 h) averaged and organized by tumor type. The results are shown as completeness of ONC206 response quantified as the average area under the curve (AUC) in the dose-response cell viability curve among all cell lines in each tumor type. Error bars represent standard error of mean. FIG. 5 illustrates average GI50 with 72 hour ONC206 (0.078-20 μM) treatment in a panel of Ewings sarcoma, neuroblastoma and medulloblastoma cell lines in the GDSC screen. FIGS. 6 and 7 illustrate representative dose-response curve (n=2) of ONC206 in MC-IXC neuroblastoma and PC12 pheochromocytoma cells.

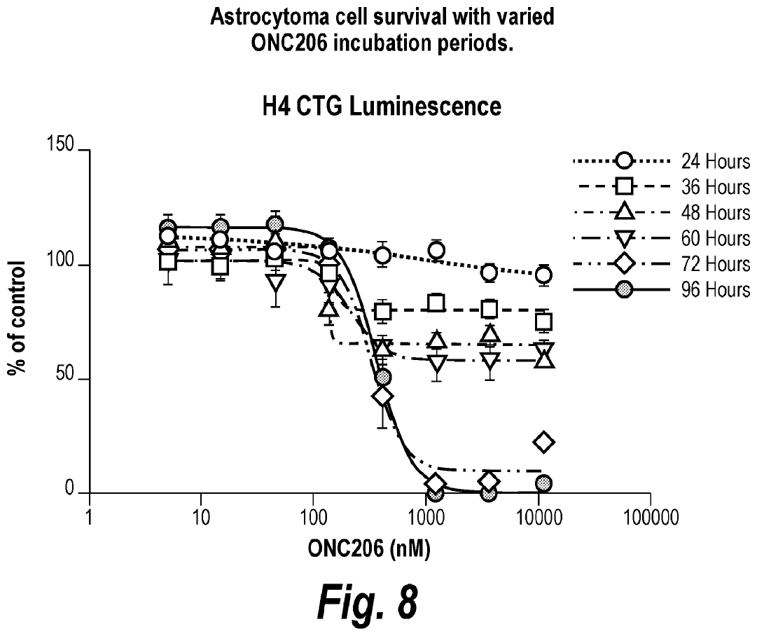

FIG. 8 illustrates in vitro efficacy of ONC206 in human glioma stem cells. Cell viability dose response curves for ONC206 in patient-derived glioma stem cells (left) (GI50<100 nM) and bulk tumor cells (right) (GI50 100-500 nM). (Jung et al., 2018)

### In Vivo Mono-Agent

Nonclinical mouse model data revealed inhibition of tumor growth with weekly oral dosing of 50 mg/kg ONC206 in subcutaneous xenografts of cholangiocarcinoma (FIG. 7) without body weight loss. Given the ˜5 hour half-life of ONC206 and its prolonged activity with weekly dosing, the pharmacokinetics do not appear to be directly related to the efficacy of ONC206. Disconnect between PK and PD has also been observed with ONC201, the parent compound of ONC206 that also targets DRD2. Based on these observations, weekly dosing will be used in the first-in-human study of ONC206.

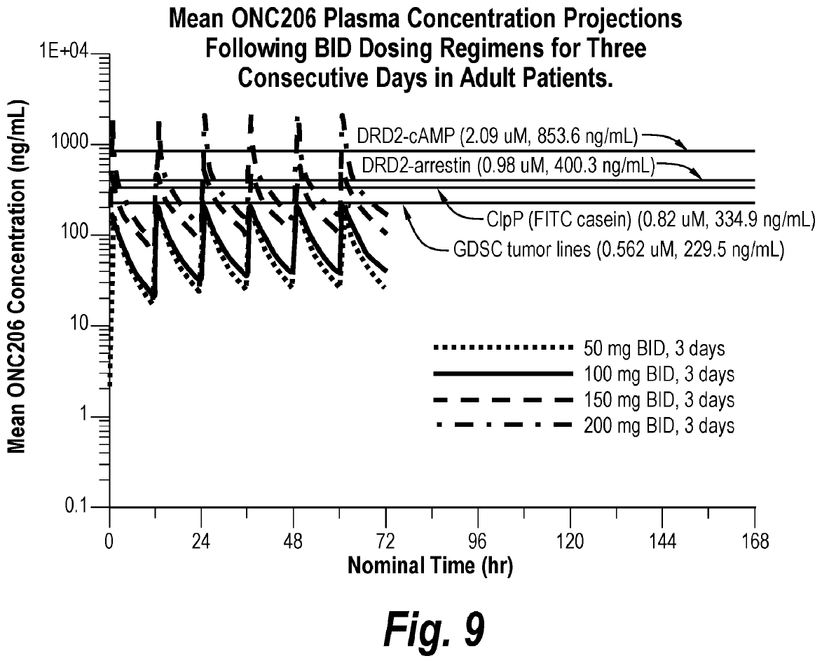

FIG. 9 illustrates in vivo antitumor efficacy of ONC206 at 50 mg/kg once a week without body weight loss. (A) Tumor volume of HuCCT1 xenografts in athymic nude mice and (B) associated body weight following continuous treatment with ONC206 50 mg/kg PO and vehicle once a week (n=6). * p<0.05

### Nonclinical Toxicology

All doses and concentrations of ONC206 are presented as free base and are corrected for dihydrochloride and water contents.

### Non-GLP Safety Studies

Single dose and repeat dose non-GLP safety studies have been conducted in mice, rats and dogs that have identified tolerable dose levels consistent with therapeutic thresholds. These studies recorded clinical observations, body weight, food consumption, and gross findings at sacrifice.

### Mouse Repeat Dose Toxicology

A repeat dose oral non-GLP toxicity study was conducted using experimentally naïve female C57/B16 mice (6-8 weeks old). Animals were dosed via oral gavage. Animals were dosed for 15 days either once per week (Day 1, 8 and 15), three times per week (Day 1, 3, 5, 8, 10, 12 and 15) or daily. Administering 125 mg/kg ONC206 (>2-fold efficacious dose, equivalent to 625 mg in human) daily resulted in significant body weight loss after a week that prompted euthanasia. Administering 125 mg/kg ONC206 three times a week caused tolerable body weight loss that was not observed with weekly administration at the same dose. Blood draw for PK was done 1 h post day 7 for the daily group and 1 h post day 15 for the other 2 groups. The concentrations observed were 6.5, 10.9 and 5 μM for the once per week, three times per week and daily dosing groups respectively.

### Sprague-Dawley Rat Single Dose Toxicology

A single dose oral non-GLP toxicity study of ONC206 in Sprague Dawley rats used ten experimentally naïve rats (5 males and 5 females) that were assigned to treatment groups as shown in the Table below. The study groups were dosed single oral doses of ONC206 at the chosen dose level in succession using a dose escalation study design.

TABLE 5
Treatment groups for non-GLP single dose
toxicity studies in Sprague-Dawley rats

| | Dose | | | | |
|---|---|---|---|---|---|
| | Level | Concen- | Dose | Number of | |
| | (mg/kg/ | tration | Volume | Animals | |
| Group | day) | (mg/ml) | (ml/kg) | Male | Female |
| 1. ONC206 | 61.3 | 6.13 | 10 | 1 | 1 |
| 2. ONC206 | 184 | 18.4 | 10 | 1 | 1 |
| 3. ONC206 | 122.6 | 12.26 | 10 | 1 | 1 |
| 4. ONC206 | 81.8 | 8.18 | 10 | 1 | 1 |
| 5. ONC206 | 102.2 | 10.22 | 10 | 1 | 1 |

No mortality was observed throughout the study in the test article treated animals. Animals exhibited clinical signs at 102.2, 122.6, and 184 mg/kg. No clinical signs were noted at 61.3 or 81.8 mg/kg.

Terminal necropsy on Day 8 revealed no visible lesions in any of the animals treated with ONC206 at 61.3, 81.8, 102.2, 122.6, or 184 mg/kg. Based on the results of this study, the NOAEL of ONC206 when administered orally to the rat was determined to be less than or equal to 102.2 mg/kg and the maximum tolerated dose was determined to be less than or equal to 122.6 mg/kg.

PBS was used as the vehicle for ONC206 in the non-GLP rat single dose study. The 61.3, 122.6, and 184 mg/kg dose levels were clear liquids with small (very small minute amount) of clear particles/fibers. The 81.8 and 102.2 mg/kg dose levels were clear colorless liquids. Following this study, sterile water for injection was used for all subsequent studies as the solubility was better in water than PBS.

### Conclusion

The NOAEL following single-dose administration of ONC206 to Sprague-Dawley rats by oral gavage was determined to be less than or equal to 102.2 mg/kg.

### Sprague-Dawley Rat Repeat Dose Toxicology

A repeat dose oral non-GLP toxicity study was also conducted using experimentally naïve Sprague Dawley rats that were assigned to treatment groups as shown in Table 6 below.

TABLE 6
Treatment groups for non-GLP repeat dose
toxicity study in Sprague Dawley rats

| | Dose | Concen- | Dose | Number of | |
|---|---|---|---|---|---|
| | Level | tration | Volume | Animals/Sex | |
| Group | (mg/kg) | (mg/ml) | (mv/kg) | Female | Male |
| 1. ONC206 Weekly | 50 | 5 | 10 | 2 | 2 |
| 50 mg/kg | | | | | |
| 2. ONC206 Weekly | 100 | 10 | 10 | 2 | 2 |
| 100 mg/kg | | | | | |
| 3. ONC206 Weekly | 25 | 2.5 | 10 | 2 | 2 |
| 25 mg/kg | | | | | |
| 4. ONC206 Weekly | 75 | 7.5 | 10 | 2 | 2 |
| 75 mg/kg | | | | | |
| 5. ONC206 Weekly | 6 | 0.6 | 10 | 2 | 2 |
| 6 mg/kg | | | | | |
| 6. ONC206 Weekly | 60 | 6 | 10 | 4 | 2 |
| 60 mg/kg | | | | | |

The Animals were dosed via oral gavage. Animals were dosed once per week for 28 days (Days 1, 8, 15, 22, 28).

Male and female Sprague Dawley rats tolerated a weekly dose of 6 mg/kg of ONC206 when administered by oral gavage as evidenced by no early mortality/morbidity and no clinical signs (aside from piloerection seen in one male on Day 29 only). However, an overall decrease of food consumption values and body weights were observed over the course of the study (food consumption decrease primarily in the males and body weight decrease primarily in the females). Some hematology and serum chemistry values were outside of normal historical limits (glucose increased and hemoglobin and hematocrit decreased in the males and females). Gross necropsies of the animals on Day 29 did not show any visible lesions. A decrease in the number of zymogen granules in pancreatic acinar cells was observed in 0 out of 2 males and 0 of 2 females at 6 mg/kg (Group 5). Intensity of this change was usually of moderate degree.

Male and female Sprague Dawley rats tolerated a weekly dose of 25 and 50 mg/kg of ONC206 when administered by oral gavage as evidenced by no early mortality/morbidity; however, clinical observations such as abnormal gait, decreased activity, piloerection, and decreased muscle tone were observed (primarily in the females, males appeared normal throughout the study until one male presented with decreased activity, abnormal gait, and piloerection on Day 29); an overall decrease of food consumption values and body weights were observed over the course of the study (primarily in the females), and hematology and serum chemistry values were outside of normal historical limits (red blood cells, hemoglobin, hematocrit, monocyte percentage all decreased and glucose increased in the males; hemoglobin, hematocrit decreased and aspartate amino transferase, triglycerides, and glucose increased in the females). Gross necropsies of the animals on Day 29 did not show any visible lesions. At the Day 29 time point, for the tissues evaluated microscopically, test article-related observations included decreased zymogen granules in pancreatic acinar cells in 25 and 50 mg/kg ONC206 female animals. This finding appeared to be non-adverse.

Male and female Sprague Dawley rats did not tolerate a weekly dose of 60 mg/kg of ONC206 when administered by oral gavage as evidenced by an early death in one female on Day 13 (though the animal appeared normal from Days 1-12). The necropsy of this animal revealed dark lung, spleen dark at the tip, liver mottled with pale areas and dark at edges, dark kidneys, stomach and duodenum distended with air. For the single early death female in Group 6 (Animal 2686), there was widespread autolysis of multiple organs to include stomach, duodenum, jejunum, ileum, cecum, colon, rectum, kidneys, liver, spleen, mesenteric lymph node, and bone marrow (sternum). The degree of autolysis precluded microscopic evaluation of these organs. Macroscopic findings for this animal including dark areas in the liver, spleen, and kidneys as well as the stomach and duodenum distension with air were attributable to autolytic/post mortem changes. The remaining macroscopic observation of dark lungs documented by the Testing Facility correlated microscopically to congestion which was an agonal change. A cause for mortality could not be determined for this animal.

Male and female Sprague Dawley rats did not tolerate a weekly dose of 75 mg/kg of ONC206 when administered by oral gavage as evidenced by an early death in one female on Day 12 and a moribund sacrifice in one female on Day 12. Hematology values (serum chemistry was not able to be analyzed due to insufficient plasma quantity) for the female that was moribund sacrificed were outside of the normal historical limits (decreased platelets, lymphocyte percent, and number of and percent of reticulocytes as well as increased neutrophil number). No visible lesions were noted at necropsy for these two animals. The morbidity/mortality of the two Group 4 female animals (75 mg/kg ONC206) was associated with test article-related microscopic findings of single cell necrosis of hepatocytes in the liver, bone marrow hypocellularity, and single cell necrosis of acinar cells and/or decreased numbers of zymogen granules in acinar cells of the pancreas. For Group 4 female Animal 1868, decreased numbers of lymphocytes were noted in the spleen (periarteriolar lymphatic sheath) and mesenteric lymph nodes (cortex/paracortex). The morphology of single cell hepatocellular necrosis in these Group 4 female animals was suggestive of apoptosis and occurred primarily with a midzonal to periportal distribution. Similarly, the observed single cell necrosis of pancreatic acinar cells was suggestive of apoptosis. Hypocellularity involving the bone marrow appeared to involve a decrease in cells of both the erythroid and myeloid cell series while megakaryocytes looked to be preserved.

Male and female Sprague Dawley rats did not tolerate a weekly dose of 100 mg/kg of ONC206 when administered by oral gavage as evidenced by mortality in one female on Day 11 and one female on Day 13, numerous clinical observations such as abnormal gait and stance, decreased activity, piloerection, mild circling, thin hair coat, decreased muscle tone (primarily in the females), an overall decrease of food consumption values and body weights over the course of the study, and hematology and serum chemistry values that were outside of normal historical limits (red blood cells, hemoglobin, hematocrit all decreased and blood urea nitrogen increased) in the males. Gross necropsies of the animals that survived through Day 29 did not show and visible lesions. Gross necropsy of the female found dead on Day 11 did not reveal any visible lesions and gross necropsy of the female found dead on Day 13 revealed dark areas throughout all lobes of the lungs and liver. The morbidity/mortality of two Group 2 female animals (100 mg/kg ONC206) was associated with test article-related microscopic findings of single cell necrosis of hepatocytes in the liver, bone marrow hypocellularity, and single cell necrosis of acinar cells and/or decreased numbers of zymogen granules in acinar cells of the pancreas.

### Conclusion

The NOAEL following repeat-dose administration of ONC206 to Sprague-Dawley rats by oral gavage was not reported. At 6 mg/kg no notable clinical signs observed. At 25 mg/kg, clinical observations such as abnormal gait, decreased activity, piloerection, and decreased muscle tone were observed. At 50 mg/kg, less persistent and shorter duration clinical signs when compared to the 100 mg/kg dose group, and no deaths observed. At 60, 75, and 100 mg/kg associated with deaths and clinical observations.

### Beagle Dog Single Dose Toxicology

A single dose oral non-GLP pyramid toxicity study was conducted in beagle dogs using two experimentally naïve Beagle dogs (one male and one female) assigned to treatment groups as shown in Table 7 below. A dose volume of 20 mL/kg was utilized for each oral dose.

TABLE 7
Treatment groups for non-GLP pyramid
toxicity study in Beagle dogs

| | |
|---|---|
| Dose* | Number of Animals |

| | (mg/kg/day) | Male | Female |
|---|---|---|---|
| Dose #1 | 12.5 | 1 | 1 |
| Dose #2 | 25 | 1 | 1 |
| Dose #3 | 19 | 1 | 1 |
| Dose #4 | 12.5 | 1 | 1 |

On Day 1, 12.5 mg/kg of ONC206 (initial dose) was administered by oral gavage to the two naive dogs (1 male and 1 female). After a washout period of approximately 7 days, ONC206 at 25 mg/kg (dose 2) was administered to the same two dogs on Day 8. ONC206 at 19 mg/kg (dose 3) was dosed on Day 15 to both dogs. The final dose, dose #4 at 12.5 mg/kg was administered on Day 22 to both dogs.

There were no early deaths or terminations during the study. There were no clinical findings on Days 1-5 following the initial dose of 12.5 mg/kg; however, a small amount of soft feces was observed in the female on Day 6 while the male appeared normal. By Day 7, both animals appeared normal and continued to appear normal through Day 8 (prior to dose #2). There were clinical findings following dose 2 at 25 mg/kg. At 1 hour post dose, both animals appeared normal; however, at the unscheduled observation time point of 2 hours post dose, a medium amount of emesis containing food particles was observed in the male while the female was observed to have a small amount of salivation and a medium amount of soft feces. An unscheduled observation was also made approximately 6 hours post dose where the female was observed to have a medium amount of salivation and the male was observed to have a medium amount of clear yellow emesis containing food particles and a small amount of mucoid soft feces. At approximately ten hours post dose, unscheduled observations of a medium amount of yellow, green, mucoid watery feces and a small amount of mucoid soft feces were observed in the male. By Day 9, the male appeared normal while the female exhibited a small amount of soft feces. On Day 10, both animals appeared normal. On Day 11, the female continued to appear normal while the male exhibited a small amount of loose feces. From Days 12-14, both animals appeared normal. On Day 15, prior to dose 3, the male appeared normal while the female was observed to have a medium amount of soft feces.

On Day 15, after a de-escalated dose of 19 mg/kg (Dose 3), at 1-2 hour post dose, the male had a small amount of salivation while the female had a medium amount of salivation. An unscheduled observation was also made approximately 6 hours post dose where the female was observed to have a small amount of mucoid loose feces and the male was observed to have a medium amount of mucoid loose feces. By Day 16, both animals appeared normal and they continued to appear normal through Day 22 (prior to dose 4).

No clinical observations were observed following dose 4 at 12.5 mg/kg, all animals appeared normal from 1-2 hours post dose through Day 29.

In conclusion, the test article ONC206 caused toxicological effects when administered to beagle dogs orally via gavage at 19 or 25 mg/kg. Based on post-dose clinical signs, changes in body weights, food consumption, the NOAEL was determined to be less than or equal to 12.5 mg/kg.

### Conclusion

The NOAEL following single-dose administration of ONC206 to Beagle dogs by oral gavage was determined to less than or equal to 12.5 mg/kg.

TABLE 8
Treatment groups nor non-GLP repeat
dose toxicity study in Beagle dogs

| | Dose | Concen- | Dose | Number of | |
|---|---|---|---|---|---|
| | Level | tration | Volume | Animals/Sex | |
| Group | (mg/kg) | (mg/ml) | (ml/kg) | Female | Male |
| 1. ONC206 Weekly Low | 8.3 | 0.83 | 10 | 1 | 1 |
| 2. ONC206 Weekly Mid | 12.5 | 1.25 | 10 | 1 | 1 |
| 3. ONC206 Weekly Mid- | 16.7 | 1.67 | 10 | 1 | 1 |
| High | | | | | |
| 4. ONC206 Weekly High | 20 | 2 | 10 | 1 | 1 |

Male and female Beagle Dogs tolerated weekly doses of 8.3 and 12.5 mg/kg of ONC206 when administered by oral gavage as evidenced by no early mortality/morbidity, no clinical signs, relatively stable food consumption values (though decreases were observed following each dose but improvement was seen prior to following dose), and relatively stable body weights. Hematology and serum chemistry values were relatively within historical limits (increased alanine amino transferase was observed in female in Group 1). Gross necropsies of all animals in Groups 1 and 2 on Day 29 did not reveal any notable findings.

Male and female Beagle Dogs receiving weekly doses of 16.7 and 20 mg/kg of ONC206 when administered by oral gavage did not tolerate the test article as well as the lower dose groups. No early mortality/morbidity was observed. Test article related effects noted at these two highest dose levels included occasional emesis and salivation after dosing. The clinical signs were short acting and not observed on non-dosing days. Lower food consumption was observed in the two highest dose levels after dosing but quickly resolved. This lower food consumption indirectly contributed to an overall lower final body weight for the animals in the highest dose groups when compared to their starting and ending body weights for the lower dose animals. Some hematology and serum chemistry values were outside of normal historical limits (increased aspartate amino transferase in female in Group 3, increased alanine amino transferase in female in Group 4, and increased total bilirubin in Group 3 female). Gross necropsies of all animals in Groups 3 and 4 on Day 29 did not reveal any notable findings.

There were no test article-related microscopic observations. Mild chronic focal inflammation of the alveolar and bronchial areas of the lung was present in three dogs. The findings were considered to be secondary to minor aspiration of the dosing solution, and not a direct effect of ONC206. There were no test article-related pathology findings.

### Conclusion

The NOAEL following repeat-dose administration of ONC206 to Beagle dogs by oral gavage was not reported. At 8.3 and 12.5 mg/kg no notable findings. At 16.7 and 20 mg/kg lower food consumption and lower body weight was observed. Occasional emesis and salivation observed after dosing.

TABLE 9
treatment groups for GLP repeat dose
toxicity study in Sprague Dawley rats

| | Dose | | | | |
|---|---|---|---|---|---|
| | Level* | Concen- | Dose | Number of | |
| | (mg/kg/ | tration* | Volume | Animals** | |
| Group | day) | (mg/mL) | (mL/kg) | Male | Female |
| 1. Control (vehicle) | 0 | 0 | 10 | 15 | 15 |
| 2. ONC206 Low- | 5 | 0.5 | 10 | 15 | 15 |
| dose | | | | | |
| 3. ONC206 Mid-dose | 25 | 2.5 | 10 | 15 | 15 |
| 4. ONC206 High-dose | 50 | 5.0 | 10 | 15 | 15 |

The dose levels and concentrations shown in the table represent actual API (free base). Also, a targeted 10 animals/sex/group were euthanized on Day 23. A targeted 5 animals/sex/group remained on study, untreated, for a one week recovery period and were euthanized on Day 29.

Male and female Sprague Dawley rats tolerated ONC206 when administered by oral gavage, once weekly for three weeks, at 0, 5, 25 or 50 mg/kg.

Evaluation of clinical signs and functional observational battery did not reveal any treatment related signs. No significant ocular lesions were noted by the study ophthalmologist.

Animals in the 25 and 50 mg/kg group had lower body weights than the controls at the completion of the dosing phase on Day 22, but the differences in group mean body weights were minimal and were demonstrated to be reversible during the recovery phase.

There were sporadic differences in group mean food consumption that did not appear to be related to treatment.

At the terminal clinical pathology evaluation on Day 23, there were minor increases in hematology and serum chemistry parameters related to treatment, including increases in relative monocytes (25 and 50 mg/kg), glucose (25 and 50 mg/kg), sodium (25 and 50 mg/kg), chloride (25 and 50 mg/kg), and alkaline phosphatase (50 mg/kg). All of these findings were reversible.

At necropsy on Days 23 and 29, there were no macroscopic tissue observations or differences in organ weights attributed to test article related toxicity.

One unscheduled death occurred in the 50 mg/kg group. A male animal was found dead on Day 22 following the final dose. A cause of death could not be determined. The mortality was limited to one animal out of a total of forty-eight total animals in the 50 mg/kg treatment groups. Given the low incidence of mortality, and the fact that there was no evidence of adverse test article related toxicity noted for any of the animals in the 50 mg/kg group, the death was determined to be spontaneous.

The NOAEL and HNSTD for ONC206 was >50 mg/kg.

### Conclusion

Based on the results of this study, the NOAEL and HNSTD following oral administration of ONC206 at repeat doses of 5, 25, and 50 mg/kg to Sprague-Dawley rats is considered to be 50 mg/kg

TABLE 10
treatment groups for GLP repeat dose toxicity study in Beagle dogs

| | Dose | Concen- | Dose | Number of | |
|---|---|---|---|---|---|
| | Level* | tration* | Volume | Animals** | |
| Group | (mg/kg/day) | (mg/ml) | (ml/kg) | Male | Female |
| 1. Control (vehicle) | 0 | 0 | 10 | 5 | 5 |
| 2. ONC206 Low-dose | 1.7 | 0.17 | 10 | 5 | 5 |
| 3. ONC206 Mld-dose | 8.3 | 0.83 | 10 | 5 | 5 |
| 4. ONC206 High-dose | 16.7 | 1.67 | 10 | 5 | 5 |
| 5. ONC206 IV dose | 8.3 | 0.83 | 10 | 3 | 3 |

The dose levels and concentrations shown in the table represent actual API (free base).
3 animals/sex/group in Groups 1-4 were euthanized on Day 23.
2 animals/sex/group in Groups 1-4 remained on study, untreated, for a 6 day recovery period and were euthanized on Day 29.
3 animals/sex in Group 5 were euthanized on Day 29.

Animals in Groups 1-4 (5/sex/group) were dosed once daily on Days 1, 8, 15 and 22 via oral gavage at dose levels of 0, 1.7, 8.3 and 16.7 mg/kg/day. Animals in Group 5 (3/sex/group) were dosed once on Day 1 by intravenous infusion (˜30 minutes) via the cephalic vein using an appropriately sized syringe, indwelling catheter and calibrated infusion pump.

Male and female beagle dogs tolerated ONC206 when administered by oral gavage, once weekly for three weeks (total of 4 doses), at 0, 1.7, 8.3 or 16.7 mg/kg. In addition, male and female dogs tolerated ONC206 when administered as a single dose via intravenous infusion (˜30 minutes) at 8.3 mg/kg. Minor effects associated with ONC206 were demonstrated to be reversible during the recovery period.

No test article related clinical signs were observed. There were no effects on food consumption throughout the dosing and recovery phases. No significant ocular lesions were observed at the terminal and recovery ophthalmology examinations.

There were no toxicological effects on cardiac rhythm or ECG morphology based on the electrocardiograms evaluated during this study.

There were test article related effects on body weight gains. Animals dosed at 16.7 mg/kg had low weight gains, as compared to the controls. Some of the female animals had cumulative weight loss over the course of the 3-week dosing phase, ranging from 3 to 9% total weight loss, relative to starting weight. The effects on body weight gain appeared to reverse during the one week recovery phase. Given the relatively slow rate of weight loss and evidence of reversibility, these findings were not considered to be adverse.

Clinical pathology evaluations revealed minor findings that may have been related to test article effects but were not indicative of overt toxicity. Female animals given ONC206 intravenously at 8.3 mg/kg had minor increases in relative lymphocytes and decreases in chloride on Day 23. These findings were associated with individual animal values that were only slightly outside the range of historical control data and were not considered to be adverse. There were no test article changes in coagulation, erythrocyte morphology or urinalysis parameters.

There were no ONC206-related microscopic pathology changes.

The NOAEL and HNSTD for ONC206 when administered by oral gavage, once weekly for three weeks, is 16.7 mg/kg. The NOAEL for ONC206 when administered by a single intravenous infusion (˜30 minutes) is 8.3 mg/kg. This dose and route was administered to determine the bioavailability

### Conclusions

Based on the results of this study, the NOAEL and HNSTD following oral administration of ONC206 at repeat doses of 1.7, 8.3, and 16.7 mg/kg to Beagle dogs is considered to be greater than or equal to 16.7 mg/kg

### Pharmacokinetics/Toxicokinetics

In rats, exposure to ONC206 following oral gavage dosing at 5, 25, and 50 mg/kg/day ONC206 was dose-dependent and approximately dose-proportional. Exposure to ONC206 was slightly greater in female rats after oral gavage dose administration and after a 30-minute intravenous infusion. Absolute oral bioavailability following 5, 25, and 50 mg/kg/day was 25% to 67%, 51% to 84%, and 68% to 96%, respectively. The oral Tmax was observed at the first measured timepoint (0.5 hours) which suggests that the drug was rapidly absorbed. Elimination of ONC206 was similar following single oral and intravenous administrations. Plasma T½, e ranged from 2.0 to 6.1 hours in all 8 profiles. Clearance ranged from 2.7 to 8.4 L/hr/kg in 7 of 8 profiles. Volume of distribution ranged from 8.9 to 56.2 L/kg in all 8 profiles. There did not appear to any great differences in ONC206 absorption, exposure, and elimination following single and multiple oral dosing.

In dogs, exposure to ONC206 following oral gavage dosing at 1.7, 8.3, and 16.7 mg/kg/day was dose-dependent and greater than dose-proportional across all dose levels for male and female dogs. Exposure to ONC206 was slightly greater in female dogs after a single oral gavage mid dose administration on Day 1 and after four weekly oral doses at the mid and high dose levels on Day 22; exposure to ONC206 was similar after a 30-minute intravenous infusion to male and female dogs. Absolute oral bioavailability following 1.7, 8.3, and 16.7 mg/kg/day was ˜5%, ˜23%, and ˜52%, respectively. The oral Tmax was observed at the first measured timepoint (0.5 hours) which suggests that the drug was rapidly absorbed. Mean oral plasma T½, e values ranged from 0.7 to 7.1. Oral clearance moderately variable with mean CI/f values ranging from 5.0 to 101 L/hr/kg. Mean oral volume of distribution values ranged from 25.2 to 259 L/kg. There did not appear to any real differences in ONC206 absorption, exposure, and elimination following single and multiple oral dosing.

### Safety Pharmacology Studies

### Respiratory

A respiratory study was conducted as a component of the GLP safety rat study. Twenty-four (6/group) male rats were trained on two occasions in the head-out plethysmograph chamber for approximately 15 minutes each, before the day of the experiment. On the day of dosing, each animal was placed in its plethysmograph chamber and baseline values were obtained for 5 minutes following an approximately 5-minute stabilization period. The rats were then removed from the chamber and dosed by oral gavage as per the following schedule:

TABLE 11
respiratory function treatment groups and corresponding doses

| | | Dose | Volume | Concentration |
|---|---|---|---|---|
| Group | Treatment | (mg/kg) | (mL/kg) | (mg/mL) |
| 1 | Vehicle control | 0 | 5 | 0 |
| 2 | ONC206 (low) | 5 | 5 | 1.0 |
| 3 | ONC206 (mid) | 25 | 5 | 5 |
| 4 | ONC206 (high) | 50 | 5 | 10 |

Twenty four (6/group) male rats were trained on two occasions in the head-out plethysmograph chamber for approximately 15 minutes each, before the day of the experiment. On the day of dosing, each animal was placed in its plethysmograph chamber and baseline values were obtained for 5 minutes following an approximately 5-minute stabilization period. The rats were then removed from the chamber and dosed by oral gavage. Following dosing, each animal was returned to its designated plethysmograph chamber and the respiratory parameters were recorded at 15 minutes (±3 min), 1, 2, and 4 hours (±5 minutes) following dose administration of test article/vehicle. Animals were allowed to stabilize in the plethysmograph for at least 5 minutes before each reading was taken. The following parameters were acquired, recorded and analyzed using DSI Dataquest Open ART (v. 2.3) & PONEMAH Physiology Platform (v. 4.2.0):
Respiratory rate
Tidal Volume
Minute Volume

The oral administration of ONC206 at doses of 5, 25 and 50 mg/kg did not induce any significant effects on respiratory rate, tidal volume or minute volume compared to the vehicle in conscious male rats.

In conclusion, no significant effects on respiratory rate at 5, 25 and 50 mg/kg were observed.

### Cardiovascular

A cardiovascular study was conducted as a component of the GLP safety dog study. Animals were randomly assigned according to study protocol and by gender to five groups of 3-5 dogs and dosed via oral gavage once weekly for three weeks (total of 4 doses).

ECGs were obtained from all study animals in right lateral recumbency prior to treatment initiation (Baseline), following dose administration on Day 1, following the final dose on Day 22, and on Day 28 of the recovery phase. All recordings were made at 50 mm/sec paper speed. Recordings were made using limbs leads I, II, III, aVR, aVL, aVF, and two chest leads V1 and V2. For each trace, the recording was visually evaluated by the board-certified cardiologist for rhythm disturbances and changes in the general configuration of the complexes.

TABLE 12
Cardiovascular study treatment groups and corresponding doses

| | Dose | | | | |
|---|---|---|---|---|---|
| | Level* | Concen- | Dose | Number of | |
| | (mg/kg/ | tration* | Volume | Animals** | |
| Group | day) | (mg/ml) | (ml/kg) | Males | Females |
| 1. Control (vehicle) | 0 | 0 | 10 | 5 | 5 |
| 2. ONC206 Low-dose | 1.7 | 0.17 | 10 | 5 | 5 |
| 3. ONC206 Mid/low- | 8.3 | 0.83 | 10 | 5 | 5 |
| dose | | | | | |
| 4. ONC208 Mid/high- | 16.7 | 1.67 | 10 | 5 | 5 |
| dose | | | | | |
| 5. ONC206 IV dose | 8.3 | 0.83 | 10 | 3 | 3 |

The dose levels and concentrations represent actual API.
3 animals/sex/group in Groups 1-4 were euthanized on Day 23.
2 animals/sex/group in Groups 1-4 remained on study, untreated for a 6-day recovery period, and were euthanized on Day 29.
3 animals/sex/group in Group 5 were euthanized on Day 29.

Oral gavage dosing once weekly for three weeks (total of 4 doses) of ONC206 at doses of 1.7, 8.3, and 16.7 mg/kg, or once by intravenous infusion on Day 1 at 8.3 mg/kg, did not have any toxicologic effects on cardiac rhythm or ECG morphology based on the electrocardiograms evaluated during this study.

### Central Nervous System

Functional Observational Battery (CNS) Study Following Repeat Dose Administration (GLP) was performed in rats. The functional observational battery was performed for Groups 1-4 (5 animals/sex/group) prior to treatment initiation and on Days 1, 22 and 28.

TABLE 13
Function observational battery study treatment
groups and corresponding doses

| | Dose | | | | |
|---|---|---|---|---|---|
| | Level* | Concen- | Dose | Number of | |
| | (mg/kg/ | tration* | Volume | Animals** | |
| Group | day) | (mg/mL) | (mL/kg) | Male | Female |
| 1. Control (vehicle) | 0 | 0 | 10 | 15 | 15 |
| 2. ONC206 Low-dose | 5 | 0.5 | 10 | 15 | 15 |
| 3. ONC206 Mid-dose | 25 | 2.5 | 10 | 15 | 15 |
| 4. ONC206 High-dose | 50 | 5.0 | 10 | 15 | 15 |

The dose levels and concentrations shown in the table represent actual API (free base).

A targeted 10 animals/sex/group were euthanized on Day 23. A targeted 5 animals/sex/group remained on study, untreated, for a one week recovery period and were euthanized on Day 29

There were no abnormal observations noted during assessment of the functional observational battery on Days 1, 22 and 28.

### Conclusions and Dose Selection

In general, adverse events associated with the highest weekly oral doses of ONC206 were mild and reversible in GLP toxicology studies. The only findings that were observed in GLP toxicology studies for both rats and dogs were decreased body weight and/or body weight gain with no effects on food consumption. Rats had higher AUC and Cmax concentrations than dogs at all doses and sexes, except for Cmax for high-dose group rat males. Despite the differences in PK the observations were consistent across species. In non-GLP studies, at doses higher than those tested in GLP studies, clinical findings including deaths were observed. Given the safety profile of this drug and the intended indication of advanced cancer patients, the benefit-risk profile of ONC206 warrants evaluation in the proposed first-in-human clinical trial. We utilized the FDA Guidance for Industry: S9 Nonclinical Evaluation for Anticancer Pharmaceuticals (ICH, March 2010) to calculate the proposed starting dose. The starting dose was allometrically converted as 1/10th of the lowest NOAEL (no observed adverse event level) from GLP safety studies in rat and dog. The NOAEL following oral administration of ONC206 once weekly for three weeks to Sprague-Dawley rats in a GLP study is at least 50 mg/kg. The NOAEL following oral administration of ONC206 once weekly for three weeks to Beagle dogs, is at least 16.7 mg/kg. The following calculation was used: 50 mg/kg rat* 1/10*1/6.2 standard conversion factor=0.77 mg/kg. Assuming a 60 kg human adult, this would yield a fixed starting dose of 46.2 mg that is rounded to 50 mg.

### Nonclinical Pharmacokinetics and Metabolism

The absorption, distribution, metabolism, and excretion of ONC206 in humans are unknown.

The pharmacokinetics of ONC206 has been studied in rodents and dogs. Rats and dogs both achieved therapeutic PK based on preclinical efficacy thresholds. Exposure was slightly greater in females for both species. Rats and dogs both had dose dependent and approximately dose proportional exposure. At the top doses tested, rats had approximately 96% and dogs had ˜52% absolute oral bioavailability. Both species had rapid absorption with a plasma terminal half-life that ranged from 0.7 to 7.1 hours. Rats had higher AUC and Cmax concentrations than dogs at all doses and sexes, except for Cmax for rat male/high-dose (Table 14).

TABLE 14
Cmax and AUC values for rats and dogs following first weekly dose of ONC206.
Rat doses for low, mid, and high dose groups are 5, 25, and 50 mg/kg.
Dog doses for low, mid, and high are 1.7, 8.3, and 16.7 mg/kg.

| | | Cmax(ng/mL) | | | | AUC0-inf (ng*hr/mL) | | | |
|---|---|---|---|---|---|---|---|---|---|
| | | Rat | | Dog | | Rat | | Dog | |
| | Dose | Male | Female | Male | Female | Male | Female | Male | Female |
| PO | Low | 161 | 297 | 31.1 | 23.4 | 253 | 1210 | 36.89 | 23.98 |
| | Mid | 961 | 1337 | 374 | 721 | 2980 | 7686 | 477 | 948.5 |
| | High | 919 | 2863 | 2560 | 2304 | 7669 | 14995 | 3762 | 3612 |

No formal metabolic or drug-drug interaction studies with ONC206 or any metabolites have been performed. These studies will be performed later in the development of this agent.

The total exposure to ONC206 does not appear to directly correlate with efficacy in nonclinical studies. This observation is in accordance with delayed and sustained activity of ONC206 observed in vitro or in vivo with infrequent dosing.

Given the relatively short plasma half-life of ONC206, drug accumulation with ONC206 at the weekly dosing schedule is unlikely.

### Effects in Humans

ONC206 has been administered to a single patient in the compassionate use setting. The patient's experience is described below:

Patient UNMC-CUP-01 was a 39-year-old Caucasian male diagnosed with a grade IV H3 K27M-mutant diffuse midline glioma on 2 Dec. 2016. Shortly after diagnosis, the patient received treatment with temozolomide (75 mg/m2/day for 42 consecutive days) and radiation (Total of 60 Gy at 2 Gy per daily fraction). He then received 5 cycles of adjuvant temozolomide (150-200 mg/m2 daily for 5 consecutive days of every 28-day cycle) until radiographic progression was observed on a magnetic resonance image (MRI) of the brain. The patient then began treatment with lomustine at 110 mg/m2 every 6 weeks for 2 cycles. Further radiographic progression was observed on a brain MRI at least 6 weeks after initiation of lomustine. Following treatment with lomustine, the patient received the investigational medication ONC201 for approximately 19 months. In May 2019, the patient was observed to have progressive disease and restarted radiation therapy in July 2019. An MRI done in SEP 2019 showed an increase in T2 intensity in the right thalamus, but decreased contrast enhancement in the right thalamo-capsular area. On 17 Oct. 2019, the treating investigator received approval from the FDA to treat the patient with the investigational molecule ONC206 via a compassionate use protocol. On 14 Nov. 2019, he began treatment with ONC206 at a dose of 50 mg weekly. During treatment the patient experienced adverse events of intermittent confusion (grade 1) and hypophosphatemia (grade 2), which were both considered by the investigator to be possibly related to ONC206. All other adverse events, which were considered by the investigator to be not related to ONC206, included Grade 1 events of dysuria, arthralgia, anxiety, dysphagia, and nausea; Grade 2 events of depression, shortness of breath, muscle weakness, urinary incontinence, and seizure; and a Grade 3 event of nephrolithiasis. On 27 Dec. 2019, the patient took the last dose of ONC206. He discontinued the study on 30 Dec. 2019 and died on 8 Jan. 2020. The death was considered unrelated to ONC206.

### Summary Guidance

### Dosage and Administration

ONC206 investigational drug product is prepared in hydroxypropyl methylcellulose (HPMC) capsules, intended for oral administration. No excipients are used in the drug product. Each capsule of drug product contains the equivalent 50 mg ONC206. ONC206 should be stored, handled and administered in accordance with the parameters as specified in the clinical study protocol.

Patients should take the dose of ONC206 specified by their physician 2 hours prior or 2 hours following food or a meal. If the patient vomits after taking ONC206, they should not retake the dose. Missed doses will not be made up, if more than 3 days from the intended day of administration. ONC206 should be taken with a glass of water and consumed over as short a time as possible. Patients should swallow the capsules as a whole and not chew them. Do not crush or empty the capsule. If vomiting occurs during the course of treatment, no re-dosing of the patient is allowed before the next scheduled dose. The occurrence and frequency of any vomiting during a treatment cycle must be noted as an adverse event.

### Contraindications

ONC206 is contraindicated in patients with known severe (Grade 3 or 4) hypersensitivity reactions to ONC206, its excipients, or related compounds.

Imipridone, small molecule ONC206 is a bitopic DRD2 antagonist and CIpP agonist that exhibits enhanced non-competitive effects, high specificity, nanomolar potency against glioma cells, disruption of DRD2 homodimers and blood brain barrier penetrance.

A first-in-human Phase I trial will be completed using a 3+3 dose escalation and food effect study design evaluating once weekly and more frequent dosing of ONC206 in recurrent and rare primary CNS Neoplasms.

Dose Level 1 and Dose Level 2 have been completed with no DLTs.

An MTD will be established for a phase II trial. Secondary endpoints including response rate and survival outcomes will be determined.

Correlative endpoints evaluating of DRD2, DRD2 dimer, CIpP, DRD5, MYC and MYCN expression will be evaluated during the study.

### ONC-206 Dose Justification Analysis

Individual and mean pharmacokinetic data for ONC206 were generated from available patient samples from completed once weekly dose levels. Mean concentration-time profiles over 72 hours following dosing on Cycle 1, Day 1 for each dose level were superimposed with target thresholds based on efficacious concentrations from Genomics of Drug Sensitivity in Cancer (GDSC) and other human cancer cell line survival studies (among 1088 tumor lines tested, 813 cell lines were sensitive to ONC206 treatment with an average IC₅₀ of 562 nM), as well as IC₅₀ concentrations for DRD2 and CIpP target engagement (FIG. 7). Because of the high distribution of ONC206 into various tissues, including the brain (brain stem:plasma ratio of 2.4), total concentrations of ONC206 in plasma were used for comparisons to target thresholds. As noted, FIG. 9 illustrates mean plasma onc206 concentration-time profiles over 72 hours for ONC-206 once weekly dosing regimens in adult patients with recurrent central nervous system tumors.

Peak plasma concentrations of ONC206 above target thresholds were achieved at 150 mg and 200 mg, but not with 50 and 100 mg weekly doses. Across doses, the peak concentrations were observed with a median Tmax of approximately 1-2 hours and followed by linear elimination with mean t_{1/2} ranging from 11.2 to 17.9 hours.

Clinical safety data for these cohorts indicated that ONC206 is generally well tolerated. Out of 10 patients enrolled in these cohorts, 6 patients experienced a Grade 2 or Grade 3 adverse events considered possibly/probably related to ONC206 by investigator attribution. No adverse events have met dose-limiting toxicity criteria.

In addition to the pharmacokinetic data, additional nonclinical in vitro washout experiments in human glioma cells were completed. These in vitro studies with human brain tumor cell lines showed that sustained exposures to 5-11,000 nanomolar ONC206 concentrations over a period of at least 72 hours are required for optimal efficacy (FIG. 8). An incubation period of 24 hours was ineffective in this cell line. Incubation periods of 36, 48, and 60 hours provided incremental improvements in efficacy with a large increase in maximal response observed with a 72 hour incubation.

Based on these nonclinical data, the plasma concentration profiles of ONC206 twice daily (BID) for 3 consecutive days were projected (FIG. 9). Efficacious concentrations from GDSC and other human cancer cell line survival studies and concentrations for DRD2 and CIpP engagement were again set as target thresholds and superimposed on the projected mean ONC206 concentration profiles.

Based on projections, BID dosing does not result in accumulation and produces similar Cmax to that observed following once weekly dosing. BID dosing also provides greater AUClast than that observed with once weekly dosing. The increase in AUClast is proportional to the number of doses given, with BID dosing for 3 consecutive days producing an AUClast approximately 5.4-fold greater than once weekly dosing. Based on the estimated concentrations, BID dosing results in sustained ONC206 concentrations at target thresholds compared with once weekly dosing, better mimicking the sustained efficacious concentrations in nonclinical cancer cell studies. Still, as observed with once weekly dosing, 50 mg BID and 100 mg BID dosing regimens for 3 consecutive days are not projected to produce ONC206 concentrations that reach target thresholds. Dosing of ONC206 with >150 mg BID for 3 days, is projected to produce sustained ONC206 concentrations above target thresholds.

To confirm these projections, it is planned to study ONC206 in a Cohort (Cohort B) in parallel with the continued escalation with once weekly regimens (Cohort A) to explore dosing with ONC206 BID for 3 consecutive days. Separate patients would be enrolled in each cohort. To ensure safety with a BID dosing regimen, it is planned to initially explore 50 mg ONC206 BID for 3 consecutive days. This dose regimen will result in a total weekly dose of 300 mg ONC206 and has a projected AUClast lower than the highest observed AUClast in the study to date (5270 hr*ng/mL with 200 mg).

### REFERENCES WHICH ARE HEREBY INCORPORATED BY REFERENCE WITH REGARD TO SUCH BACKGROUND TEACHING

Allen, J. E., Crowder, R., and El-Deiry, W. S. (2015). First-In-Class Small Molecule ONC201 Induces DR5 and Cell Death in Tumor but Not Normal Cells to Provide a Wide Therapeutic Index as an Anti-Cancer Agent. PLoS One 10, e0143082.
Allen, J. E., Kline, C. L., Prabhu, V. V., Wagner, J., Ishizawa, J., Madhukar, N., Lev, A., Baumeister, M., Zhou, L., Lulla, A., et al. (2016). Discovery and clinical introduction of first-in-class imipridone ONC201. Oncotarget.
Allen, J. E., Krigsfeld, G., Mayes, P. A., Patel, L., Dicker, D. T., Patel, A. S., Dolloff, N. G., Messaris, E., Scata, K. A., Wang, W., et al. (2013). Dual inactivation of Akt and ERK by TIC10 signals Foxo3a nuclear translocation, TRAIL gene induction, and potent antitumor effects. Sci Transl Med 5, 171ra117.
Arrillaga-Romany, I., Chi, A. S., Allen, J. E., Oster, W., Wen, P. Y., and Batchelor, T. T. (2017). A phase 2 study of the first imipridone ONC201, a selective DRD2 antagonist for oncology, administered every three weeks in recurrent glioblastoma. Oncotarget.
Ishida, C. T., Zhang, Y., Bianchetti, E., Shu, C., Nguyen, T. T., Kleiner, G., Sanchez-Quintero, M., Quinzii, C. M., Westhoff, M. A., Karpel-Massler, G., et al. (2018). Metabolic Reprogramming by Dual AKT/ERK Inhibition Through Imipridones Elicits Unique Vulnerabilities in Glioblastoma. Clin Cancer Res.
Jung, J., Dowdy, T., Tabouret, E., Reynolds, B., Allen, J., Larion, M., Gilbert, M., and Park, D. (2018). ONC206, an imipridone family member, suppresses glioblastoma cells via blocking cancer stemness pathways. Neuro-Oncology 20, vi97.
Lappano, R., and Maggiolini, M. (2011). G protein-coupled receptors: novel targets for drug discovery in cancer. Nat Rev Drug Discov 10, 47-60.
Madhukar, N. S., Khade, P., Huang, L., Gayvert, K., Galletti, G., Stogniew, M., Allen, J. E., Giannakakou, P., and Elemento, O. (2017). A New Big-Data Paradigm For Target Identification And Drug Discovery. bioRxiv.
Prabhu, V. V., Madhukar, N., Wagner, J., Tarapore, R., Garnett, M. J., McDermott, U., Benes, C., Charter, N., Deacon, S., VanEngelenburg, A., et al. (2017). Potent anti-cancer activity of the imipridone ONC206: A selective dopamine D2-like receptor antagonist. In: Proceedings of the 107th Annual Meeting of the American Association for Cancer Research; April 1-5; Washington, DC Philadelphia (PA): AACR; 2017 Abstract nr 4147A.
Stein, M. N., Bertino, J. R., Kaufman, H. L., Mayer, T., Moss, R., Silk, A., Chan, N., Malhotra, J., Rodriguez-Rodriguez, L., Aisner, J., et al. (2017). First-in-human Clinical Trial of Oral ONC201 in Patients with Refractory Solid Tumors. Clin Cancer Res.
Wagner, J., Kline, C. L., Pottorf, R. S., Nallaganchu, B. R., Olson, G. L., Dicker, D. T., Allen, J. E., and El-Deiry, W. S. (2014). The angular structure of ONC201, a TRAIL pathway-inducing compound, determines its potent anti-cancer activity. Oncotarget 5, 12728-12737.
Wagner, J., Kline, C. L., Ralff, M. D., Lev, A., Lulla, A., Zhou, L., Olson, G. L., Nallaganchu, B. R., Benes, C. H., Allen, J. E., et al. (2017). Preclinical evaluation of the imipridone family, analogues of clinical stage anti-cancer small molecule ONC201, reveals potent anti-cancer effects of ONC212.

It will be appreciated by those skilled in the art that changes could be made to the exemplary embodiments shown and described above without departing from the broad inventive concept thereof. To the extent that a described method does not rely on the particular order of steps set forth herein, the particular order of the steps should not be construed as limitation on the claims. The claims directed to a method of the present disclosure should not be limited to the performance of their steps in the order written, and one skilled in the art can readily appreciate that the steps may be varied and still remain within the spirit and scope of the present disclosure.

All references, including publications, patent applications, and patents, cited herein are hereby incorporated by reference to the same extent as if each reference were individually and specifically indicated to be incorporated by reference and were set forth in its entirety herein.

Although this specification contains many specific implementation details, these should not be construed as limitations on the scope of any disclosure or on the scope of what may be claimed, but rather as descriptions of features that may be specific to particular implementations of particular disclosures. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combinations. Particular implementations of the subject matter have been described. Other implementations, alterations, and permutations of the described implementations are within the scope of the following claims as will be apparent to those skilled in the art. For example, the actions recited in the claims may be performed in a different order and still achieve desirable results. Accordingly, the above description of example implementations does not define or constrain this disclosure. Other changes, substitutions, and alterations are also possible without departing from the spirit and scope of this disclosure.

A number of embodiments of the present disclosure have been described. Although this specification contains many specific implementation details, the specific implementation details should not be construed as limitations on the scope of any disclosures or of what may be claimed, but rather as descriptions of features specific to particular embodiments of the present disclosure. Nevertheless, it will be understood that various modifications may be made without departing from the spirit and scope of the claimed disclosure.

1. A method of treating one or more cancer in a subject in need thereof comprising administering ONC-206, 7-benzyl-4-(2,4-difluorobenzyl)-2,4,6,7,8,9-hexahydroimidazo[1,2-a]pyrido[3,4-e]pyrimidin-5(1H)-one, or a salt thereof.

2. The method of claim 1, wherein the cancer is a CNS cancer.

3. The method of claim 1 or 2, wherein the cancer is a brain cancer.

4. The method of any one of claims 1 to 3, wherein the cancer is a glioma.

5. The method of any one of claims 1 to 4, wherein the cancer is selected from the group consisting of one or more of Gliomas, glioneuronal tumors, and neuronal tumors, Adult-type diffuse gliomas, Astrocytoma, IDH-mutant, Oligodendroglioma, IDH-mutant, and 1p/19q-codeleted, Glioblastoma, IDH-wildtype, Pediatric-type diffuse low-grade gliomas, Diffuse astrocytoma, MYB- or MYBL1-altered, Angiocentric glioma, Polymorphous low-grade neuroepithelial tumor of the young, Diffuse low-grade glioma, MAPK pathway-altered, Pediatric-type diffuse high-grade gliomas, Diffuse midline glioma, H3 K27-altered, Diffuse hemispheric glioma, H3 G34-mutant, Diffuse pediatric-type high-grade glioma, H3-wildtype and IDH-wildtype, Infant-type hemispheric glioma, Circumscribed astrocytic glioma, Pilocytic astrocytoma, High-grade astrocytoma with piloid features, Pleomorphic xanthoastrocytoma, Subependymal giant cell astrocytoma, Chordoid glioma, Astroblastoma, MN1-altered, Glioneuronal and neuronal tumors, Ganglioglioma, Desmoplastic infantile ganglioglioma/desmoplastic infantile astrocytoma, Dysembryoplastic neuroepithelial tumor, Diffuse glioneuronal tumor with oligodendroglioma-like features and nuclear clusters, Papillary glioneuronal tumor, Rosette-forming glioneuronal tumor, Myxoid glioneuronal tumor, Diffuse leptomeningeal glioneuronal tumor, Gangliocytoma, Multinodular and vacuolating neuronal tumor, Dysplastic cerebellar gangliocytoma (Lhermitte-Duclos disease), Central neurocytoma, Extraventricular neurocytoma, Cerebellar liponeurocytoma, Ependymal tumors, Supratentorial ependymoma, Supratentorial ependymoma, ZFTA fusion-positive, Supratentorial ependymoma, YAP1 fusion-positive, Posterior fossa ependymoma, Posterior fossa ependymoma, group PFA, Posterior fossa ependymoma, group PFB, Spinal ependymoma, Spinal ependymoma, MYCN-amplified, Myxopapillary ependymoma, Subependymoma, Choroid plexus tumors, Choroid plexus papilloma, Atypical choroid plexus papilloma, Choroid plexus carcinoma, Embryonal tumors, Medulloblastoma, Medulloblastomas, molecularly defined, Medulloblastoma, WNT-activated, Medulloblastoma, SHH-activated and TP53-wildtype, Medulloblastoma, SHH-activated and TP53-mutant, Medulloblastoma, non-WNT/non-SHH, Medulloblastomas, histologically defined, Other CNS embryonal tumors, Atypical teratoid/rhabdoid tumor, Cribriform neuroepithelial tumor, Embryonal tumor with multilayered rosettes, CNS neuroblastoma, FOXR2-activated, CNS tumor with BCOR internal tandem duplication, CNS embryonal tumor, Pineal tumors, Pineocytoma, Pineal parenchymal tumor of intermediate differentiation, Pineoblastoma, Papillary tumor of the pineal region, Desmoplastic myxoid tumor of the pineal region, SMARCB1-mutant, Cranial and paraspinal nerve tumors, Schwannoma, Neurofibroma, Perineurioma, Hybrid nerve sheath tumor, Malignant melanotic nerve sheath tumor, Malignant peripheral nerve sheath tumor, Paraganglioma, Meningiomas, Meningioma, Mesenchymal, non-meningothelial tumors, Soft tissue tumors, Fibroblastic and myofibroblastic tumors, Solitary fibrous tumor, Vascular tumors, Hemangiomas and vascular malformations, Hemangioblastoma, Skeletal muscle tumors, Rhabdomyosarcoma, Uncertain differentiation, Intracranial mesenchymal tumor, FET-CREB fusion-positive, CIC-rearranged sarcoma, Primary intracranial sarcoma, DICER1-mutant, Ewing sarcoma, Chondro-osseous tumors, Chondrogenic tumors, Mesenchymal chondrosarcoma, Chondrosarcoma, Notochordal tumors, Chordoma (including poorly differentiated chordoma), Melanocytic tumors, Diffuse meningeal melanocytic neoplasms, Meningeal melanocytosis and meningeal melanomatosis, Circumscribed meningeal melanocytic neoplasms, Meningeal melanocytoma and meningeal melanoma, Hematolymphoid tumors, Lymphomas, CNS lymphomas, Primary diffuse large B-cell lymphoma of the CNS, Immunodeficiency-associated CNS lymphoma, Lymphomatoid granulomatosis, Intravascular large B-cell lymphoma, Miscellaneous rare lymphomas in the CNS, MALT lymphoma of the dura, Other low-grade B-cell lymphomas of the CNS, Anaplastic large cell lymphoma (ALK+/ALK−), T-cell and NK/T-cell lymphomas, Histiocytic tumors, Erdheim-Chester disease, Rosai-Dorfman disease, Juvenile xanthogranuloma, Langerhans cell histiocytosis, Histiocytic sarcoma, Germ cell tumors, Mature teratoma, Immature teratoma, Teratoma with somatic-type malignancy, Germinoma, Embryonal carcinoma, Yolk sac tumor, Choriocarcinoma, Mixed germ cell tumor, Tumors of the sellar region, Adamantinomatous craniopharyngioma, Papillary craniopharyngioma, Pituicytoma, granular cell tumor of the sellar region, and spindle cell oncocytoma, Pituitary adenoma/PitNET, Pituitary blastoma, Metastases to the CNS, Metastases to the brain and spinal cord parenchyma, and Metastases to the meninges.

6. The method of any one of claims 1 to 5, wherein the cancer is one or more of Pilocytic astrocytomas, Diffuse astrocytomas, Anaplastic astrocytomas, Glioblastomas, Oligodendroglial tumors, Ependymal tumor, Medulloblastomas, Pineal tumors, Meningeal tumors, and Germ cell tumors

7. The method of any one of claims 1 to 6, wherein the administration reduces one or more symptom of the cancer.

8. The method of any one of claims 1 to 7, wherein the administration reduces tumor growth.

9. The method of any one of claims 1 to 8, wherein the administration provides one or more of (i) reduction in tumor size, (ii) progression-free survival, (iii) overall survival, (iv) patient-reported outcomes, (v) disease-free survival, (vi) objective response, (vii) complete response, (viii) increased time to progression, (ix) reduced corticosteroid use, (x) reduced supportive medication, (xi) reduced incidence of seizures, (xii) reduced use of anti seizure medication, (xiii) increased qualify of life, (xiv) reduced neurological deficits, and (xv) other objective response

10. The method of any one of claims 1 to 9, wherein the administration is made in combination with one or more additional therapy or therapeutic agent.

11. The method of any one of claims 1 to 10, wherein the dose of ONC-206 or a salt thereof is from about 25 mg to about 75 mg, based on free base form.

12. The method of claim 11, wherein the dose is about 50 mg.

13. The method of any one of claims 1 to 12, wherein the starting or loading dose of ONC-206 or a salt thereof is from about 5 mg/kg to about 100 mg/kg.

14. The method of claim 13, wherein the maintenance dose is about 50 mg/kg.

15. The method of any one of claims 1 to 10, wherein the dose of ONC-206 or a salt thereof at a volume of 10 ml/kg is from about 0.6 mg/l to about 10 mg/ml.

16. The method of claim 15, wherein the dose is about 5 mg/ml.

17. The method of any one of claims 1 to 10, wherein the dose of ONC-206 or a salt thereof is from about 12.5 mg/kg/day to about 25 mg/kg/day.

18. The method of claim 17, wherein the dose is about 12.5 mg/kg/day.

19. The method of any one of claims 1 to 10, wherein the dose of ONC-206 or a salt thereof is from about 8 mg/kg to about 20 mg/kg.

20. The method of any one of claims 1 to 10, wherein the dose of ONC-206 or a salt thereof is from about 5 mg/kg/day to about 50 mg/kg/day.

21. The method of claim 20, wherein the dose is less than about 50 mg/kg/day.

22. The method of any one of claims 1 to 10, wherein the dose of ONC-206 or a salt thereof is from about 1.7 mg/kg/day to about 16.7 mg/kg/day.

23. The method of claim 22, wherein the dose is greater than about 16.7 mg/kg/day.

24. The method of any one of claims 1 to 10, wherein the dose provided is daily, twice a week, three times a week, four times a week, five times a week, six times a week, weekly, bi-weekly, or monthly.

25. The method of claim 24, wherein the dose is three times a day, twice daily, daily, every other day, every third day, every fourth day, every fifth day, every sixth day, or weekly.

26. The method of any one of claims 1 to 25, wherein the Cmax is from about 4 μM to about 20 μM.

27. The method of claim 26, wherein the terminal half-life is about 6 hours.

28. The method of any one of claims 1 to 27, wherein a target tissue distribution relative to plasma ONC-206 concentation is at least one or more of 2 fold, 3 fold, 4 fold, 5 fold, 6 fold, 7 fold, 8 fold, 9 fold, or 10 fold higher.

29. The method of any one of claims 1 to 28, wherein the ONC-206 is provided as the di-HCl salt.

30. The method of any one of claims 1 to 29, wherein the treatment relates to a recurrent neoplasm.

31. The method of any one of claims 1 to 29, wherein the treatment is other than a first line treatment.

32. The method of any one of claims 1 to 29, wherein the treatment is to an advanced cancer.

33. The method of any one of claims 1 to 29, wherein the treatment is administered at least 30 days post-radiation.

34. The method of any one of claims 1 to 29, wherein the treatment is administered at least 60 days post-radiation.

35. The method of any one of claims 1 to 29, wherein the treatment is administered at least 90 days post-radiation.

36. The method of any one of claims 1 to 29, wherein the treatment is administered after surgical resection.

37. The method of any one of claims 1 to 36, wherein the CNS neoplasm is one or more of recurrent glioblastoma, WHO Grade 1, 2, 3, or 4 CNS tumor types, infiltrating glialneoplasms, DMG H3K27M, DMG H3 K27-altered, DMG H3 K27me-loss (H3K27me3), ependymoma, medulloblastoma, malignantmeningiomas, and other rare primary CNS neoplasms.

38. The method of any one of claims 1 to 37, wherein the administration is monitored with one or more of DRD2, DRD2 dimer, CIpP, CIpP substrates SDHA, SDHB, and markers of oxidative phosphorylation, DRD5, c-myc, and n-myc expression.

39. The method of any one of claims 1 to 38, wherein objective response rate is measured by one or more of RANO criteria, overall survical, progression-free survival, and disease control rate.

40. Use of ONC-206, 7-benzyl-4-(2,4-difluorobenzyl)-2,4,6,7,8,9-hexahydroimidazo[1,2-a]pyrido[3,4-e]pyrimidin-5(1H)-one, or a salt thereof, in the preparation of a medicament for treating one or more cancer.

41. The use of claim 40, wherein the cancer is a CNS cancer.

42. The use of claim 40 or 41, wherein the cancer is a brain cancer.

43. The use of any one of claims 40 to 42, wherein the cancer is a glioma.

44. The use of any one of claims 40 to 43, wherein the cancer is selected from the group consisting of one or more of Gliomas, glioneuronal tumors, and neuronal tumors, Adult-type diffuse gliomas, Astrocytoma, IDH-mutant, Oligodendroglioma, IDH-mutant, and 1p/19q-codeleted, Glioblastoma, IDH-wildtype, Pediatric-type diffuse low-grade gliomas, Diffuse astrocytoma, MYB- or MYBL1-altered, Angiocentric glioma, Polymorphous low-grade neuroepithelial tumor of the young, Diffuse low-grade glioma, MAPK pathway-altered, Pediatric-type diffuse high-grade gliomas, Diffuse midline glioma, H3 K27-altered, Diffuse hemispheric glioma, H3 G34-mutant, Diffuse pediatric-type high-grade glioma, H3-wildtype and IDH-wildtype, Infant-type hemispheric glioma, Circumscribed astrocytic glioma, Pilocytic astrocytoma, High-grade astrocytoma with piloid features, Pleomorphic xanthoastrocytoma, Subependymal giant cell astrocytoma, Chordoid glioma, Astroblastoma, MN1-altered, Glioneuronal and neuronal tumors, Ganglioglioma, Desmoplastic infantile ganglioglioma/desmoplastic infantile astrocytoma, Dysembryoplastic neuroepithelial tumor, Diffuse glioneuronal tumor with oligodendroglioma-like features and nuclear clusters, Papillary glioneuronal tumor, Rosette-forming glioneuronal tumor, Myxoid glioneuronal tumor, Diffuse leptomeningeal glioneuronal tumor, Gangliocytoma, Multinodular and vacuolating neuronal tumor, Dysplastic cerebellar gangliocytoma (Lhermitte-Duclos disease), Central neurocytoma, Extraventricular neurocytoma, Cerebellar liponeurocytoma, Ependymal tumors, Supratentorial ependymoma, Supratentorial ependymoma, ZFTA fusion-positive, Supratentorial ependymoma, YAP1 fusion-positive, Posterior fossa ependymoma, Posterior fossa ependymoma, group PFA, Posterior fossa ependymoma, group PFB, Spinal ependymoma, Spinal ependymoma, MYCN-amplified, Myxopapillary ependymoma, Subependymoma, Choroid plexus tumors, Choroid plexus papilloma, Atypical choroid plexus papilloma, Choroid plexus carcinoma, Embryonal tumors, Medulloblastoma, Medulloblastomas, molecularly defined, Medulloblastoma, WNT-activated, Medulloblastoma, SHH-activated and TP53-wildtype, Medulloblastoma, SHH-activated and TP53-mutant, Medulloblastoma, non-WNT/non-SHH, Medulloblastomas, histologically defined, Other CNS embryonal tumors, Atypical teratoid/rhabdoid tumor, Cribriform neuroepithelial tumor, Embryonal tumor with multilayered rosettes, CNS neuroblastoma, FOXR2-activated, CNS tumor with BCOR internal tandem duplication, CNS embryonal tumor, Pineal tumors, Pineocytoma, Pineal parenchymal tumor of intermediate differentiation, Pineoblastoma, Papillary tumor of the pineal region, Desmoplastic myxoid tumor of the pineal region, SMARCB1-mutant, Cranial and paraspinal nerve tumors, Schwannoma, Neurofibroma, Perineurioma, Hybrid nerve sheath tumor, Malignant melanotic nerve sheath tumor, Malignant peripheral nerve sheath tumor, Paraganglioma, Meningiomas, Meningioma, Mesenchymal, non-meningothelial tumors, Soft tissue tumors, Fibroblastic and myofibroblastic tumors, Solitary fibrous tumor, Vascular tumors, Hemangiomas and vascular malformations, Hemangioblastoma, Skeletal muscle tumors, Rhabdomyosarcoma, Uncertain differentiation, Intracranial mesenchymal tumor, FET-CREB fusion-positive, CIC-rearranged sarcoma, Primary intracranial sarcoma, DICER1-mutant, Ewing sarcoma, Chondro-osseous tumors, Chondrogenic tumors, Mesenchymal chondrosarcoma, Chondrosarcoma, Notochordal tumors, Chordoma (including poorly differentiated chordoma), Melanocytic tumors, Diffuse meningeal melanocytic neoplasms, Meningeal melanocytosis and meningeal melanomatosis, Circumscribed meningeal melanocytic neoplasms, Meningeal melanocytoma and meningeal melanoma, Hematolymphoid tumors, Lymphomas, CNS lymphomas, Primary diffuse large B-cell lymphoma of the CNS, Immunodeficiency-associated CNS lymphoma, Lymphomatoid granulomatosis, Intravascular large B-cell lymphoma, Miscellaneous rare lymphomas in the CNS, MALT lymphoma of the dura, Other low-grade B-cell lymphomas of the CNS, Anaplastic large cell lymphoma (ALK+/ALK−), T-cell and NK/T-cell lymphomas, Histiocytic tumors, Erdheim-Chester disease, Rosai-Dorfman disease, Juvenile xanthogranuloma, Langerhans cell histiocytosis, Histiocytic sarcoma, Germ cell tumors, Mature teratoma, Immature teratoma, Teratoma with somatic-type malignancy, Germinoma, Embryonal carcinoma, Yolk sac tumor, Choriocarcinoma, Mixed germ cell tumor, Tumors of the sellar region, Adamantinomatous craniopharyngioma, Papillary craniopharyngioma, Pituicytoma, granular cell tumor of the sellar region, and spindle cell oncocytoma, Pituitary adenoma/PitNET, Pituitary blastoma, Metastases to the CNS, Metastases to the brain and spinal cord parenchyma, and Metastases to the meninges.

45. The use of claim 40, wherein the cancer is one or more of Pilocytic astrocytomas, Diffuse astrocytomas, Anaplastic astrocytomas, Glioblastomas, Oligodendroglial tumors, Ependymal tumor, Medulloblastomas, Pineal tumors, Meningeal tumors, and Germ cell tumors

46. The use of any one of claims 40 to 45, wherein the administration reduces one or more symptom of the cancer.

47. The use of any one of claims 40 to 46, wherein the administration reduces tumor growth.

48. The use of any one of claims 40 to 47, wherein the administration provides one or more of (i) reduction in tumor size, (ii) progression-free survival, (iii) overall survival, (iv) patient-reported outcomes, (v) disease-free survival, (vi) objective response, (vii) complete response, (viii) increased time to progression, (ix) reduced corticosteroid use, (x) reduced supportive medication, (xi) reduced incidence of seizures, (xii) reduced use of anti seizure medication, (xiii) increased qualify of life, (xiv) reduced neurological deficits, and (xv) other objective response.

49. The use of any one of claims 40 to 48, wherein the administration is made in combination with one or more additional therapy or therapeutic agent.

50. The use of any one of claims 40 to 49, wherein the dose of ONC-206 or a salt thereof is from about 25 mg to about 75 mg, based on free base form.

51. The use of claim 50, wherein the dose is about 50 mg.

52. The use of any one of claims 40 to 51, wherein the dose of ONC-206 or a salt thereof is from about 5 mg/kg to about 1000 mg/kg.

53. The use of claim 52, wherein the dose is about 10 mg/kg, 15 mg/kg, 20 mg/kg, 25 mg/kg, 30 mg/kg, 35 mg/kg, 40 mg/kg, 45 mg/kg, or 50 mg/kg.

54. The use of any one of claims 40 to 53, wherein the dose of ONC-206 or a salt thereof at a volume of 10 ml/kg is from about 0.6 mg/l to about 10 mg/ml.

55. The use of claim 54, wherein the dose is about 5 mg/ml.

56. The use of any one of claims 40 to 55, wherein the dose of ONC-206 or a salt thereof is from about 12.5 mg/kg/day to about 25 mg/kg/day.

57. The use of claim 56, wherein the dose is about 12.5 mg/kg/day.

58. The use of any one of claims 40 to 57, wherein the dose of ONC-206 or a salt thereof is from about 8 mg/kg to about 20 mg/kg.

59. The use of any one of claims 40 to 58, wherein the dose of ONC-206 or a salt thereof is from about 5 mg/kg/day to about 50 mg/kg/day.

60. The use of claim 59, wherein the dose is less than about 50 mg/kg/day.

61. The use of any one of claims 40 to 60, wherein the dose of ONC-206 or a salt thereof is from about 1.7 mg/kg/day to about 16.7 mg/kg/day.

62. The use of claim 61, wherein the dose is greater than about 16.7 mg/kg/day.

63. The use of any one of claims 40 to 62, wherein the dose provided is daily, twice a week, three times a week, four times a week, five times a week, six times a week, weekly, bi-weekly, or monthly.

64. The use of claim 63, wherein the dose is three times a day, twice daily, daily, every other day, every third day, every fourth day, every fifth day, every sixth day, or weekly.

65. The use of any one of claims 40 to 64, wherein the Cmax is from about 4 μM to about 20 μM.

66. The use of claim 65, wherein the terminal half-life is about 6 hours.

67. The use of any one of claims 40 to 66, wherein a target tissue distribution relative to plasma ONC-206 concentation is at least one or more of 2 fold, 3 fold, 4 fold, 5 fold, 6 fold, 7 fold, 8 fold, 9 fold, or 10 fold higher.

68. The use of any one of claims 40 to 67, wherein the ONC-206 is provided as the di-HCl salt.

69. The use of any one of claims 40 to 68, wherein the treatment relates to a recurrent neoplasm.

70. The use of any one of claims 40 to 69, wherein the treatment is other than a first line treatment.

71. The use of any one of claims 40 to 68, wherein the treatment is to an advanced cancer.

72. The use of any one of claims 40 to 68, wherein the treatment is administered at least 30 days post-radiation.

73. The use of any one of claims 40 to 68, wherein the treatment is administered at least 60 days post-radiation.

74. The use of any one of claims 40 to 68, wherein the treatment is administered at least 90 days post-radiation.

75. The use of any one of claims 40 to 68, wherein the treatment is administered after surgical resection.

76. The use of any one of claims 40 to 75, wherein the CNS neoplasm is one or more of recurrent glioblastoma, WHO Grade 1, 2, 3, or 4 CNS tumor types, infiltrating glialneoplasms, DMG H3K27M, DMG H3 K27-altered, DMG H3 K27me-loss (H3K27me3), ependymoma, medulloblastoma, malignantmeningiomas, and other rare primary CNS neoplasms.

77. The use of any one of claims 40 to 76, wherein the administration is monitored with one or more of DRD2, DRD2 dimer, CIpP, CIpP substrates SDHA, SDHB, and markers of oxidative phosphorylation, DRD5, c-myc, and n-myc expression.

78. The use of any one of claims 40 to 77, wherein objective response rate is measured by one or more of RANO criteria, overall survival, progression-free survival, and disease control rate.

79. A compound ONC-206, 7-benzyl-4-(2,4-difluorobenzyl)-2,4,6,7,8,9-hexahydroimidazo[1,2-a]pyrido[3,4-e]pyrimidin-5(1H)-one, or a salt thereof, for use in the preparation of a medicament for treating one or more cancer

80. The compound of claim 79, wherein the cancer is a CNS cancer.

81. The compound of claim 79 or 80, wherein the cancer is a brain cancer.

82. The compound of any one of claims 79 to 81, wherein the cancer is a glioma.

83. The compound of any one of claims 79 to 82, wherein the cancer is selected from the group consisting of one or more of Gliomas, glioneuronal tumors, and neuronal tumors, Adult-type diffuse gliomas, Astrocytoma, IDH-mutant, Oligodendroglioma, IDH-mutant, and 1p/19q-codeleted, Glioblastoma, IDH-wildtype, Pediatric-type diffuse low-grade gliomas, Diffuse astrocytoma, MYB- or MYBL1-altered, Angiocentric glioma, Polymorphous low-grade neuroepithelial tumor of the young, Diffuse low-grade glioma, MAPK pathway-altered, Pediatric-type diffuse high-grade gliomas, Diffuse midline glioma, H3 K27-altered, Diffuse hemispheric glioma, H3 G34-mutant, Diffuse pediatric-type high-grade glioma, H3-wildtype and IDH-wildtype, Infant-type hemispheric glioma, Circumscribed astrocytic glioma, Pilocytic astrocytoma, High-grade astrocytoma with piloid features, Pleomorphic xanthoastrocytoma, Subependymal giant cell astrocytoma, Chordoid glioma, Astroblastoma, MN1-altered, Glioneuronal and neuronal tumors, Ganglioglioma, Desmoplastic infantile ganglioglioma/desmoplastic infantile astrocytoma, Dysembryoplastic neuroepithelial tumor, Diffuse glioneuronal tumor with oligodendroglioma-like features and nuclear clusters, Papillary glioneuronal tumor, Rosette-forming glioneuronal tumor, Myxoid glioneuronal tumor, Diffuse leptomeningeal glioneuronal tumor, Gangliocytoma, Multinodular and vacuolating neuronal tumor, Dysplastic cerebellar gangliocytoma (Lhermitte-Duclos disease), Central neurocytoma, Extraventricular neurocytoma, Cerebellar liponeurocytoma, Ependymal tumors, Supratentorial ependymoma, Supratentorial ependymoma, ZFTA fusion-positive, Supratentorial ependymoma, YAP1 fusion-positive, Posterior fossa ependymoma, Posterior fossa ependymoma, group PFA, Posterior fossa ependymoma, group PFB, Spinal ependymoma, Spinal ependymoma, MYCN-amplified, Myxopapillary ependymoma, Subependymoma, Choroid plexus tumors, Choroid plexus papilloma, Atypical choroid plexus papilloma, Choroid plexus carcinoma, Embryonal tumors, Medulloblastoma, Medulloblastomas, molecularly defined, Medulloblastoma, WNT-activated, Medulloblastoma, SHH-activated and TP53-wildtype, Medulloblastoma, SHH-activated and TP53-mutant, Medulloblastoma, non-WNT/non-SHH, Medulloblastomas, histologically defined, Other CNS embryonal tumors, Atypical teratoid/rhabdoid tumor, Cribriform neuroepithelial tumor, Embryonal tumor with multilayered rosettes, CNS neuroblastoma, FOXR2-activated, CNS tumor with BCOR internal tandem duplication, CNS embryonal tumor, Pineal tumors, Pineocytoma, Pineal parenchymal tumor of intermediate differentiation, Pineoblastoma, Papillary tumor of the pineal region, Desmoplastic myxoid tumor of the pineal region, SMARCB1-mutant, Cranial and paraspinal nerve tumors, Schwannoma, Neurofibroma, Perineurioma, Hybrid nerve sheath tumor, Malignant melanotic nerve sheath tumor, Malignant peripheral nerve sheath tumor, Paraganglioma, Meningiomas, Meningioma, Mesenchymal, non-meningothelial tumors, Soft tissue tumors, Fibroblastic and myofibroblastic tumors, Solitary fibrous tumor, Vascular tumors, Hemangiomas and vascular malformations, Hemangioblastoma, Skeletal muscle tumors, Rhabdomyosarcoma, Uncertain differentiation, Intracranial mesenchymal tumor, FET-CREB fusion-positive, CIC-rearranged sarcoma, Primary intracranial sarcoma, DICER1-mutant, Ewing sarcoma, Chondro-osseous tumors, Chondrogenic tumors, Mesenchymal chondrosarcoma, Chondrosarcoma, Notochordal tumors, Chordoma (including poorly differentiated chordoma), Melanocytic tumors, Diffuse meningeal melanocytic neoplasms, Meningeal melanocytosis and meningeal melanomatosis, Circumscribed meningeal melanocytic neoplasms, Meningeal melanocytoma and meningeal melanoma, Hematolymphoid tumors, Lymphomas, CNS lymphomas, Primary diffuse large B-cell lymphoma of the CNS, Immunodeficiency-associated CNS lymphoma, Lymphomatoid granulomatosis, Intravascular large B-cell lymphoma, Miscellaneous rare lymphomas in the CNS, MALT lymphoma of the dura, Other low-grade B-cell lymphomas of the CNS, Anaplastic large cell lymphoma (ALK+/ALK−), T-cell and NK/T-cell lymphomas, Histiocytic tumors, Erdheim-Chester disease, Rosai-Dorfman disease, Juvenile xanthogranuloma, Langerhans cell histiocytosis, Histiocytic sarcoma, Germ cell tumors, Mature teratoma, Immature teratoma, Teratoma with somatic-type malignancy, Germinoma, Embryonal carcinoma, Yolk sac tumor, Choriocarcinoma, Mixed germ cell tumor, Tumors of the sellar region, Adamantinomatous craniopharyngioma, Papillary craniopharyngioma, Pituicytoma, granular cell tumor of the sellar region, and spindle cell oncocytoma, Pituitary adenoma/PitNET, Pituitary blastoma, Metastases to the CNS, Metastases to the brain and spinal cord parenchyma, and Metastases to the meninges.

84. The compound of claim 79, wherein the cancer is one or more of Pilocytic astrocytomas, Diffuse astrocytomas, Anaplastic astrocytomas, Glioblastomas, Oligodendroglial tumors, Ependymal tumor, Medulloblastomas, Pineal tumors, Meningeal tumors, and Germ cell tumors

85. The compound of any one of claims 79 to 84, wherein the administration reduces one or more symptom of the cancer.

86. The compound of any one of claims 79 to 85, wherein the administration reduces tumor growth.

87. The compound of any one of claims 79 to 86, wherein the administration provides one or more of (i) reduction in tumor size, (ii) progression-free survival, (iii) overall survival, (iv) patient-reported outcomes, (v) disease-free survival, (vi) objective response, (vii) complete response, (viii) increased time to progression, (ix) reduced corticosteroid use, (x) reduced supportive medication, (xi) reduced incidence of seizures, (xii) reduced use of anti seizure medication, (xiii) increased qualify of life, (xiv) reduced neurological deficits, and (xv) other objective response.

88. The compound of any one of claims 79 to 87, wherein the administration is made in combination with one or more additional therapy or therapeutic agent.

89. The compound of any one of claims 79 to 88, wherein the dose of ONC-206 or a salt thereof is from about 25 mg to about 75 mg, based on free base form.

90. The use of claim 89, wherein the dose is about 50 mg.

91. The compound of any one of claims 79 to 88, wherein the dose of ONC-206 or a salt thereof is from about 5 mg/kg to about 1000 mg/kg.

92. The use of claim 91, wherein the dose is about 10 mg/kg, 15 mg/kg, 20 mg/kg, 25 mg/kg, 30 mg/kg, 35 mg/kg, 40 mg/kg, 45 mg/kg, or 50 mg/kg.

93. The compound of any one of claims 79 to 88, wherein the dose of ONC-206 or a salt thereof at a volume of 10 ml/kg is from about 0.6 mg/l to about 10 mg/ml.

94. The use of claim 93, wherein the dose is about 5 mg/ml.

95. The compound of any one of claims 79 to 88, wherein the dose of ONC-206 or a salt thereof is from about 12.5 mg/kg/day to about 25 mg/kg/day.

96. The use of claim 95, wherein the dose is about 12.5 mg/kg/day.

97. The compound of any one of claims 79 to 88, wherein the dose of ONC-206 or a salt thereof is from about 8 mg/kg to about 20 mg/kg.

98. The compound of any one of claims 79 to 88, wherein the dose of ONC-206 or a salt thereof is from about 5 mg/kg/day to about 50 mg/kg/day.

99. The use of claim 98, wherein the dose is less than about 50 mg/kg/day.

100. The compound of any one of claims 79 to 88, wherein the dose of ONC-206 or a salt thereof is from about 1.7 mg/kg/day to about 16.7 mg/kg/day.

101. The use of claim 100, wherein the dose is greater than about 16.7 mg/kg/day.

102. The compound of any one of claims 79 to 101, wherein the dose provided is daily, twice a week, three times a week, four times a week, five times a week, six times a week, weekly, bi-weekly, or monthly.

103. The use of claim 102, wherein the dose is three times a day, twice daily, daily, every other day, every third day, every fourth day, every fifth day, every sixth day, or weekly.

104. The compound of any one of claims 79 to 103, wherein the Cmax is from about 4 μM to about 20 μM.

105. The use of claim 104, wherein the terminal half-life is about 6 hours.

106. The compound of any one of claims 79 to 105, wherein a target tissue distribution relative to plasma ONC-206 concentation is at least one or more of 2 fold, 3 fold, 4 fold, 5 fold, 6 fold, 7 fold, 8 fold, 9 fold, or 10 fold higher.

107. The compound of any one of claims 79 to 106, wherein the ONC-206 is provided as the di-HCl salt.

108. The compound of any one of claims 79 to 107, wherein the treatment relates to a recurrent neoplasm.

109. The compound of any one of claims 79 to 107, wherein the treatment is other than a first line treatment.

110. The compound of any one of claims 79 to 107, wherein the treatment is to an advanced cancer.

111. The compound of any one of claims 79 to 107, wherein the treatment is administered at least 30 days post-radiation.

112. The compound of any one of claims 79 to 107, wherein the treatment is administered at least 60 days post-radiation.

113. The compound of any one of claims 79 to 107, wherein the treatment is administered at least 90 days post-radiation.

114. The compound of any one of claims 79 to 107, wherein the treatment is administered after surgical resection.

115. The compound of any one of claims 79 to 114, wherein the CNS neoplasm is one or more of recurrent glioblastoma, WHO Grade 2 and 3 infiltrating glialneoplasms, DMG H3K27M, DMG H3 K27-altered, DMG H3 K27me-loss (H3K27me3), ependymoma, medulloblastoma, malignant meningiomas, and other rare primary CNS neoplasms.

116. The compound of any one of claims 79 to 115, wherein the administration is monitored with one or more of DRD2, DRD2 dimer, CIpP, CIpP substrates SDHA, SDHB, and markers of oxidative phosphorylation, DRD5, c-myc, and n-myc expression.

117. The compound of any one of claims 79 to 116, wherein objective response rate is measured by one or more of RANO criteria, overall survival, progression-free survival, and disease control rate.

118. A method, use, or compound for use for treating one or more cancer in a subject in need thereof comprising administering ONC-206: 7-benzyl-4-(2,4-difluorobenzyl)-2,4,6,7,8,9-hexahydroimidazo[1,2-a]pyrido[3,4-e]pyrimidin-5(1H)-one, or a salt thereof, at a dose of about 50 mg twice daily for three consecutive days.

119. The method, use, or compound for use of claim 118, wherein the administering for three consecutive days is followed by four days without dosing ONC-206.

120. The method, use, or compound for use of either claim 118 or 119, wherein the cancer is a CNS cancer.

121. The method, use, or compound for use of claim 118 to 120, wherein the cancer is a brain cancer.

122. The method, use, or compound for use of any one of claims 118 to 121, wherein the cancer is a glioma.

123. The method, use, or compound for use of any one of claims 118 to 122, wherein the cancer is selected from the group consisting of one or more of Gliomas, glioneuronal tumors, and neuronal tumors, Adult-type diffuse gliomas, Astrocytoma, IDH-mutant, Oligodendroglioma, IDH-mutant, and 1p/19q-codeleted, Glioblastoma, IDH-wildtype, Pediatric-type diffuse low-grade gliomas, Diffuse astrocytoma, MYB- or MYBL1-altered, Angiocentric glioma, Polymorphous low-grade neuroepithelial tumor of the young, Diffuse low-grade glioma, MAPK pathway-altered, Pediatric-type diffuse high-grade gliomas, Diffuse midline glioma, H3 K27-altered, Diffuse hemispheric glioma, H3 G34-mutant, Diffuse pediatric-type high-grade glioma, H3-wildtype and IDH-wildtype, Infant-type hemispheric glioma, Circumscribed astrocytic glioma, Pilocytic astrocytoma, High-grade astrocytoma with piloid features, Pleomorphic xanthoastrocytoma, Subependymal giant cell astrocytoma, Chordoid glioma, Astroblastoma, MN1-altered, Glioneuronal and neuronal tumors, Ganglioglioma, Desmoplastic infantile ganglioglioma/desmoplastic infantile astrocytoma, Dysembryoplastic neuroepithelial tumor, Diffuse glioneuronal tumor with oligodendroglioma-like features and nuclear clusters, Papillary glioneuronal tumor, Rosette-forming glioneuronal tumor, Myxoid glioneuronal tumor, Diffuse leptomeningeal glioneuronal tumor, Gangliocytoma, Multinodular and vacuolating neuronal tumor, Dysplastic cerebellar gangliocytoma (Lhermitte-Duclos disease), Central neurocytoma, Extraventricular neurocytoma, Cerebellar liponeurocytoma, Ependymal tumors, Supratentorial ependymoma, Supratentorial ependymoma, ZFTA fusion-positive, Supratentorial ependymoma, YAP1 fusion-positive, Posterior fossa ependymoma, Posterior fossa ependymoma, group PFA, Posterior fossa ependymoma, group PFB, Spinal ependymoma, Spinal ependymoma, MYCN-amplified, Myxopapillary ependymoma, Subependymoma, Choroid plexus tumors, Choroid plexus papilloma, Atypical choroid plexus papilloma, Choroid plexus carcinoma, Embryonal tumors, Medulloblastoma, Medulloblastomas, molecularly defined, Medulloblastoma, WNT-activated, Medulloblastoma, SHH-activated and TP53-wildtype, Medulloblastoma, SHH-activated and TP53-mutant, Medulloblastoma, non-WNT/non-SHH, Medulloblastomas, histologically defined, Other CNS embryonal tumors, Atypical teratoid/rhabdoid tumor, Cribriform neuroepithelial tumor, Embryonal tumor with multilayered rosettes, CNS neuroblastoma, FOXR2-activated, CNS tumor with BCOR internal tandem duplication, CNS embryonal tumor, Pineal tumors, Pineocytoma, Pineal parenchymal tumor of intermediate differentiation, Pineoblastoma, Papillary tumor of the pineal region, Desmoplastic myxoid tumor of the pineal region, SMARCB1-mutant, Cranial and paraspinal nerve tumors, Schwannoma, Neurofibroma, Perineurioma, Hybrid nerve sheath tumor, Malignant melanotic nerve sheath tumor, Malignant peripheral nerve sheath tumor, Paraganglioma, Meningiomas, Meningioma, Mesenchymal, non-meningothelial tumors, Soft tissue tumors, Fibroblastic and myofibroblastic tumors, Solitary fibrous tumor, Vascular tumors, Hemangiomas and vascular malformations, Hemangioblastoma, Skeletal muscle tumors, Rhabdomyosarcoma, Uncertain differentiation, Intracranial mesenchymal tumor, FET-CREB fusion-positive, CIC-rearranged sarcoma, Primary intracranial sarcoma, DICER1-mutant, Ewing sarcoma, Chondro-osseous tumors, Chondrogenic tumors, Mesenchymal chondrosarcoma, Chondrosarcoma, Notochordal tumors, Chordoma (including poorly differentiated chordoma), Melanocytic tumors, Diffuse meningeal melanocytic neoplasms, Meningeal melanocytosis and meningeal melanomatosis, Circumscribed meningeal melanocytic neoplasms, Meningeal melanocytoma and meningeal melanoma, Hematolymphoid tumors, Lymphomas, CNS lymphomas, Primary diffuse large B-cell lymphoma of the CNS, Immunodeficiency-associated CNS lymphoma, Lymphomatoid granulomatosis, Intravascular large B-cell lymphoma, Miscellaneous rare lymphomas in the CNS, MALT lymphoma of the dura, Other low-grade B-cell lymphomas of the CNS, Anaplastic large cell lymphoma (ALK+/ALK−), T-cell and NK/T-cell lymphomas, Histiocytic tumors, Erdheim-Chester disease, Rosai-Dorfman disease, Juvenile xanthogranuloma, Langerhans cell histiocytosis, Histiocytic sarcoma, Germ cell tumors, Mature teratoma, Immature teratoma, Teratoma with somatic-type malignancy, Germinoma, Embryonal carcinoma, Yolk sac tumor, Choriocarcinoma, Mixed germ cell tumor, Tumors of the sellar region, Adamantinomatous craniopharyngioma, Papillary craniopharyngioma, Pituicytoma, granular cell tumor of the sellar region, and spindle cell oncocytoma, Pituitary adenoma/PitNET, Pituitary blastoma, Metastases to the CNS, Metastases to the brain and spinal cord parenchyma, and Metastases to the meninges.

124. The method, use, or compound for use of any one of claims 118 to 123 wherein the cancer is a CNS neoplasm selected from the group consisting of one or more of Pilocytic astrocytomas, Diffuse astrocytomas, Anaplastic astrocytomas, Glioblastomas, Oligodendroglial tumors, Ependymal tumor, Medulloblastomas, Pineal tumors, Meningeal tumors, and Germ cell tumors

125. The method, use, or compound for use of any one of claims 118 to 124, wherein the administration reduces one or more symptom of the cancer.

126. The method, use, or compound for use of any one of claims 118 to 125, wherein the administration reduces tumor growth.

127. The method, use, or compound for use of any one of claims 118 to 126, wherein the administration provides one or more of (i) reduction in tumor size, (ii) progression-free survival, (iii) overall survival, (iv) patient-reported outcomes, (v) disease-free survival, (vi) objective response, (vii) complete response, (viii) increased time to progression, (ix) reduced corticosteroid use, (x) reduced supportive medication, (xi) reduced incidence of seizures, (xii) reduced use of anti seizure medication, (xiii) increased qualify of life, (xiv) reduced neurological deficits, and (xv) other objective response.

128. The method, use, or compound for use of any one of claims 118 to 127, wherein the total weekly dose of ONC-206 is about 300 mg.

129. The method, use, or compound for use of any one of claims 118 to 128, wherein the total weekly AUClast is lower than about 5270 hr*ng/mL.

130. A dosing regimen comprising:
administering a dose of ONC-206: 7-benzyl-4-(2,4-difluorobenzyl)-2,4,6,7,8,9-hexahydroimidazo[1,2-a]pyrido[3,4-e]pyrimidin-5(1H)-one, or a salt thereof, twice daily for at least one day followed by a drug holiday of at least one day.

131. The dosing regimen of claim 130, wherein ONC-206 is administered twice daily for two or more consecutive days followed by a drug holiday of at least one day.

132. The dosing regimen of claim 130 or 131, wherein ONC-206 is administered twice daily for two or more consecutive days followed by a drug holiday of at least two consecutive days.

133. The dosing regimen of any one of claims 130 to 132, wherein ONC-206 is administered twice daily for three or more consecutive days followed by a drug holiday of at least two consecutive days.

134. The dosing regimen of any one of claims 130 to 133, wherein ONC-206 is administered twice daily for three or more consecutive days followed by a drug holiday of at least three consecutive days.

135. The dosing regimen of any one of claims 130 to 134, wherein ONC-206 is administered twice daily for three or more consecutive days followed by a drug holiday of at least four consecutive days.

136. The dosing regimen of any one of claims 130 to 135, wherein the dose of ONC-206 is from about 5 mg to about 150 mg.

137. The dosing regimen of any one of claims 130 to 136, wherein the dose of ONC-206 is from about 25 mg to about 100 mg.

138. The dosing regimen of any one of claims 130 to 137, wherein the dose of ONC-206 is one of 25 mg, 50 mg, 75 mg, or 100 mg.

139. The dosing regimen of any one of claims 130 to 138, wherein the dose of ONC-206 is 50 mg.

140. The dosing regimen of any one of claims 130 to 139, wherein the regimen further comprises administration of one or more additional therapeutic agent.

141. The dosing regimen of any one of claims 130 to 140, wherein the regimen further comprises ultrasound imaging.
